(19) United States
(12) Patent Application Publication
(10) Pub. No.: US 2024/0301375 A1
(43) Pub. Date: Sep. 12, 2024

(54) CIITA TARGETING ZINC FINGER NUCLEASES

(71) Applicant: Sangamo Therapeutics, Inc., Richmond, CA (US)

(72) Inventors: Lei ZHANG, Davis, CA (US); Andreas REIK, Vallejo, CA (US)

(21) Appl. No.: 18/563,741

(22) PCT Filed: May 24, 2022
(86) PCT No.: PCT/US2022/030727

§ 371 Date: Nov. 22, 2023

Related U.S. Application Data
(60) Provisional application No. 63/202,029, filed on May 24, 2021.

(51) Int. Cl. C12N 9/22

(52) U.S. Cl. CPC C12N 9/22

(57) ABSTRACT

Disclosed herein are zinc finger nucleases for cleaving a CIITA gene, polynucleotides encoding the same, and methods of using the zinc finger nucleases to modulate the expression of a CIITA gene. Also provided is a cell modified by the zinc finger nucleases and uses of the cell to treat a disease.

### CROSS REFERENCE TO RELATED APPLICATIONS

This application claims the benefit of the filing date of U.S. Provisional Patent Application No. 63/202,029, filed May 24, 2021, the contents of which are incorporated herein by reference in their entirety.

### REFERENCE TO SEQUENCE LISTING SUBMITTED ELECTRONICALLY

The content of the electronically submitted sequence listing in ASCII text file (Name: 4341_023PC01_SeqListing_ST25.txt; Size: 116,913 bytes; and Date of Creation: May 18, 2022) filed with the application is incorporated herein by reference in its entirety.

### FIELD

The present disclosure is related to zinc finger nucleases that can modulate the expression of a CIITA gene and/or protein in cells and the cells prepared by the zinc finger nucleases to treat a disease.

### BACKGROUND OF THE DISCLOSURE

Zinc-finger nucleases (ZFNs) are artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes. By taking advantage of endogenous DNA repair machinery, these reagents can be used to precisely alter the genomes of higher organisms.

ZFNs work as DNA-binding domains recognizing trinucleotide DNA sequences, with proteins linked in series to enable recognition of longer DNA sequences, thereby generating sequence recognition specificity. The fused FokI functions as a dimer, so ZFNs are engineered in pairs to recognize nucleotide sequences in close proximity. This ensures DSBs are only produced when two ZFNs simultaneously bind to opposite strands of the DNA, whereby the sequence recognition specificity is determined, inter alia, by the length of aligned DNA-binding domains. This limits off-target effects, but with the downside that arrays of zinc finger motifs influence neighboring zinc finger specificity, making their design and selection challenging. Early studies relied on delivery of the ZFN expression cassette to cells via DNA fragments derived from viral vectors. Studies later progressed to using mRNA delivery via electroporation to enable entry into target cells. This approach offers transient but high levels of the expression cassette within cells, presenting a lower risk of insertion/mutagenesis at off-target sites as a result of the shorter mRNA half-life compared to DNA.

ZFNs can be used to modulate expression of a gene in a cell. Therefore, there is a need for ZFNs that can precisely modulate gene expression in a cell for cell therapy.

### SUMMARY OF THE DISCLOSURE

In some aspects, the present disclosure provides a polynucleotide comprising a nucleic acid sequence encoding a zinc finger nuclease (ZFN) that cleaves a CIITA gene, wherein the ZFN comprises a Zinc Finger DNA-binding domain that binds to a DNA sequence in the CIITA gene and a cleavage domain, wherein the ZFN is capable of cleaving the CIITA gene between amino acids 28 and 29 corresponding to SEQ ID NO: 1 or between amino acids 461 and 462 corresponding to SEQ ID NO: 1. In some aspects, the ZEN is capable of cleaving the CIITA gene between amino acids 28 and 29 corresponding to SEQ ID NO: 1. In some aspects, the ZFN is capable of cleaving the CIITA gene between amino acids 461 and 462 corresponding to SEQ ID NO: 1.

In some aspects, the DNA-binding domain binds to GCCACCATGGAGTTG (SEQ ID NO: 9) and/or CTAGAAGGTGGCTACCTG (SEQ ID NO:15). In some aspects, wherein the DNA-binding domain binds to GCCACCATGGAGTTG (SEQ ID NO: 9). In some aspects, the DNA-binding domain binds to CTAGAAGGTGGCTACCTG (SEQ ID NO: 15). In some aspects, the DNA-binding domain binds to ATTGCT and GAACCGTCCGGG (SEQ ID NO: 38) or GATCCTGCAGGCCAT (SEQ ID NO: 29). In some aspects, the DNA-binding domain binds to ATTGCT and GAACCGTCCGGG (SEQ ID NO: 38). In some aspects, the DNA-binding domain binds to GATCCTGCAGGCCAT (SEQ ID NO: 29).

In some aspects, the DNA-binding domain comprises five zinc fingers, which comprises finger 1 (F1) comprising SEQ ID NO: 10 [RPYTLRL], finger 2 (F2) comprising SEQ ID NO: 11 [RSANLTR], finger 3 (F3) comprising SEQ ID NO: 12 [RSDALST], finger 4 (F4) comprising SEQ ID NO: 13 [DRSTRTK], and finger 5 (F5) comprising SEQ ID NO: 14 [DRSTRTK]. In some aspects, the DNA-binding domain comprises six zinc fingers, which comprises F1 comprising SEQ ID NO: 16 [RSDVLSA], F2 comprising SEQ ID NO: 17 [DRSNRIK], F3 comprising SEQ ID NO: 18 [DRSHLTR], F4 comprising SEQ ID NO: 19 [LKQHLTR], F5 comprising SEQ ID NO: 20 [QSGNLAR], and F6 comprising SEQ ID NO: 21 [QSTPRTT]. In some aspects, the polynucleotide encodes a zinc finger nuclease pair comprising a first zinc finger DNA-binding domain and a second zinc finger DNA-binding domain, wherein the first DNA-binding domain comprises five zinc fingers, which comprises finger 1 (F1) comprising SEQ ID NO: 10 [RPYTLRL], finger 2 (F2) comprising SEQ ID NO: 11 [RSANLTR], finger 3 (F3) comprising SEQ ID NO: 12 [RSDALST], finger 4 (F4) comprising 13 [DRSTRTK], and finger 5 (F5) comprising SEQ ID NO: 14 [DRSTRTK], and wherein the second DNA-binding domain comprises six zinc fingers, which comprises F1 comprising SEQ ID NO: 16 [RSDVLSA], F2 comprising SEQ ID NO: 17 [DRSNRIK], F3 comprising SEQ ID NO: 18 [DRSHLTR], F4 comprising SEQ ID NO: 19 [LKQHLTR], F5 comprising SEQ ID NO: 20 [QSGNLAR], and F6 comprising SEQ ID NO: 21 [QSTPRTT]. In some aspects, the DNA-binding domain comprises six zinc fingers, which comprises F1 comprising SEQ ID NO: 23 [RSDHLSR], F2 comprising SEQ ID NO: 24 [DSSDRKK], F3 comprising SEQ ID NO: 25 [RSDTLSE], F4 comprising 26 [QSGDLTR], and F5 comprising SEQ ID NO: 27 [QSSDLSR], and F6 comprising SEQ ID NO: 28 [YKWTLRN]. In some aspects, the DNA-binding domain comprises five zinc fingers, which comprises F1 comprising SEQ ID NO: 30 [SNQNLTT], F2 comprising SEQ ID NO: 31 [DRSHLAR], F3 comprising SEQ ID NO: 32 [QSGDLTR], F4 comprising SEQ ID NO: 33 [WKHDLTN], and F5 comprising SEQ ID NO: 34 [TSGNLTR]. In some aspects, the polynucleotide encodes a zinc finger DNA-binding domain comprising SEQ ID NO: 54. In some aspects, the polynucleotide encodes a zinc finger DNA-binding domain comprising SEQ ID NO: 56. In some aspects, the polynucleotide encodes a zinc finger nuclease pair comprising a first zinc finger DNA-binding domain and a second zinc finger DNA-binding domain, wherein the first DNA-binding domain comprises SEQ ID NO: 54 and the second DNA-binding domain comprises SEQ ID NO: 56. In some aspects, the polynucleotide encodes a zinc finger nuclease pair comprising a first zinc finger DNA-binding domain and a second zinc finger DNA-binding domain, wherein the first DNA-binding domain comprises six zinc fingers, which comprises F1 comprising SEQ ID NO: 23 [RSDHLSR], F2 comprising SEQ ID NO: 24 [DSSDRKK], F3 comprising SEQ ID NO: 25 [RSDTLSE], F4 comprising 26 [QSGDLTR], and F5 comprising SEQ ID NO: 27 [QSSDLSR], and F6 comprising SEQ ID NO: 28 [YKWTLRN], and wherein the second DNA-binding domain comprises five zinc fingers, which comprises F1 comprising SEQ ID NO: 30 [SNQNLTT], F2 comprising SEQ ID NO: 31 [DRSHLAR], F3 comprising SEQ ID NO: 32 [QSGDLTR], F4 comprising SEQ ID NO: 33 [WKHDLTN], and F5 comprising SEQ ID NO: 34 [TSGNLTR].

In some aspects, the cleavage domain comprises a FokI cleavage domain. In some aspects, the FokI cleavage domain further comprises one or more mutations at positions 418, 432, 441, 448, 476, 479, 481, 483, 486, 487, 490, 496, 499, 523, 525, 527, 537, 538 and 559 of SEQ ID NO 35. In some aspects, the one or more mutations are at positions 479, 486, 496, 499 and/or 525. In some aspects, the FokI cleavage domain comprise SEQ ID NO: 36 (FokELD). In some aspects, the one or more mutations are at positions 490, 537, and/or 538. In some aspects, the FokI cleavage domain comprise SEQ ID NO: 37 (FokKKR). In some aspects, the FokI cleavage domain forms a dimer prior to DNA cleavage. In some aspects, the FokI dimer comprises a heterodimer. In some aspects, the FokI heterodimer comprises a FokIELD dimer and a FokIKKR dimer.

In some aspects, the polynucleotide encodes a ZFN comprising the amino acid sequence having at least about 70%, at least about 80%, at least about 85%, at least about 90%, at least about 95%, at least about 96%, at least about 97%, at least about 98%, at least about 99%, or about 100% sequence identity to SEQ ID NO: 5. In some aspects, the polynucleotide encodes a ZFN comprising the amino acid sequence having at least about 70%, at least about 80%, at least about 85%, at least about 90%, at least about 95%, at least about 96%, at least about 97%, at least about 98%, at least about 99%, or about 100% sequence identity to SEQ ID NO: 6.

In some aspects, the present disclosure provides a polynucleotide comprising a sequence at least about 70%, at least about 80%, at least about 90%, at least about 95%, at least about 96%, at least about 97%, or least about 98%, at least about 99% or about 100% sequence identity to SEQ ID NO 39.

In some aspects, the present disclosure provides polynucleotides encoding a ZFN wherein the ZFN comprises the amino acid sequence having at least about 70%, at least about 80%, at least about 85%, at least about 90%, at least about 95%, at least about 96%, at least about 97%, at least about 98%, at least about 99%, or about 100% sequence identity to SEQ ID NO: 7. In some aspects, the present disclosure provides polynucleotides encoding a ZFN wherein the ZFN comprises the amino acid sequence having at least about 70%, at least about 80%, at least about 85%, at least about 90%, at least about 95%, at least about 96%, at least about 97%, at least about 98%, at least about 99%, or about 100% sequence identity to SEQ ID NO: 8.

In some aspects, the present disclosure provides a polynucleotide comprising a sequence at least about 70%, at least about 80%, at least about 90%, at least about 95%, at least about 96%, at least about 97%, or least about 98%, at least about 99% sequence identity to SEQ ID NO: 40, SEQ ID NO: 53, SEQ ID NO: 55, or SEQ ID NO: 57.

In some aspects, the present disclosure provides a polynucleotide comprising six zinc fingers, which comprises F1 comprising SEQ ID NO: 23 [RSDHLSR], F2 comprising SEQ ID NO: 24 [DSSDRKK], F3 comprising SEQ ID NO: 25 [RSDTLSE], F4 comprising 26 [QSGDLTR], and F5 comprising SEQ ID NO: 27 [QSSDLSR], and F6 comprising SEQ ID NO: 28 [YKWTLRN] and a FokI cleavage domain, wherein the FokI cleavage domain further comprises K to S mutation at position 525 of SEQ ID NO 35.

In some aspects, the present disclosure provides a polynucleotide comprising five zinc fingers, which comprises F1 comprising SEQ ID NO: 30 [SNQNLTT], F2 comprising SEQ ID NO: 31 [DRSHLAR], F3 comprising SEQ ID NO: 32 [QSGDLTR], F4 comprising SEQ ID NO: 33 [WKHDLTN], and F5 comprising SEQ ID NO: 34 [TSGNLTR] and a FokI cleavage domain, wherein the FokI cleavage domain further comprises I to T mutation at position 479 of SEQ ID NO 35.

In some aspects, the present disclosure provides a zinc finger nuclease (ZFN) that cleaves a CIITA gene, wherein the ZFN comprises a Zinc Finger DNA-binding domain that binds to a DNA sequence in the CIITA gene and a cleavage domain, wherein the ZFN is capable of cleaving the CIITA gene between amino acids 28 and 29 corresponding to SEQ ID NO: 1 or between amino acids 461 and 462 corresponding to SEQ ID NO: 1. In some aspects, the ZEN is capable of cleaving the CIITA gene between amino acids 28 and 29 corresponding to SEQ ID NO: 1. In some aspects, the ZFN is capable of cleaving the CIITA gene between amino acids 461 and 462 corresponding to SEQ ID NO: 1. In some aspects, the ZFP DNA-binding domain binds to GCCACCATGGAGTTG (SEQ ID NO: 9) and/or CTAGAAGGTGGCTACCTG (SEQ ID NO: 15). In some aspects, the ZFP DNA-binding domain binds to GCCACCATGGAGTTG (SEQ ID NO: 9). In some aspects, the ZFP DNA-binding domain binds to CTAGAAGGTGGCTACCTG (SEQ ID NO: 15). In some aspects, the ZFP DNA-binding domain binds to ATTGCT and GAACCGTCCGGG (SEQ ID NO: 38) or GATCCTGCAGGCCAT (SEQ ID NO: 29). In some aspects, the ZFP DNA-binding domain binds to ATTGCT and GAACCGTCCGGG (SEQ ID NO: 38). In some aspects, the ZFP DNA-binding domain binds to GATCCTGCAGGCCAT (SEQ ID NO: 29).

In some aspects, the ZFP DNA-binding domain comprises five zinc fingers, which comprises finger 1 (F1) comprising SEQ ID NO: 10 [RPYTLRL], finger 2 (F2) comprising SEQ ID NO: 11 [RSANLTR], finger 3 (F3) comprising SEQ ID NO: 12 [RSDALST], finger 4 (F4) comprising SEQ ID NO: 13 [DRSTRTK], and finger 5 (F5) comprising SEQ ID NO: 14 [DRSTRTK]. In some aspects, the ZFP DNA-binding domain comprises six zinc fingers, which comprises F1 comprising SEQ ID NO: 16 [RSDVLSA], F2 comprising SEQ ID NO: 17 [DRSNRIK], F3 comprising SEQ ID NO: 18 [DRSHLTR], F4 comprising SEQ ID NO: 19 [LKQHLTR], F5 comprising SEQ ID NO: 20 [QSGNLAR], and F6 comprising SEQ ID NO: 21 [QSTPRTT].

In some aspects, the present disclosure provides a ZFN which comprises a ZFN pair comprising a first zinc finger DNA-binding domain and a second zinc finger DNA-binding domain, wherein the first DNA-binding domain comprises five zinc fingers, which comprises finger 1 (F1) comprising SEQ ID NO: 10 [RPYTLRL], finger 2 (F2) comprising SEQ ID NO: 11 [RSANLTR], finger 3 (F3) comprising SEQ ID NO: 12 [RSDALST], finger 4 (F4) comprising 13 [DRSTRTK], and finger 5 (F5) comprising SEQ ID NO: 14 [DRSTRTK], and wherein the second DNA-binding domain comprises six zinc fingers, which comprises F1 comprising SEQ ID NO: 16 [RSDVLSA], F2 comprising SEQ ID NO: 17 [DRSNRIK], F3 comprising SEQ ID NO: 18 [DRSHLTR], F4 comprising SEQ ID NO: 19 [LKQHLTR], F5 comprising SEQ ID NO: 20 [QSGNLAR], and F6 comprising SEQ ID NO: 21 [QSTPRTT]. In some aspects, the DNA-binding domain comprises six zinc fingers, which comprises F1 comprising SEQ ID NO: 23 [RSDHLSR], F2 comprising SEQ ID NO: 24 [DSSDRKK], F3 comprising SEQ ID NO: 25 [RSDTLSE], F4 comprising 26 [QSGDLTR], and F5 comprising SEQ ID NO: 27 [QSSDLSR], and F6 comprising SEQ ID NO: 28 [YKWTLRN]. In some aspects, the DNA-binding domain comprises five zinc fingers, which comprises F1 comprising SEQ ID NO: 30 [SNQNLTT], F2 comprising SEQ ID NO: 31 [DRSHLAR], F3 comprising SEQ ID NO: 32 [QSGDLTR], F4 comprising SEQ ID NO: 33 [WKHDLTN], and F5 comprising SEQ ID NO: 34 [TSGNLTR]. In some aspects, a ZFN pair comprises a first zinc finger DNA-binding domain and a second zinc finger DNA-binding domain, wherein the first DNA-binding domain comprises six zinc fingers, which comprises F1 comprising SEQ ID NO: 23 [RSDHLSR], F2 comprising SEQ ID NO: 24 [DSSDRKK], F3 comprising SEQ ID NO: 25 [RSDTLSE], F4 comprising SEQ ID NO: 26 [QSGDLTR], and F5 comprising SEQ ID NO: 27 [QSSDLSR], and F6 comprising SEQ ID NO: 28 [YKWTLRN], and wherein the second DNA-binding domain comprises five zinc fingers, which comprises F1 comprising SEQ ID NO: 30 [SNQNLTT], F2 comprising SEQ ID NO: 31 [DRSHLAR], F3 comprising SEQ ID NO: 32 [QSGDLTR], F4 comprising SEQ ID NO: 33 [WKHDLTN], and F5 comprising SEQ ID NO: 34 [TSGNLTR]. In some aspects, the DNA-binding domain comprises SEQ ID NO: 54. In some aspects, the DNA-binding domain comprises SEQ ID NO: 56. In some aspects, the ZFN comprises a ZFN pair comprising a first zinc finger DNA-binding domain and a second zinc finger DNA-binding domain, wherein the first DNA-binding domain comprises SEQ ID NO: 54 and the second DNA-binding domain comprises SEQ ID NO: 56.

In some aspects, the ZFN comprises the cleavage domain comprising a FokI cleavage domain. In some aspects, the FokI cleavage domain further comprises one of more mutations at positions 418, 432, 441, 448, 476, 481, 483, 486, 487, 490, 496, 499, 523, 527, 537, 538 and 559 of SEQ ID NO 35. In some aspects, the one or more mutations are at positions 486, 496, and/or 499. In some aspects, wherein the FokI cleavage domain comprises SEQ ID NO: 36 (FokELD). In some aspects, the one or more mutations are at positions 490, 537, and/or 538. In some aspects, the FokI cleavage domain comprises SEQ ID NO: 37 (FokKKR). In some aspects, the FokI cleavage domain forms a dimer prior to DNA cleavage. In some aspects, the FokI dimer comprises a heterodimer. In some aspects, the FokI heterodimer comprises a FokELD dimer and a FokKKR dimer.

In some aspects, the ZFN comprises the amino acid sequence having at least about 70%, at least about 80%, at least about 85%, at least about 90%, at least about 95%, at least about 96%, at least about 97%, at least about 98%, at least about 99%, or about 100% sequence identity to SEQ ID NO: 5. In some aspects, the ZFN comprises the amino acid sequence having at least about 70%, at least about 80%, at least about 85%, at least about 90%, at least about 95%, at least about 96%, at least about 97%, at least about 98%, at least about 99%, or about 100% sequence identity to SEQ ID NO: 6. In some aspects, the ZFN comprises the amino acid sequence having at least about 70%, at least about 80%, at least about 85%, at least about 90%, at least about 95%, at least about 96%, at least about 97%, at least about 98%, at least about 99%, or about 100% sequence identity to SEQ ID NO: 7. In some aspects, the ZFN comprises the amino acid sequence having at least about 70%, at least about 80%, at least about 85%, at least about 90%, at least about 95%, at least about 96%, at least about 97%, at least about 98%, at least about 99%, or about 100% sequence identity to SEQ ID NO: 8.

In some aspects, the ZFN comprises the DNA-binding domain comprising six zinc fingers, which comprises F1 comprising SEQ ID NO: 23 [RSDHLSR], F2 comprising SEQ ID NO: 24 [DSSDRKK], F3 comprising SEQ ID NO: 25 [RSDTLSE], F4 comprising 26 [QSGDLTR], and F5 comprising SEQ ID NO: 27 [QSSDLSR], and F6 comprising SEQ ID NO: 28 [YKWTLRN] and a FokI cleavage domain, wherein the FokI cleavage domain further comprises K to S mutation at position 525 of SEQ ID NO 35.

In some aspects, the ZFN comprises the DNA-binding domain comprising five zinc fingers, which comprises F1 comprising SEQ ID NO: 30 [SNQNLTT], F2 comprising SEQ ID NO: 31 [DRSHLAR], F3 comprising SEQ ID NO: 32 [QSGDLTR], F4 comprising SEQ ID NO: 33 [WKHDLTN], and F5 comprising SEQ ID NO: 34 [TSGNLTR] and a FokI cleavage domain, wherein the FokI cleavage domain further comprises I to T mutation at position 479 of SEQ ID NO 35.

In some aspects, the present disclosure provides a ZFN pair comprising a first ZFN and a second ZFN, wherein the first ZFN comprises the amino acid sequence having at least about 70%, at least about 80%, at least about 85%, at least about 90%, at least about 95%, at least about 96%, at least about 97%, at least about 98%, at least about 99%, or about 100% sequence identity to SEQ ID NO: 5, and the second ZFN comprises the amino acid sequence having at least about 70%, at least about 80%, at least about 85%, at least about 90%, at least about 95%, at least about 96%, at least about 97%, at least about 98%, at least about 99%, or about 100% sequence identity to SEQ ID NO: 6.

In some aspects, the present disclosure provides a ZFN pair comprising a first ZFN and a second ZFN, wherein the first ZFN comprises the amino acid sequence having at least about 70%, at least about 80%, at least about 85%, at least about 90%, at least about 95%, at least about 96%, at least about 97%, at least about 98%, at least about 99%, or about 100% sequence identity to SEQ ID NO: 7, and the second ZFN comprises the amino acid sequence having at least about 70%, at least about 80%, at least about 85%, at least about 90%, at least about 95%, at least about 96%, at least about 97%, at least about 98%, at least about 99%, or about 100% sequence identity to SEQ ID NO: 8. In some aspects, the ZFN pair comprises a first ZFN and a second ZFN, wherein the first ZFN comprises the amino acid sequence having at least about 70%, at least about 80%, at least about 85%, at least about 90%, at least about 95%, at least about 96%, at least about 97%, at least about 98%, at least about 99%, or about 100% sequence identity to SEQ ID NO: 5444, and the second ZFN comprises the amino acid sequence having at least about 70%, at least about 80%, at least about 85%, at least about 90%, at least about 95%, at least about 96%, at least about 97%, at least about 98%, at least about 99%, or about 100% sequence identity to SEQ ID NO: 56.

In some aspects, the present disclosure provides a ZFN pair comprising a first zinc finger DNA-binding domain, a first cleavage domain, a second zinc finger DNA-binding domain, and a second cleavage domain, wherein the first DNA-binding domain comprises six zinc fingers, which comprises F1 comprising SEQ ID NO: 23 [RSDHLSR], F2 comprising SEQ ID NO: 24 [DSSDRKK], F3 comprising SEQ ID NO: 25 [RSDTLSE], F4 comprising SEQ ID NO: 26 [QSGDLTR], and F5 comprising SEQ ID NO: 27 [QSSDLSR], and F6 comprising SEQ ID NO: 28 [YKWTLRN], and wherein the second DNA-binding domain comprises five zinc fingers, which comprises F1 comprising SEQ ID NO: 30 [SNQNLTT], F2 comprising SEQ ID NO: 31 [DRSHLAR], F3 comprising SEQ ID NO: 32 [QSGDLTR], F4 comprising SEQ ID NO: 33 [WKHDLTN], and F5 comprising SEQ ID NO: 34 [TSGNLTR], wherein the first and the second cleavage domain comprise a FokI cleavage domain, and wherein the first FokI cleavage domain further comprises K to S mutation at position 525 of SEQ ID NO 35 and the second FokI cleavage domain further comprises I to T mutation at position 479 of SEQ ID NO 35.

In some aspects, the present disclosure provides a ZFN encoded by the polynucleotides disclosed herein. In some aspects, the present disclosure provides a polynucleotides encoding the ZFNs disclosed herein or the ZFN pairs disclosed herein.

In some aspects, the present disclosure provides an isolated cell comprising the polynucleotides, the ZFNs, or the ZFN pairs disclosed herein.

In some aspects, the isolated cell comprises a T cell, a NK cell, a tumor infiltrating lymphocyte, a stem cell, Mesenchymal stem cells (MSC), hematopoietic stem cells (HSC), fibroblasts, cardiomyocytes, pancreatic islet cells, or a blood cell. In some aspects, the cell is allogeneic. In some aspects, the cell is autologous.

In some aspects, the present disclosure provides a method of preparing a T cell, comprising contacting an isolated T cell with the polynucleotides, the ZFNs, or the ZFN pairs disclosed herein. In some aspects, the T cell comprises a chimeric antigen receptor T cell, a T cell receptor cell, a Treg cell, a Tumor infiltrating lymphocyte, or any combination thereof. In some aspects, the present disclosure provides a method of treating a subject in need of a cellular therapy comprising administering the isolated cell disclosed herein. In some aspects, the administered isolated cells are allogeneic or autologous.

### BRIEF DESCRIPTION OF THE DRAWINGS

FIG. 1 shows CIITA gene location, transcript, protein sequence and ZFN cutting sites.

FIG. 2A shows full-length protein sequence alignments of CIITA from 9 species. FIGS. 2B and 2C show the cutting sites in the CIITA gene for ZFN pairs 76867:82862 (site B) and 87254:84221 (site G), respectively.

FIG. 3 shows a schematic diagram of design information, architectures, and DNA binding sequences for ZFN pairs 76867:82862 (site B) and 87254:84221 (site G). The respective DNA binding sites are shown in bold.

FIG. 4 shows fluorescence-activated cell sorting (FACS) analysis of MHC class II levels in CIITA ZFN transfected T cells. Top panels show ZFN 76867-2A-82862. Bottom panels show ZFN 87254-2A-84221. A concentration dependent reduction in MHC class II signal in the CIITA ZFN treated samples compared to the mock and TRAC ZFN control samples was observed.

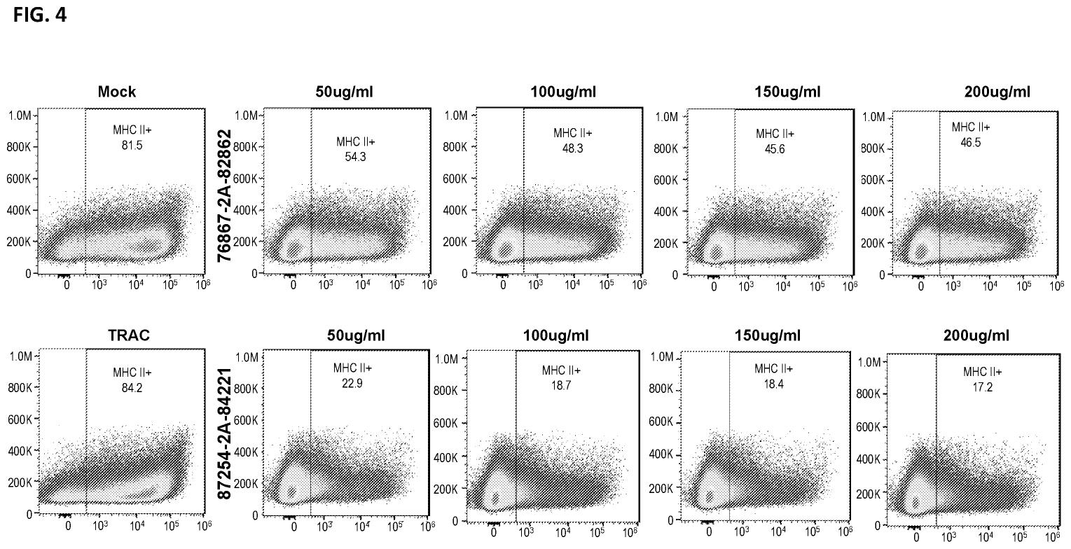

FIG. 5A shows a schematic diagram of a polynucleotide construct for generation of mRNA from ZFN pairs 87278-2A-87232. FIG. 5B shows the DNA binding sites in Exon 11 of human CIITA gene for ZFN pairs 87278-2A-87232 (site G). The respective DNA binding sites are shown in bold.

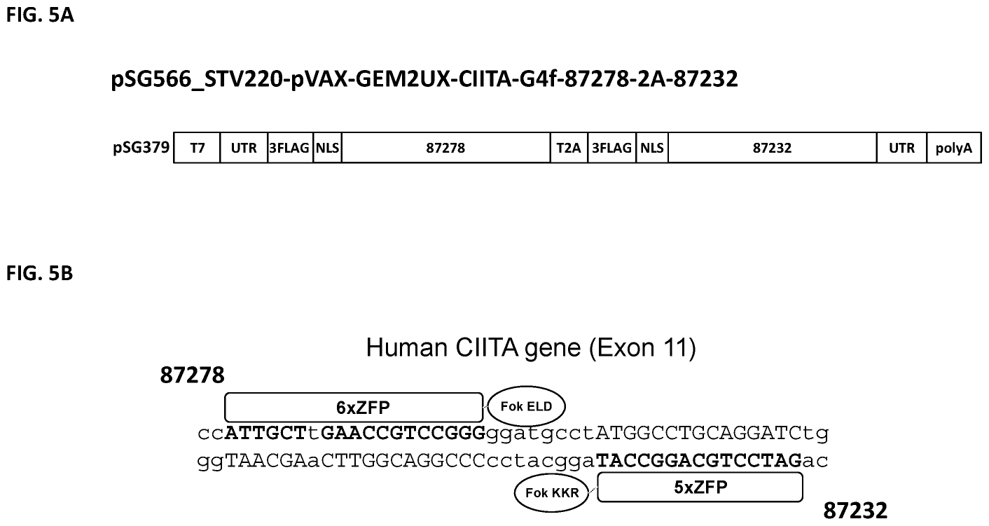

FIG. 6 shows an experimental plan for CD4+/CD127Low/CD25+ Treg cells activation and immuno-phenotyping.

FIGS. 7A and 7B show ZFN-mediated CIITA gene editing and MHCII knock-out in Treg cells. mRNA encoding CIITA targeting ZFNs (87278 and 87232) was electroporated at different concentrations (0, 30, 60, 90, 120 μg/mL) in isolated Treg cells. Editing efficiency was quantified by determining the percentage of indels (% of indels) (FIG. 7A) as well as measuring the percentage of cells expressing MHCII at the cell surface (% of MHCII⁺ cells) (FIG. 7B).

### DETAILED DESCRIPTION OF THE DISCLOSURE

### I. Overview

The present disclosure is directed to a polynucleotide (e.g., isolated polynucleotide) comprising a nucleotide sequence encoding a zinc finger nuclease (ZFN) that cleaves a CIITA gene, wherein the ZFN comprises a zinc finger DNA-binding domain that binds to a DNA sequence in the CIITA gene, and a cleavage domain.

### II. Definitions

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

As used herein, the term “approximately” or “about,” as applied to one or more values of interest, refers to a value that is similar to a stated reference value and within a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). When the term “approximately” or “about” is applied herein to a particular value, the value without the term “approximately” or “about is also disclosed herein.

As described herein, any concentration range, percentage range, ratio range, or integer range is to be understood to include the value of any integer within the recited range and, when appropriate, fractions thereof (such as one tenth and one hundredth of an integer), unless otherwise indicated.

As used herein, the terms “ug” and “uM” are used interchangeably with “μg” and “μM,” respectively.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, the term “immune cell” refers to a cell of the immune system. In some aspects, the immune cell is selected from a T lymphocyte (“T cell”), B lymphocyte (“B cell”), natural killer (NK) cell, natural killer T (NKT) cell, macrophage, eosinophil, mast cell, dendritic cell or neutrophil). As used herein, the terms “T cell” and “T lymphocyte” are interchangeable and refer to any lymphocytes produced or processed by the thymus gland. Non-limiting classes of T cells include effector T cells and Th cells (such as CD4⁺ or CD8⁺ T cells). In some aspects, the immune cell is a Th1 cell. In some aspects, the immune cell is a Th2 cell. In some aspects, the immune cell is a Tc17 cell. In some aspects, the immune cell is a Th17 cell. In some aspects, the immune cell is a tumor-infiltrating cell (TIL). In some aspects, the immune cell is a Treg cell. As used herein, an “immune cell” also refers to a pluripotent cell, e.g., a stem cell (e.g., an embryonic stem cell or a hematopoetic stem cell) or an induced pluripotent stem cell, or a progenitor cell which is capable of differentiation into an immune cell.

In some aspects, the T cell is a memory T cell. As used herein, the term “memory” T cells refers to T cells that have previously encountered and responded to their cognate antigen (e.g., in vivo, in vitro, or ex vivo) or which have been stimulated with, e.g., an anti-CD3 antibody (e.g., in vitro or ex vivo). Immune cells having a “memory-like” phenotype upon secondary exposure, such memory T cells can reproduce to mount a faster and strong immune response than during the primary exposure. In some aspects, memory T cells comprise central memory T cells (T_{CM} cells), effector memory T cells (T_{EM} cells), tissue resident memory T cells (T_{RM} cells), stem cell-like memory T cells (T_{SCM} cells), or any combination thereof.

In some aspects, the T cell is a stem cell-like memory T cell. As used herein, the term “stem cell-like memory T cells,” “T memory stem cells,” or “T_{SCM} cells” refer to memory T cells that express CD95, CD45RA, CCR7, and CD62L and are endowed with the stem cell-like ability to self-renew and the multipotent capacity to reconstitute the entire spectrum of memory and effector subsets.

In some aspects, the T cell is a central memory T cell. As used herein, the term “central memory T cells” or “T_{CM} cells” refer to memory T cells that express CD45RO, CCR7, and CD62L. Central memory T cells are generally found within the lymph nodes and in peripheral circulation.

In some aspects, the T cell is an effector memory T cell. As used herein, the term “effector memory T cells” or “T_{EM} cells” refer to memory T cells that express CD45RO but lack expression of CCR7 and CD62L. Because effector memory T cells lack lymph node-homing receptors (e.g., CCR7 and CD62L), these cells are typically found in peripheral circulation and in non-lymphoid tissues.

In some aspects, the T cell is a tissue resident memory T cell. As used herein, the term “tissue resident memory T cells” or “T_{RM} cells” refer to memory T cells that do not circulate and remain resident in peripheral tissues, such as the skin, lung, and the gastrointestinal tract. In certain aspects, tissue resident memory T cells are also effector memory T cells.

In some aspects, the T cell is a naïve T cell. As used herein, the term “naïve T cells” or “T_{N} cells” refers to T cells that express CD45RA, CCR7, and CD62L, but which do not express CD95. T_{N} cells represent the most undifferentiated cell in the T cell lineage. The interaction between a T_{N} cell and an antigen presenting cell (APC) induces differentiation of the T_{N} cell towards an activated T_{EFF} cell and an immune response. In some aspects, the T cell is an effector T (T_{eff}) cell.

As used herein, the term “immune response” refers to a biological response within a vertebrate against foreign agents, which response protects the organism against these agents and diseases caused by them. An immune response is mediated by the action of a cell of the immune system (e.g., a T lymphocyte, B lymphocyte, natural killer (NK) cell, NKT cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell or a Th cell, such as a CD4⁺ or CD8⁺ T cell, or the inhibition of a Treg cell. As used herein, the terms “T cell” and “T lymphocytes” are interchangeable and refer to any lymphocytes produced or processed by the thymus gland. In some aspects, a T cell is a CD4+ T cell. In some aspects, a T cell is a CD8+ T cell. In some aspects, a T cell is a NKT cell.

The terms “nucleic acids,” “nucleic acid molecules, “nucleotides,” “nucleotide(s) sequence,” and “polynucleotide” can be used interchangeably and refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Single stranded nucleic acid sequences refer to single-stranded DNA (ssDNA) or single-stranded RNA (ssRNA). Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences can be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation. DNA includes, but is not limited to, cDNA, genomic DNA, plasmid DNA, synthetic DNA, and semi-synthetic DNA. A “nucleic acid composition” of the disclosure comprises one or more nucleic acids as described herein. As described herein, in some aspects, a polynucleotide of the present disclosure can comprise a single nucleotide sequence encoding a single protein (e.g., ZFN) (“monocistronic”). In some aspects, a polynucleotide of the present disclosure is polycistronic (i.e., comprises two or more cistrons). In certain aspects, each of the cistrons of a polycistronic polynucleotide can encode for a protein disclosed herein (e.g., ZFN). In some aspects, each of the cistrons can be translated independently of one another.

In some aspects, a polynucleotide of the present disclosure is polycistronic (i.e., comprises two or more cistrons). In certain aspects, each of the cistrons of a polycistronic polynucleotide can encode for a protein disclosed herein (e.g., a first ZFN and a second ZFN). In some aspects, each of the cistrons can be translated independently of one another.

As used herein, a “coding region,” “coding sequence,” or “translatable sequence” is a portion of polynucleotide which consists of codons translatable into amino acids. Although a “stop codon” (TAG, TGA, or TAA) is typically not translated into an amino acid, it can be considered to be part of a coding region, but any flanking sequences, for example promoters, ribosome binding sites, transcriptional terminators, introns, and the like, are not part of a coding region. The boundaries of a coding region are typically determined by a start codon at the 5′ terminus, encoding the amino terminus of the resultant polypeptide, and a translation stop codon at the 3′ terminus, encoding the carboxyl terminus of the resulting polypeptide.

The terms “polypeptide,” “peptide” and “protein” are used interchangeably to refer to a polymer of amino acid residues. The term also applies to amino acid polymers in which one or more amino acids are chemical analogues or modified derivatives of a corresponding naturally-occurring amino acids.

The term “expression” as used herein refers to a process by which a polynucleotide produces a gene product, for example, ZFN. It includes, without limitation, transcription of the polynucleotide into messenger RNA (mRNA) and the translation of an mRNA into a polypeptide. Expression produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation or splicing, or polypeptides with post translational modifications, e.g., methylation, glycosylation, the addition of lipids, association with other protein subunits, or proteolytic cleavage.

As used herein, the term “identity” refers to the overall monomer conservation between polymeric molecules, e.g., between polynucleotide molecules. The term “identical” without any additional qualifiers, e.g., polynucleotide A is identical to polynucleotide B, implies the polynucleotide sequences are 100% identical (100% sequence identity). Describing two sequences as, e.g., “70% identical,” is equivalent to describing them as having, e.g., “70% sequence identity.”

Calculation of the percent identity of two polypeptide or polynucleotide sequences, for example, can be performed by aligning the two sequences for optimal comparison purposes (e.g., gaps can be introduced in one or both of a first and a second polypeptide or polynucleotide sequences for optimal alignment and non-identical sequences can be disregarded for comparison purposes). In certain aspects, the length of a sequence aligned for comparison purposes is at least about 30%, at least about 40%, at least about 50%, at least about 60%, at least about 70%, at least about 80%, at least about 90%, at least about 95%, or about 100% of the length of the reference sequence. The amino acids at corresponding amino acid positions, or bases in the case of polynucleotides, are then compared.

When a position in the first sequence is occupied by the same amino acid or nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm.

Suitable software programs that can be used to align different sequences (e.g., polynucleotide sequences) are available from various sources. One suitable program to determine percent sequence identity is bl2seq, part of the BLAST suite of program available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at worldwideweb.ebi.ac.uk/Tools/psa.

Sequence alignments can be conducted using methods known in the art such as MAFFT, Clustal (ClustalW, Clustal X or Clustal Omega), MUSCLE, etc.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In certain aspects, the percentage identity (% ID) or of a first amino acid sequence (or nucleic acid sequence) to a second amino acid sequence (or nucleic acid sequence) is calculated as % ID=100×(Y/Z), where Y is the number of amino acid residues (or nucleobases) scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at worldwidewebtcoffee.org, and alternatively available, e.g., from the EBI. It will also be appreciated that the final alignment used to calculate percent sequence identity can be curated either automatically or manually.

The term “linked” as used herein refers to a first amino acid sequence or polynucleotide sequence covalently or non-covalently joined to a second amino acid sequence or polynucleotide sequence, respectively. The first amino acid or polynucleotide sequence can be directly joined or juxtaposed to the second amino acid or polynucleotide sequence or alternatively an intervening sequence can covalently join the first sequence to the second sequence. The term “linked” means not only a fusion of a first polynucleotide sequence to a second polynucleotide sequence at the 5′-end or the 3′-end, but also includes insertion of the whole first polynucleotide sequence (or the second polynucleotide sequence) into any two nucleotides in the second polynucleotide sequence (or the first polynucleotide sequence, respectively). The first polynucleotide sequence can be linked to a second polynucleotide sequence by a phosphodiester bond or a linker. The linker can be, e.g., a polynucleotide.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific. Such interactions are generally characterized by a dissociation constant (K_{d}) of 10⁻⁶ M⁻¹ or lower. “Affinity” refers to the strength of binding: increased binding affinity being correlated with a lower K_{d}. “Non-specific binding” refers to, non-covalent interactions that occur between any molecule of interest (e.g. an engineered nuclease) and a macromolecule (e.g. DNA) that are not dependent on-target sequence.

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

A “DNA binding molecule” is a molecule that can bind to DNA. Such DNA binding molecule can be a polypeptide, a domain of a protein, a domain within a larger protein or a polynucleotide. In some aspects, the polynucleotide is DNA, while in other aspects, the polynucleotide is RNA. In some aspects, the DNA binding molecule is a protein domain of a nuclease (e.g. the FokI domain). In some aspects, the DNA binding molecule binds to all nucleotides of a given sequence. In some aspects, the DNA binding molecule binds all but one nucleotides of a given sequence. In some aspects, the DNA binding molecule binds all but two or more nucleotides of a given sequence.

A “DNA binding protein” (or binding domain) is a protein, or a domain within a larger protein, that binds DNA in a sequence-specific manner, for example through one or more zinc fingers or through interaction with one or more RVDs in a zinc finger protein, respectively. The term zinc finger DNA binding protein is often abbreviated as “zinc finger protein” or “ZFP”.

A “zinc finger DNA binding protein” or “zinc finger DNA binding domain” is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as “zinc finger protein” or “ZFP”. The term “zinc finger nuclease” includes one ZFN as well as a pair of ZFNs (the members of the pair are referred to as “left and right” or “first and second” or “pair”) that dimerize to cleave the target gene. In some aspects, the zinc finger DNA binding domain binds to all nucleotides of a given sequence. In some aspects, the zinc finger DNA binding domain can bind all but one nucleotides of a given sequence. In some aspects, the zinc finger DNA binding domain can bind all but two or more nucleotides of a given sequence.

Zinc finger DNA-binding domains can be “engineered” to bind to a predetermined nucleotide sequence, for example via engineering (altering one or more amino acids) of the recognition helix region of a naturally occurring zinc finger protein or by engineering of the amino acids involved in DNA binding (the “repeat variable diresidue” or RVD region). Therefore, engineered zinc finger proteins are proteins that are non-naturally occurring. Non-limiting examples of methods for engineering zinc finger proteins are design and selection. A designed protein is a protein not occurring in nature whose design/composition results principally from rational criteria. Rational criteria for design include application of substitution rules and computerized algorithms for processing information in a database storing information of existing ZFP designs and binding data. See, for example, U.S. Pat. Nos. 8,586,526; 6,140,081; 6,453,242; and 6,534,261; see also WO 98/53058; WO 98/53059; WO 98/53060; WO 02/016536 and WO 03/016496.

A “selected” zinc finger protein is not found in nature, and whose production results primarily from an empirical process such as phage display, interaction trap, rational design or hybrid selection. See e.g., U.S. Pat. Nos. 5,789,538; 5,925,523; 6,007,988; 6,013,453; 6,200,759; WO 95/19431; WO 96/06166; WO 98/53057; WO 98/54311; WO 00/27878; WO 01/60970; WO 01/88197 and WO 02/099084.

“Recombination” refers to a process of exchange of genetic information between two polynucleotides, including but not limited to, capture by non-homologous end joining (NHEJ) and homologous recombination. For the purposes of this disclosure, “homologous recombination (HR)” refers to the specialized form of such exchange that takes place, for example, during repair of double-strand breaks in cells via homology-directed repair mechanisms. This process requires nucleotide sequence homology, uses a “donor” molecule to template repair of a “target” molecule (i.e., the one that experienced the double-strand break), and is variously known as “non-crossover gene conversion” or “short tract gene conversion,” because it leads to the transfer of genetic information from the donor to the target. Without wishing to be bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized HR often results in an alteration of the sequence of the target molecule such that part or all of the sequence of the donor polynucleotide is incorporated into the target polynucleotide.

In certain aspects of the disclosure, one or more targeted nucleases as described herein create a double-stranded break (DSB) in the target sequence (e.g., cellular chromatin) at a predetermined site (e.g., a gene or locus of interest). The DSB mediates integration of a construct (e.g. donor) as described herein or knock down of functional gene expression. Optionally, the construct has homology to the nucleotide sequence in the region of the break. An expression construct may be physically integrated or, alternatively, the expression cassette is used as a template for repair of the break via homologous recombination, resulting in the introduction of all or part of the nucleotide sequence as in the expression cassette into the cellular chromatin. Thus, a first sequence in cellular chromatin can be altered and, in certain embodiments, can be converted into a sequence present in an expression cassette. Thus, the use of the terms “replace” or “replacement” can be understood to represent replacement of one nucleotide sequence by another, (i.e., replacement of a sequence in the informational sense), and does not necessarily require physical or chemical replacement of one polynucleotide by another.

In any of the methods and compositions (e.g., nucleases, cells made using these nucleases, etc.) described herein, additional engineered nucleases can be used for additional double-stranded cleavage of additional target sites within the cell.

In some aspects of methods for targeted recombination and/or replacement and/or alteration of a sequence in a region of interest in cellular chromatin, a chromosomal sequence is altered by homologous recombination with an exogenous “donor” nucleotide sequence. Such homologous recombination is stimulated by the presence of a double-stranded break in cellular chromatin, if sequences homologous to the region of the break are present.

In any of the methods and compositions (e.g., nucleases, cells made using these nucleases, etc.) described herein, the first nucleotide sequence (the “donor sequence”) can contain sequences that are homologous, but not identical, to genomic sequences in the region of interest, thereby stimulating homologous recombination to insert a non-identical sequence in the region of interest. Thus, in certain embodiments, portions of the donor sequence that are homologous to sequences in the region of interest exhibit between about 80 to 99% (or any integer therebetween) sequence identity to the genomic sequence that is replaced. In other embodiments, the homology between the donor and genomic sequence is higher than 99%, for example if only 1 nucleotide differs as between donor and genomic sequences of over 100 contiguous base pairs. In certain cases, a non-homologous portion of the donor sequence can contain sequences not present in the region of interest, such that new sequences are introduced into the region of interest. In these instances, the non-homologous sequence is generally flanked by sequences of 50-1,000 base pairs (or any integral value therebetween) or any number of base pairs greater than 1,000, that are homologous or identical to sequences in the region of interest. In other embodiments, the donor sequence is non-homologous to the first sequence, and is inserted into the genome by non-homologous recombination mechanisms.

Any of the methods described herein can be used for partial or complete inactivation of one or more target sequences in a cell by targeted integration of donor sequence or via cleavage of the target sequence(s) followed by error-prone NHEJ-mediated repair that disrupts expression of the gene(s) of interest. Cell lines with partially or completely inactivated genes are also provided.

Furthermore, the methods of targeted integration as described herein can also be used to integrate one or more exogenous sequences. The exogenous nucleic acid sequence can comprise, for example, one or more genes or cDNA molecules, or any type of coding or noncoding sequence, as well as one or more control elements (e.g., promoters). In addition, the exogenous nucleic acid sequence may produce one or more RNA molecules (e.g., small hairpin RNAs (shRNAs), inhibitory RNAs (RNAis), microRNAs (miRNAs), etc.).

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

The term “sequence” refers to a nucleotide sequence of any length, which can be DNA or RNA; can be linear, circular or branched and can be either single-stranded or double stranded. The term “transgene” refers to a nucleotide sequence that is inserted into a genome. A transgene can be of any length, for example between 2 and 100,000,000 nucleotides in length (or any integer value therebetween or thereabove), preferably between about 100 and 100,000 nucleotides in length (or any integer therebetween), more preferably between about 2000 and 20,000 nucleotides in length (or any value therebetween) and even more preferable, between about 5 and 15 kb (or any value therebetween).

A “chromosome,” is a chromatin complex comprising all or a portion of the genome of a cell. The genome of a cell is often characterized by its karyotype, which is the collection of all the chromosomes that comprise the genome of the cell. The genome of a cell can comprise one or more chromosomes.

An “episome” is a replicating nucleic acid, nucleoprotein complex or other structure comprising a nucleic acid that is not part of the chromosomal karyotype of a cell. Examples of episomes include plasmids, minicircles and certain viral genomes. The liver specific constructs described herein may be episomally maintained or, alternatively, may be stably integrated into the cell.

An “exogenous” molecule is a molecule that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally-functioning endogenous molecule.

An exogenous molecule can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylases, demethylases, acetylases, deacetylases, kinases, phosphatases, ligases, deubiquitinases, integrases, recombinases, ligases, topoisomerases, gyrases and helicases.

An exogenous molecule can be the same type of molecule as an endogenous molecule, e.g., an exogenous protein or nucleic acid. For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer. An exogenous molecule can also be the same type of molecule as an endogenous molecule but derived from a different species than the cell is derived from. For example, a human nucleic acid sequence may be introduced into a cell line originally derived from a mouse or hamster. Methods for the introduction of exogenous molecules into plant cells are known to those of skill in the art and include, but are not limited to, protoplast transformation, silicon carbide (e.g., WHISKERS™), Agrobacterium-mediated transformation, lipid-mediated transfer (i.e., liposomes, including neutral and cationic lipids), electroporation, direct injection, cell fusion, particle bombardment (e.g., using a “gene gun”), calcium phosphate co-precipitation, DEAE-dextran-mediated transfer and viral vector-mediated transfer.

By contrast, an “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

As used herein, the term “product of an exogenous nucleic acid” includes both polynucleotide and polypeptide products, for example, transcription products (polynucleotides such as RNA) and translation products (polypeptides).

A “fusion” molecule is a molecule in which two or more subunit molecules are linked, preferably covalently. The subunit molecules can be the same chemical type of molecule, or can be different chemical types of molecules. Examples of fusion molecules include, but are not limited to, fusion proteins (for example, a fusion between a protein DNA-binding domain and a cleavage domain), fusions between a polynucleotide DNA-binding domain (e.g., sgRNA) operatively associated with a cleavage domain, and fusion nucleic acids (for example, a nucleic acid encoding the fusion protein).

Expression of a fusion protein in a cell can result from delivery of the fusion protein to the cell or by delivery of a polynucleotide encoding the fusion protein to a cell, wherein the polynucleotide is transcribed, and the transcript is translated, to generate the fusion protein. Trans-splicing, polypeptide cleavage and polypeptide ligation can also be involved in expression of a protein in a cell. Methods for polynucleotide and polypeptide delivery to cells are presented elsewhere in this disclosure.

A “gene,” for the purposes of the present disclosure, includes a DNA region encoding a gene product (see infra), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information contained in a gene, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” of gene expression refers to a change in the activity of a gene. Modulation of expression can include, but is not limited to, gene activation and gene repression. Genome editing (e.g., cleavage, alteration, inactivation, random mutation) can be used to modulate expression. Gene inactivation refers to any reduction in gene expression as compared to a cell that does not include a ZFP system as described herein. Thus, gene inactivation may be partial or complete.

A “region of interest” is any region of cellular chromatin, such as, for example, a gene or a non-coding sequence within or adjacent to a gene, in which it is desirable to bind an exogenous molecule. Binding can be for the purposes of targeted DNA cleavage and/or targeted recombination. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs.

A “reporter gene” or “reporter sequence” refers to any sequence that produces a protein product that is easily measured, preferably although not necessarily in a routine assay. Suitable reporter genes include, but are not limited to, sequences encoding proteins that mediate antibiotic resistance (e.g., ampicillin resistance, neomycin resistance, G418 resistance, puromycin resistance), sequences encoding colored or fluorescent or luminescent proteins (e.g., green fluorescent protein, enhanced green fluorescent protein, red fluorescent protein, luciferase), and proteins which mediate enhanced cell growth and/or gene amplification (e.g., dihydrofolate reductase). Epitope tags include, for example, one or more copies of FLAG, His, myc, Tap, HA or any detectable amino acid sequence. “Expression tags” include sequences that encode reporters that may be operably linked to a desired gene sequence in order to monitor expression of the gene of interest.

“Eukaryotic” cells include, but are not limited to, fungal cells (such as yeast), plant cells, animal cells, mammalian cells and human cells (e.g., T-cells), including stem cells (pluripotent and multipotent).

The terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

A “functional fragment” of a protein, polypeptide or nucleic acid is a protein, polypeptide or nucleic acid whose sequence is not identical to the full-length protein, polypeptide or nucleic acid, yet retains the same function as the full-length protein, polypeptide or nucleic acid. A functional fragment can possess more, fewer, or the same number of residues as the corresponding native molecule, and/or can contain one or more amino acid or nucleotide substitutions. Methods for determining the function of a nucleic acid or protein (e.g., coding function, ability to hybridize to another nucleic acid, enzymatic activity assays) are well-known in the art.

A polynucleotide “vector” or “construct” is capable of transferring gene sequences to target cells. Typically, “vector construct,” “expression vector,” “expression construct,” “expression cassette,” and “gene transfer vector,” mean any nucleic acid construct capable of directing the expression of a gene of interest and which can transfer gene sequences to target cells. Thus, the term includes cloning, and expression vehicles, as well as integrating vectors.

The terms “subject” and “patient” are used interchangeably and refer to mammals such as human patients and non-human primates, as well as experimental animals such as rabbits, dogs, cats, rats, mice, and other animals. Accordingly, the term “subject” or “patient” as used herein means any mammalian patient or subject to which the expression cassettes of the invention can be administered. Subjects of the present invention include those with a disorder.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Cancer, monogenic diseases and graft versus host disease are non-limiting examples of conditions that may be treated using the compositions and methods described herein.

A “target site” or “target sequence” is a nucleic acid sequence that defines a portion of a nucleic acid to which a binding molecule will bind, provided sufficient conditions for binding exist. For example, the sequence 5′-GAATTC-3′ is a target site for the Eco RI restriction endonuclease. An “intended” or “on-target” sequence is the sequence to which the binding molecule is intended to bind and an “unintended” or “off-target” sequence includes any sequence bound by the binding molecule that is not the intended target.

As used herein, the term “nuclease” refers to an enzyme which possesses catalytic activity for DNA cleavage. Any nuclease agent that induces a nick or double-strand break into a desired recognition site can be used in the methods and compositions disclosed herein. A naturally-occurring or native nuclease agent can be employed so long as the nuclease agent induces a nick or double-strand break in a desired recognition site. Alternatively, a modified or engineered nuclease agent can be employed. An “engineered nuclease agent” comprises a nuclease that is engineered (modified or derived) from its native form to specifically recognize and induce a nick or double-strand break in the desired recognition site. Thus, an engineered nuclease agent can be derived from a native, naturally-occurring nuclease agent or it can be artificially created or synthesized. The modification of the nuclease agent can be as little as one amino acid in a protein cleavage agent or one nucleotide in a nucleic acid cleavage agent. In some aspects, the engineered nuclease induces a nick or double-strand break in a recognition site, wherein the recognition site was not a sequence that would have been recognized by a native (non-engineered or non-modified) nuclease agent. Producing a nick or double-strand break in a recognition site or other DNA can be referred to herein as “cutting” or “cleaving” the recognition site or other DNA.

“Complement” or “complementary” as used herein refers to Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

### III. Zinc Finger Nuclease

The present disclosure is directed to a zinc finger nuclease (ZFN) that cleaves a CIITA gene, wherein the ZFN comprises a Zinc Finger DNA-binding domain that binds to a sequence in the CIITA gene and a cleavage domain. A zinc finger nuclease that cleaves a CIITA gene can be used to generate a cell that does not express or has a reduced expression of the CIITA gene. In some aspects, a ZFN can form a pair with another ZFN to cleave a site in a CIITA gene.

A protein known as CIITA (class II transactivator) which is a non-DNA binding protein, serves as a master control factor for MHC class II expression. In contrast to the other enhanceosome members, CIITA does exhibit tissue specific expression, is up-regulated by IFN-γ, and has been shown to be inhibited by several bacteria and viruses which can cause a down regulation of MHC class II expression (thought to be part of a bacterial attempt to evade immune surveillance (see LeibundGut-Landmann et al (2004) Eur. J. Immunol 34:1513-1525)). The CIITA protein is located in the nucleus and acts as a master regulator for the expression of MHC class II genes. MHC class II proteins are found on the surface of several types of immune cells and play a key role in the body's immune response against foreign invaders. Therefore, without wishing to be bound by theory, knocking down or knocking out CIITA gene function can improve the efficacy of allogeneic cell therapies by minimizing rejection by the host.

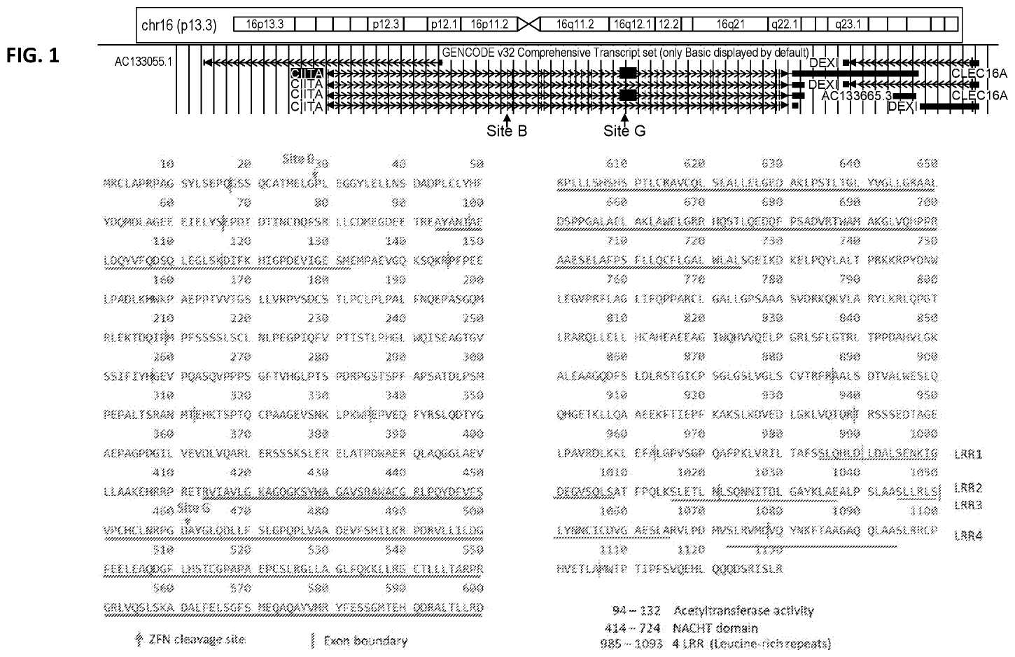

In humans, the CIITA protein is encoded by the CIITA gene, which is located on chromosome 16 (nucleotides 10,866,208 to 10,941,562 of GenBank Accession No. NC_000016.10). The CIITA gene spans 59191 bp on the short arm of chromosome 16 and encompasses 20 exons (FIG. 1). Synonyms of the CIITA gene, and encoded protein thereof, are known and include “C2TA,” “NLRA,” “MHC2TA,” “CIITAIV,” “MHC Class II Transactivator,” “Nucleotide-Binding Oligomerization Domain, Leucine Rich Repeat And Acid Domain Containing,” “NLR Family, Acid Domain Containing,” “Class II, Major Histocompatibility Complex, Transactivator,” “MHC Class II Transactivator Type III,” “MHC Class II Transactivator Type I,” “EC 2.7.11.1,” and “EC 2.3.1.” The CIITA gene can have four isoforms derived from alternative splicing. The isoform 1 encodes an 1130 amino acid protein which is considered the canonical sequence, shown in Table 1.

TABLE 1
CIITA protein sequences

| | |
|---|---|
| CIITA | MRCLAPRPAGSYLSEPQGSSQCATMELGPLEGGYLELLNSDADPLCLYHFYDQMDLAGEE |
| Isoform 1 (UniProt | EIELYSEPDTDTINCDQFSRLLCDMEGDEETREAYANIAELDQYVFQDSQLEGLSKDIFK |
| identifier: | HIGPDEVIGESMEMPAEVGQKSQKRPFPEELPADLKHWKPAEPPTVVTGSLLVRPVSDCS |
| P33076-1) | TLPCLPLPALFNQEPASGQMRLEKTDQIPMPFSSSSLSCLNLPEGPIQFVPTISTLPHGL |
| (SEQ ID NO: 1) | WQISEAGTGVSSIFIYHGEVPQASQVPPPSGFTVHGLPTSPDRPGSTSPFAPSATDLPSM |
| | PEPALTSRANMTEHKTSPTQCPAAGEVSNKLPKWPEPVEQFYRSLQDTYGAEPAGPDGIL |
| | VEVDLVQARLERSSSKSLERELATPDWAERQLAQGGLAEVLLAAKEHRRPRETRVIAVLG |
| | KAGQGKSYWAGAVSRAWACGRLPQYDFVFSVPCHCLNRPGDAYGLQDLLFSLGPQPLVAA |
| | DEVFSHILKRPDRVLLILDGFEELEAQDGFLHSTCGPAPAEPCSLRGLLAGLFQKKLLRG |
| | CTLLLTARPRGRLVQSLSKADALFELSGFSMEQAQAYVMRYFESSGMTEHQDRALTLLRD |
| | RPLLLSHSHSPTLCRAVCQLSEALLELGEDAKLPSTLTGLYVGLLGRAALDSPPGALAEL |
| | AKLAWELGRRHQSTLQEDQFPSADVRTWAMAKGLVQHPPRAAESELAFPSFLLQCFLGAL |
| | WLALSGEIKDKELPQYLALTPRKKRPYDNWLEGVPRFLAGLIFQPPARCLGALLGPSAAA |
| | SVDRKQKVLARYLKRLQPGTLRARQLLELLHCAHEAEEAGIWQHVVQELPGRLSFLGTRL |
| | TPPDAHVLGKALEAAGQDFSLDLRSTGICPSGLGSLVGLSCVTRFRAALSDTVALWESLQ |
| | QHGETKLLQAAEEKFTIEPFKAKSLKDVEDLGKLVQTQRTRSSSEDTAGELPAVRDLKKL |
| | EFALGPVSGPQAFPKLVRILTAFSSLQHLDLDALSENKIGDEGVSQLSATFPQLKSLETL |
| | NLSQNNITDLGAYKLAEALPSLAASLLRLSLYNNCICDVGAESLARVLPDMVSLRVMDVQ |
| | YNKFTAAGAQQLAASLRRCPHVETLAMWTPTIPFSVQEHLQQQDSRISLR |
| CIITA | MRCLAPRPAGSYLSEPQGSSQCATMELGPLEGGYLELLNSDADPLCLYHFYDQMDLAGEE |
| Isoform 2 | EIELYSEPDTDTINCDQFSRLLCDMEGDEETREAYANIAELDQYVFQDSQLEGLSKDIFK |
| (identifier: | HIGPDEVIGESMEMPAEVGQKSQKRPFPEELPADLKHWKPVPFSSSSLSCLNLPEGPIQF |
| P33076-2) | VPTISTLPHGLWQISEAGTGVSSIFIYHGEVPQASQVPPPSGFTVHGLPTSPDRPGSTSP |
| (SEQ ID NO: 2) | FAPSATDLPSMPEPALTSRANMTEHKTSPTQCPAAGEVSNKLPKWPEPVEQFYRSLQDTY |
| | GAEPAGPDGILVEVDLVQARLERSSSKSLERELATPDWAERQLAQGGLAEVLLAAKEHRR |
| | PRETRVIAVLGKAGQGKSYWAGAVSRAWACGRLPQYDFVFSVPCHCLNRPGDAYGLQDLL |
| | FSLGPQPLVAADEVFSHILKRPDRVLLILDGFEELEAQDGFLHSTCGPAPAEPCSLRGLL |
| | AGLFQKKLLRGCTLLLTARPRGRLVQSLSKADALFELSGFSMEQAQAYVMRYFESSGMTE |
| | HQDRALTLLRDRPLLLSHSHSPTLCRAVCQLSEALLELGEDAKLPSTLTGLYVGLLGRAA |
| | LDSPPGALAELAKLAWELGRRHQSTLQEDQFPSADVRTWAMAKGLVQHPPRAAESELAFP |
| | SFLLQCFLGALWLALSGEIKDKELPQYLALTPRKKRPYDNWLEGVPRFLAGLIFQPPARC |
| | LGALLGPSAAASVDRKQKVLARYLKRLQPGTLRARQLLELLHCAHEAEEAGIWQHVVQEL |
| | PGRLSFLGTRLTPPDAHVLGKALEAAGQDFSLDLRSTGICPSGLGSLVGLSCVTRFRWGE |
| | GLGRDILVLGINCGLGAKPSALWGPFSMQSSRVGQNGFSPFLR |
| CIITA | MRCLAPRPAGSYLSEPQGSSQCATMELGPLEGGYLELLNSDADPLCLYHFYDQMDLAGEE |
| Isoform 3 | EIELYSEPDTDTINCDQFSRLLCDMEGDEETREAYANIAELDQYVFQDSQLEGLSKDIFK |
| (identifier: | HIGPDEVIGESMEMPAEVGQKSQKRPFPEELPADLKHWKPVPFSSSSLSCLNLPEGPIQF |
| P33076-3) | VPTISTLPHGLWQISEAGTGVSSIFIYHGEVPQASQVPPPSGFTVHGLPTSPDRPGSTSP |
| (SEQ ID NO: 3) | FAPSATDLPSMPEPALTSRANMTEHKTSPTQCPAAGEVSNKLPKWPGLAWSPCLGLRPSL |
| | HRAALSDTVALWESLQQHGETKLLQAAEEKFTIEPFKAKSLKDVEDLGKLVQTQRTRSSS |
| | EDTAGELPAVRDLKKLEFALGPVSGPQAFPKLVRILTAFSSLQHLDLDALSENKIGDEGV |
| | SQLSATFPQLKSLETLNLSQNNITDLGAYKLAEALPSLAASLLRLSLYNNCICDVGAESL |
| | ARVLPDMVSLRVMDVQYNKFTAAGAQQLAASLRRCPHVETLAMWTPTIPFSVQEHLQQQD |
| | SRISLR |
| CIITA | MRCLAPRPAGSYLSEPQGSSQCATMELGPLEGGYLELLNSDADPLCLYHFYDQMDLAGEE |
| Isoform 4 | EIELYSEPDTDTINCDQFSRLLCDMEGDEETREAYANIAELDQYVFQDSQLEGLSKDIFK |
| (identifier: | HIGPDEVIGESMEMPAEVGQKSQKRPFPEELPADLKHWKPAEPPTVVTGSLLVRPVSDCS |
| P33076-4) | TLPCLPLPALFNQEPASGQMRLEKTDQIPMPFSSSSLSCLNLPEGPIQFVPTISTLPHGL |
| (SEQ ID NO: 4) | WQISEAGTGVSSIFIYHGEVPQASQVPPPSGFTVHGLPTSPDRPGSTSPFAPSATDLPSM |
| | PEPALTSRANMTEHKTSPTQCPAAGEVSNKLPKWPEPVEQFYRSLQDTYGAEPAGPDGIL |
| | VEVDLVQARLERSSSKSLERELATPDWAERQLAQGGLAEVLLAAKEHRRPRETRVIAVLG |
| | KAGQGKSYWAGAVSRAWACGRLPQYDFVFSVPCHCLNRPGDAYGLQDLLFSLGPQPLVAA |
| | DEVFSHILKRPDRVLLILDGFEELEAQDGFLHSTCGPAPAEPCSLRGLLAGLFQKKLLRG |
| | CTLLLTARPRGRLVQSLSKADALFELSGFSMEQAQAYVMRYFESSGMTEHQDRALTLLRD |
| | RPLLLSHSHSPTLCRAVCQLSEALLELGEDAKLPSTLTGLYVGLLGRAALDSPPGALAEL |
| | AKLAWELGRRHQSTLQEDQFPSADVRTWAMAKGLVQHPPRAAESELAFPSFLLQCFLGAL |
| | WLALSGEIKDKELPQYLALTPRKKRPYDNWLEGVPRFLAGLIFQPPARCLGALLGPSAAA |
| | SVDRKQKVLARYLKRLQPGTLRARQLLELLHCAHEAEEAGIWQHVVQELPGRLSFLGTRL |
| | TPPDAHVLGKALEAAGQDFSLDLRSTGICPSGLGSLVGLSCVTRFRWGEGLGRDILVLGI |
| | NCGLGAKPSALWGPFSMQSSRVGQNGFSPFLR |

In some aspects, the zinc finger nuclease can target one or more sites in the CIITA gene. In some aspects, the zinc finger nuclease cleaving a DNA sequence in the CIITA gene is between amino acid 26 and amino acid 32, e.g., amino acids 28 and 29 corresponding to SEQ ID NO: 1. In some aspects, the zinc finger nuclease cleaving a DNA sequence in the CIITA gene is between amino acid 457 and amino acid 465, e.g., amino acids 461 and 462 corresponding to SEQ ID NO: 1.

In some aspects, a ZFN of the present disclosure comprises an amino acid sequence having at least about 70%, at least about 80%, at least about 85%, at least about 90%, at least about 95%, at least about 96%, at least about 97%, at least about 98%, at least about 99%, or about 100% sequence identity to SEQ ID NO: 5

(MDYKDHDGDYKDHDIDYKDDDDKMAPKKKRKVGIHGVPAAMGQL
VKSELEEKKSELRHKLKYVPHEYIELIEIARNSTQDRILEMKVME
FFMKVYGYRGKHLGGSRKPDGAIYTVGSPIDYGVIVDTKAYSGGY
NLPIGQADEMERYVEENQTRDKHLNPNEWWKVYPSSVTEFKFLFV
SGHFKGNYKAQLTRLNHITNCNGAVLSVEELLIGGEMIKAGTLTL
EEVRRKFNNGEINFSGAQGSTLDFRPFQCRICMRNFSRPYTLRLH
IRTHTGEKPFACDICGRKFARSANLTRHTKIHTGSQKPFQCRICM
RNFSRSDALSTHIRTHTGEKPFACDICGRKFADRSTRTKHTKIHT
GEKPFQCRICMRKFADRSTRTKHTKIHLRQKD).

In some aspects, a ZFN of the present disclosure comprises an amino acid sequence having at least about 70%, at least about 80%, at least about 85%, at least about 90%, at least about 95%, at least about 96%, at least about 97%, at least about 98%, at least about 99%, or about 100% sequence identity to SEQ ID NO: 6

(MDYKDHDGDYKDHDIDYKDDDDKMAPKKKRKVGIHGVPAAMAER
PFQCRICMQNFSRSDVLSAHIRTHTGEKPFACDICGKKFADRSNR
IKHTKIHTGSQKPFQCRICMQNFSDRSHLTRHIRTHTGEKPFACD
ICGRKFALKQHLTRHTKIHTGEKPFQCRICMQNFSQSGNLARHIR
THTGEKPFACDICGRKFAQSTPRTTHTKIHLRGSQLVKSELEEKK
SELRHKLKYVPHEYIELIEIARNSTQDRILEMKVMEFFMKVYGYR
GKHLGGSRKPDGAIYTVGSPIDYGVIVDTKAYSGGYNLPIGQADE
MQRYVKENQTRNKHINPNEWWKVYPSSVTEFKFLFVSGHFKGNYK
AQLTRLNRKTNCNGAVLSVEELLIGGEMIKAGTLTLEEVRRKFNN
GEINF).

In some aspects, a ZFN pair of the present disclosure comprises a first ZFN comprising an amino acid sequence having at least about 70%, at least about 80%, at least about 85%, at least about 90%, at least about 95%, at least about 96%, at least about 97%, at least about 98%, at least about 99%, or about 100% sequence identity to SEQ ID NO: 5 (MDYKDHDGDYKDHDIDYKDDDDKMAPKKKRKVGIHGVPAAMGQLVKSELEEKKSELRHKLKYVPHEYIELIEIARNST QDRILEMKVMEFFMKVYGYRGKHLGGSRKPDGAIYTVGSPIDYGVIVDTKAYSGGYNLPIGQADEMERYVEENQTRDK HLNPNEWWKVYPSSVTEFKFLFVSGHFKGNYKAQLTRLNHITNCNGAVLSVEELLIGGEMIKAGTLTLEEVRRKENNG EINFSGAQGSTLDFRPFQCRICMRNFSRPYTLRLHIRTHTGEKPFACDICGRKFARSANLTRHTKIHTGSQKPFQCRI CMRNFSRSDALSTHIRTHTGEKPFACDICGRKFADRSTRTKHTKIHTGEKPFQCRICMRKFADRSTRTKHTKIHLROK D) and a second ZFN comprising an amino acid sequence having at least about 70%, at least about 80%, at least about 85%, at least about 90%, at least about 95%, at least about 96%, at least about 97%, at least about 98%, at least about 99%, or about 100% sequence identity to SEQ ID NO: 6

(MDYKDHDGDYKDHDIDYKDDDDKMAPKKKRKVGIHGVPAAMAERPFQC
RICMQNFSRSDVLSAHIRTHTGEKPFACDICGKKFADRSNRIKHTKIHT
GSQKPFQCRICMQNFSDRSHLTRHIRTHTGEKPFACDICGRKFALKQHL
TRHTKIHTGEKPFQCRICMQNFSQSGNLARHIRTHTGEKPFACDICGRK
FAQSTPRTTHTKIHLRGSQLVKSELEEKKSELRHKLKYVPHEYIELIEI
ARNSTQDRILEMKVMEFFMKVYGYRGKHLGGSRKPDGAIYTVGSPIDYG
VIVDTKAYSGGYNLPIGQADEMQRYVKENQTRNKHINPNEWWKVYPSSV
TEFKFLFVSGHFKGNYKAQLTRLNRKTNCNGAVLSVEELLIGGEMIKAG
TLTLEEVRRKFNNGEINF).

In some aspects, a ZFN of the present disclosure comprises an amino acid sequence having at least about 70%, at least about 80%, at least about 85%, at least about 90%, at least about 95%, at least about 96%, at least about 97%, at least about 98%, at least about 99%, or about 100% sequence identity to SEQ ID NO: 7

(MDYKDHDGDYKDHDIDYKDDDDKMAPKKKRKVGIHGVPAAMGQLVKSE
LEEKKSELRHKLKYVPHEYIELIEIARNSTQDRILEMKVMEFFMKVYGY
RGKHLGGSRKPDGAIYTVGSPIDYGVIVDTKAYSGGYNLPIGQADEMER
YVEENQTRDKHLNPNEWWKVYPSSVTEFKFLFVSGHFKGNYKAQLTRLN
HITNCNGAVLSVEELLIGGEMIKAGTLTLEEVRRKFNNGEINFSGTPHE
VGVYTLRPFQCRICMRNFSRSDHLSRHIRTHTGEKPFACDICGRKFADS
SDRKKHTKIHTGEKPFQCRICMRNFSRSDTLSEHIRTHTGEKPFACDIC
GRKFAQSGDLTRHTKIHTHPRAPIPKPFQCRICMRNFSQSSDLSRHIRT
HTGEKPFACDICGRKFAYKWTLRNHTKIHLRQKD)
or
SEQ ID NO: 54
(MDYKDHDGDYKDHDIDYKDDDDKMAPKKKRKVGIHGVPAAMGQLVKSE
LEEKKSELRHKLKYVPHEYIELIEIARNSTQDRILEMKVMEFFMKVYGY
RGKHLGGSRKPDGAIYTVGSPIDYGVIVDTKAYSGGYNLPIGQADEMER
YVEENQTRDKHLNPNEWWKVYPSSVTEFKFLFVSGHFSGNYKAQLTRLN
HITNCNGAVLSVEELLIGGEMIKAGTLTLEEVRRKENNGEINFSGTPHE
VGVYTLRPFQCRICMRNFSRSDHLSRHIRTHTGEKPFACDICGRKFADS
SDRKKHTKIHTGEKPFQCRICMRNFSRSDTLSEHIRTHTGEKPFACDIC
GRKFAQSGDLTRHTKIHTHPRAPIPKPFQCRICMRNFSQSSDLSRHIRT
HTGEKPFACDICGRKFAYKWTLRNHTKIHLRQKD).

In some aspects, a ZFN of the present disclosure comprises an amino acid sequence having at least about 70%, at least about 80%, at least about 85%, at least about 90%, at least about 95%, at least about 96%, at least about 97%, at least about 98%, at least about 99%, or about 100% sequence identity to SEQ ID NO: 8

(MDYKDHDGDYKDHDIDYKDDDDKMAPKKKRKVGIHGVPAAMGQLVKSE
LEEKKSELRHKLKYVPHEYIELIEIARNSTQDRILEMKVMEFFMKVYGY
RGKHLGGSRKPDGAIYTVGSPIDYGVIVDTKAYSGGYNLPIGQADEMQR
YVKENQTRNKHINPNEWWKVYPSSVTEFKFLFVSGHFKGNYKAQLTRLN
RKTNCNGAVLSVEELLIGGEMIKAGTLTLEEVRRKENNGEINFSGTPHE
VGVYTLRPFQCRICMRNFSSNQNLTTHIRTHTGEKPFACDICGRKFADR
SHLARHTKIHTGEKPFQCRICMQKFAQSGDLTRHTKIHTGEKPFQCRIC
MQNFSWKHDLTNHIRTHTGEKPFACDICGRKFATSGNLTRHTKIHLRQK
D)
or
SEQ ID NO: 56
(QLVKSELEEKKSELRHKLKYVPHEYIELIEIARNSTQDRILEMKVMEF
FMKVYGYRGKHLGGSRKPDGAIYTVGSPIDYGVIVDTKAYSGGYNLPTG
QADEMQRYVKENQTRNKHINPNEWWKVYPSSVTEFKFLFVSGHFKGNYK
AQLTRLNRKTNCNGAVLSVEELLIGGEMIKAGTLTLEEVRRKFNNGEIN
FSGTPHEVGVYTLRPFQCRICMRNFSSNQNLTTHIRTHTGEKPFACDIC
GRKFADRSHLARHTKIHTGEKPFQCRICMQKFAQSGDLTRHTKIHTGEK
PFQCRICMQNFSWKHDLTNHIRTHTGEKPFACDICGRKFATSGNLTRHT
KIHLRQKD).

In some aspects, a ZFN pair of the present disclosure comprises a first ZFN comprising an amino acid sequence having at least about 70%, at least about 80%, at least about 85%, at least about 90%, at least about 95%, at least about 96%, at least about 97%, at least about 98%, at least about 99%, or about 100% sequence identity to SEQ ID NO: 7 (

MDYKDHDGDYKDHDIDYKDDDDKMAPKKKRKVGIHGVPAAMGQLVKSELEEKKSELRHKLKYVPHE YIELIEIARNSTQDRILEMKVMEFFMKVYGYRGKHLGGSRKPDGAIYTVGSPIDYGVIVDTKAYSGGYNLPIGQADEM ERYVEENQTRDKHLNPNEWWKVYPSSVTEFKFLFVSGHFKGNYKAQLTRLNHITNCNGAVLSVEELLIGGEMIKAGTL TLEEVRRKFNNGEINFSGTPHEVGVYTLRPFQCRICMRNFSRSDHLSRHIRTHTGEKPFACDICGRKFADSSDRKKHT KIHTGEKPFQCRICMRNFSRSDTLSEHIRTHTGEKPFACDICGRKFAQSGDLTRHTKIHTHPRAPIPKPFQCRICMRN FSQSSDLSRHIRTHTGEKPFACDICGRKFAYKWTLRNHTKIHLROKD) and a second ZFN comprising an amino acid sequence having at least about 70%, at least about 80%, at least about 85%, at least about 90%, at least about 95%, at least about 96%, at least about 97%, at least about 98%, at least about 99%, or about 100%8 sequence identity to SEQ ID NO:

(MDYKDHDGDYKDHDIDYKDDDDKMAPKKKRKVGIHGVPAAMGQLVKSE
LEEKKSELRHKLKYVPHEYIELIEIARNSTQDRILEMKVMEFFMKVYGY
RGKHLGGSRKPDGAIYTVGSPIDYGVIVDTKAYSGGYNLPIGQADEMQR
YVKENQTRNKHINPNEWWKVYPSSVTEFKFLFVSGHFKGNYKAQLTRLN
RKTNCNGAVLSVEELLIGGEMIKAGTLTLEEVRRKFNNGEINFSGTPHE
VGVYTLRPFQCRICMRNFSSNQNLTTHIRTHTGEKPFACDICGRKFADR
SHLARHTKIHTGEKPFQCRICMQKFAQSGDLTRHTKIHTGEKPFQCRIC
MQNFSWKHDLTNHIRTHTGEKPFACDICGRKFATSGNLTRHTKIHLRQK
D).

In some aspects, a ZFN pair of the present disclosure comprises a first ZFN comprising an amino acid sequence having at least about 70%, at least about 80%, at least about 85%, at least about 90%, at least about 95%, at least about 96%, at least about 97%, at least about 98%, at least about 99%, ID NO: 54 or about 100% sequence identity to SEQ (MDYKDHDGDYKDHDIDYKDDDDKMAPKKKRKVGIHGVPAAMGOLVKSELEEKKSELRHKLKYVPHEYIELIEIARNS TQDRILEMKVMEFFMKVYGYRGKHLGGSRKPDGAIYTVGSPIDYGVIVDTKAYSGGYNLPIGQADEMERYVEENQTRD KHLNPNEWWKVYPSSVTEFKFLFVSGHFSGNYKAQLTRINHITNCNGAVLSVEELLIGGEMIKAGTLTLEEVRRKENN GEINFSGTPHEVGVYTLRPFQCRICMRNFSRSDHLSRHIRTHTGEKPFACDICGRKFADSSDRKKHTKIHTGEKPFQC RICMRNFSRSDTLSEHIRTHTGEKPFACDICGRKFAQSGDLTRHTKIHTHPRAPIPKPFQCRICMRNFSQSSDLSRHI RTHTGEKPFACDICGRKFAYKWTLRNHTKIHLROKD) and a second ZFN comprising an amino acid sequence having at least about 70%, at least about 80%, at least about 85%, at least about 90%, at least about 95%, at least about 96%, at least about 97%, at least about 98%, at least about 99%, or about NO: 100% sequence identity to SEQ ID 56 (QLVKSELEEKKSELRHKLKYVPHEYIELIEIARNSTQDRILEMKVMEFFMKVYGYRGKHLGGSRKPDGAIYTVGSPI DYGVIVDTKAYSGGYNLPTGQADEMQRYVKENQTRNKHINPNEWWKVYPSSVTEFKFLFVSGHFKGNYKAQLTRLNRK TNCNGAVLSVEELLIGGEMIKAGTLTLEEVRRKFNNGEINFSGTPHEVGVYTLRPFQCRICMRNFSSNQNLTTHIRTH TGEKPFACDICGRKFADRSHLARHTKIHTGEKPFQCRICMQKFAQSGDLTRHTKIHTGEKPFQCRICMQNFSWKHDLT NHIRTHTGEKPFACDICGRKFATSGNLTRHTKIHLROKD). In some aspects, the first ZFN and the second ZFN are linked. In some aspects, the linkage between the first ZFN and the second ZFN is a peptide linker. In some aspects, the linkage between the first ZFN and the second ZFN is a cleavable linker. In some aspects, the linker comprises a P2A linker, a T2A linker, or any combination thereof.

### IIIA. Zinc Finger DNA-Binding Domains

Described herein are DNA-binding domains that specifically bind to a DNA sequence in a CIITA gene and polynucleotides encoding the same. In some aspects, the DNA-binding domains comprises five or more zinc fingers. Engineered zinc finger binding domains can have a novel binding specificity, compared to a naturally-occurring zinc finger protein.

In some aspects, the zinc finger nuclease is capable of cleaving a CIITA gene between amino acid 26 and amino acid 30, e.g., amino acid 28 and amino acid 29 corresponding to SEQ ID NO: 1. In some aspects, the zinc finger nuclease is capable of cleaving the CIITA gene between amino acid 457 and amino acid 465, e.g., amino acid 461 and amino acid 462 corresponding to SEQ ID NO: 1.

In some aspects, the DNA binding domain is capable of binding to GCCACCATGGAGTTG (SEQ ID NO: 9). In some aspects, the DNA binding domain that binds to GCCACCATGGAGTTG (SEQ ID NO: 9) has five zinc fingers: finger 1 (F1) comprises or consists of SEQ ID NO: 10 [RPYTLRL], finger 2 (F2) comprises or consists of SEQ ID NO: 11 [RSANLTR], finger 3 (F3) comprises or consists of SEQ ID NO: 12 [RSDALST], finger 4 (F4) comprises or consists of SEQ ID NO: 13 [DRSTRTK], and finger 5 (F5) comprises or consists of SEQ ID NO: 14 [DRSTRTK].

In some aspects, the DNA binding domain is capable of binding to CTAGAAGGTGGCTACCTG (SEQ ID NO: 15). In some aspects, the DNA binding domain that binds to CTAGAAGGTGGCTACCTG (SEQ ID NO: 15) comprises six zinc fingers: F1 comprises or consists of SEQ ID NO: 16 [RSDVLSA], F2 comprises or consists of SEQ ID NO: 17 [DRSNRIK], F3 comprises or consists of SEQ ID NO: 18 [DRSHLTR], F4 comprises or consists of SEQ ID NO: 19 [LKQHLTR], F5 comprises or consists of SEQ ID NO: 20 [QSGNLAR], and F6 comprises or consists of SEQ ID NO: 21 [QSTPRTT].

In some aspects, the DNA binding domain is capable of binding to ATTGCT and/or GAACCGTCCGGG (SEQ ID NO: 38). In some aspects, the DNA binding domain that binds to ATTGCT and/or GAACCGTCCGGG (SEQ ID NO: 38) has six zinc fingers: F1 comprises SEQ ID NO: 23 [RSDHLSR], F2 comprises SEQ ID NO: 24 [DSSDRKK], F3 comprises SEQ ID NO: 25 [RSDTLSE], F4 comprises 26 [QSGDLTR], and F5 comprises SEQ ID NO: 27 [QSSDLSR], and F6 comprises SEQ ID NO: 28 [YKWTLRN].

In some aspects, the DNA binding domain is capable of binding to GATCCTGCAGGCCAT (SEQ ID NO: 29). In some aspects, the DNA binding domain that binds to GATCCTGCAGGCCAT (SEQ ID NO: 29) comprises five-fingers: F1 comprises SEQ ID NO: 30 [SNQNLTT], F2 comprises SEQ ID NO: 31 [DRSHLAR], F3 comprises SEQ ID NO: 32 [QSGDLTR], F4 comprises SEQ ID NO: 33 [WKHDLTN], and F5 comprises SEQ ID NO: 34 [TSGNLTR].

In some aspects, a zinc finger nuclease pair cleaves a DNA sequence in a CIITA gene. In some aspects, the zinc finger pair is capable of cleaving the CIITA gene between amino acid 26 and amino acid 30, e.g., amino acid 28 and amino acid 29 corresponding to SEQ ID NO: 1. In some aspects, the zinc finger nuclease pair comprises a first zinc finger nuclease comprising finger 1 (F1) comprises or consists of SEQ ID NO: 10 [RPYTLRL], finger 2 (F2) comprises or consists of SEQ ID NO: 11 [RSANLTR], finger 3 (F3) comprises or consists of SEQ ID NO: 12 [RSDALST], finger 4 (F4) comprises or consists of 13[DRSTRTK], and finger 5 (F5) comprises or consists of SEQ ID NO: 14 [DRSTRTK] and a second zinc finger nuclease comprising F1 comprises or consists of SEQ ID NO: 16 [RSDVLSA], F2 comprises or consists of SEQ ID NO: 17 [DRSNRIK], F3 comprises or consists of SEQ ID NO: 18 [DRSHLTR], F4 comprises or consists of SEQ ID NO:19 [LKQHLTR], F5 comprises or consists of SEQ ID NO: 20 [QSGNLAR], and F6 comprises or consists of SEQ ID NO: 21 [QSTPRTT].

In some aspects, the zinc finger nuclease pair cleaves a CIITA gene between amino acid 457 and amino acid 465, e.g., amino acid 461 and amino acid 462 corresponding to SEQ ID NO: 1. In some aspects, the zinc finger nuclease pair comprises a first zinc finger nuclease comprising F1 comprises SEQ ID NO: 23 [RSDHLSR], F2 comprises SEQ ID NO: 24 [DSSDRKK], F3 comprises SEQ ID NO: 25 [RSDTLSE], F4 comprises SEQ ID NO: 26 [QSGDLTR], and F5 comprises SEQ ID NO: 27 [QSSDLSR], and F6 comprises SEQ ID NO: 28 [YKWTLRN] and a second zinc finger nuclease comprising F1 comprises SEQ ID NO: 30 [SNQNLTT], F2 comprises SEQ ID NO: 31 [DRSHLAR], F3 comprises SEQ ID NO: 32 [QSGDLTR], F4 comprises SEQ ID NO: 33 [WKHDLTN], and F5 comprises SEQ ID NO: 34 [TSGNLTR].

Non-limiting examples of the ZFNs are shown in Table 2.

TABLE 2

| | ZFN | Binding sequence | | | | | | | |
|---|---|---|---|---|---|---|---|---|---|
| Site | ID | (capital letter) | F1 | F2 | F3 | F4 | F5 | F6 | FokI |
| B | [symbol] | gtGCCACCATGGAGTTGgg | RPYTLRL | RSANLTR | RSDALST | DRSTRTK | DRSTRTK | | [symbol] |
| | [symbol] | ccCTAGAAGGTGGCTACCTGga | ^{∧}RSDVLSA | [symbol] | ^{∧}DRSHLTR | LKQHLTR | ^{∧}QSGNLAR | QSTPRTT | [symbol] |
| G | [symbol] | ccATTGCTtGAACCGTCCGGGgg | [symbol] | DSSDRKK | RSDTLSE | QSGDLTR | QSGDLSR | YKWTLRN | [symbol] |
| | [symbol] | ccGATCCTGCAGGCCATag | SNQNLTT | DRSHLAR | ^{∧}QSGDLTR | ^{∧}WKHDLTN | TSGNLTR | | [symbol] |

^{∧}The arginine residue at the 4^{th} position upstream of the 1^{st} amino acid in the indicated helix is changed to glutamine. [symbol]
indicates data missing or illegible when filed

Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, co-owned U.S. Pat. Nos. 6,453,242 and 6,534,261, incorporated by reference herein in their entireties.

Exemplary selection methods, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237. In addition, enhancement of binding specificity for zinc finger binding domains has been described, for example, in co-owned WO 02/077227.

In some aspects, zinc finger domains and/or multi-fingered zinc finger proteins can be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein can include any combination of suitable linkers between the individual zinc fingers of the protein. In addition, enhancement of binding specificity for zinc finger binding domains has been described, for example, in co-owned WO 02/077227.

Alternatively, the DNA-binding domain may be derived from a nuclease. For example, the recognition sequences of homing endonucleases and meganucleases such as I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII are known. See also U.S. Pat. Nos. 5,420,032; 6,833,252; Belfort et al. (1997) Nucleic Acids Res. 25:3379-3388; Dujon et al. (1989) Gene 82:115-118; Perler et al. (1994) Nucleic Acids Res. 22, 1125-1127; Jasin (1996) Trends Genet. 12:224-228; Gimble et al. (1996) J. Mol. Biol. 263: 163-180; Argast et al. (1998) J. Mol. Biol. 280:345-353 and the New England Biolabs catalogue. In addition, the DNA-binding specificity of homing endonucleases and meganucleases can be engineered to bind non-natural target sites. See, for example, Chevalier et al. (2002) Molec. Cell 10:895-905; Epinat et al. (2003) Nucleic Acids Res. 31:2952-2962; Ashworth et al. (2006) Nature 441:656-659; Paques et al. (2007) Current Gene Therapy 7:49-66; U.S. Patent Publication No. 20070117128.

### IIIB. Cleavage Domain

In addition, DNA binding domains have been fused to nuclease cleavage domains to create ZFNs—a functional entity that is able to recognize its intended nucleic acid target through its engineered ZFP DNA binding domain and cause DNA cleavage near the DNA binding site via the nuclease activity. See, e.g., Kim et al. (1996) Proc Nat'l Acad Sci USA 93(3):1156-1160. Therefore, in some aspects, the zinc finger nuclease further comprises a cleavage (nuclease) domain (e.g., FokI cleavage domain). More recently, such nucleases have been used for genome modification in a variety of organisms. See, for example, United States Patent Publications 20030232410; 20050208489; 20050026157; 20050064474; 20060188987; 20060063231; and International Publication WO 07/014275.

In some aspects, gene modification can be achieved using a nuclease, for example an engineered nuclease. Engineered nuclease technology is based on the engineering of naturally occurring DNA-binding proteins. For example, engineering of homing endonucleases with tailored DNA-binding specificities has been described. Chames et al. (2005) Nucleic Acids Res 33(20):e178; Arnould et al. (2006) J. Mol. Biol. 355:443-458. In addition, engineering of ZFPs has also been described. See, e.g., U.S. Pat. Nos. 6,534,261; 6,607,882; 6,824,978; 6,979,539; 6,933,113; 7,163,824; and 7,013,219.

In addition, as disclosed in these and other references, zinc finger domains, and zinc finger proteins may be linked together using any suitable linker sequences, including for example, linkers of 5 or more amino acids in length. See, e.g., U.S. Pat. Nos. 6,479,626; 6,903,185; and 7,153,949 for exemplary linker sequences 6 or more amino acids in length. The proteins described herein may include any combination of suitable linkers between the individual zinc fingers of the protein. See, also, U.S. Pat. No. 8,772,453.

In some aspects, the cleavage domains can be derived from any nuclease, or functional fragment thereof, that requires dimerization for cleavage activity. In some aspects, the cleavage domain dimers may be homodimers or heterodimers (e.g., derived from the same or different endonucleases, or differentially modified endonucleases).

Restriction endonucleases (restriction enzymes) are present in many species and are capable of sequence-specific binding to DNA (e.g., at a recognition site), and cleaving DNA at or near the site of binding. Certain restriction enzymes (e.g., Type IIS) cleave DNA at sites removed from the recognition site and have separable binding and cleavage domains. For example, the Type IIS enzyme FokI catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31, 978-31,982. In some aspects, fusion proteins (e.g., ZFNs disclosed herein) comprise the cleavage domain from at least one Type IIS restriction enzyme and one or more zinc finger binding domains, which may or may not be engineered.

An exemplary Type IIS restriction enzyme, whose cleavage domain is separable from the binding domain, is FokI. This particular enzyme is active as a dimer. Bitinaite et al. (1998) Proc. Natl. Acad. Sci. USA 95: 10,570-10,575. Thus, for targeted double-stranded cleavage and/or targeted replacement of cellular sequences using zinc finger-FokI fusions, two fusion proteins, each comprising a FokI cleavage domain (e.g., a monomer), can be used to reconstitute a catalytically active cleavage domain (e.g., through the formation of a homo or hetero-dimers). In some aspects, a single polypeptide molecule containing a zinc finger binding domain and two Fok I cleavage dimers can also be used. Parameters for targeted cleavage and targeted sequence alteration using zinc finger-FokI fusions are provided herein.

In some aspects, a cleavage domain can be any portion of a protein that retains cleavage activity, or that retains the ability to multimerize (e.g., dimerize) to form a functional cleavage domain.

Exemplary Type IIS restriction enzymes are described in International Publication WO 07/014275, incorporated herein in its entirety. Additional restriction enzymes also contain separable binding and cleavage domains, and these are contemplated by the present disclosure. See, for example, Roberts et al. (2003) Nucleic Acids Res. 31:418-420.

In some aspects, the cleavage domain comprises a cleavage domain from a FokI endonuclease. The full length FokI protein sequence is shown in Table 3.

TABLE 3
FokI protein (SEQ ID NO: 35)

MVSKIRTFGWVQNPGKFENLKRVVQVFDRNSKVHNEVKNIKIPTLVKES
KIQKELVAIMNQHDLIYTYKELVGTGTSIRSEAPCDAIIQATIADQGNK
KGYIDNWSSDGFLRWAHALGFIEYINKSDSFVITDVGLAYSKSADGSAI
EKEILIEAISSYPPAIRILTLLEDGQHLTKFDLGKNLGFSGESGFTSLP
EGILLDTLANAMPKDKGEIRNNWEGSSDKYARMIGGWLDKLGLVKQGKK
EFIIPTLGKPDNKEFISHAFKITGEGLKVLRRAKGSTKFTRVPKRVYWE
MLATNLTDKEYVRTRRALILEILIKAGSLKIEQIQDNLKKLGFDEVIET
IENDIKGLINTGIFIEIKGRFYQLKDHILQFVIPNRGVTKQLVKSELEE
KKSELRHKLKYVPHEYIELIEIARNSTQDRILEMKVMEFFMKVYGYRGK
HLGGSRKPDGAIYTVGSPIDYGVIVDTKAYSGGYNLPIGQADEMQRYVE
ENQTRNKHINPNEWWKVYPSSVTEFKFLFVSGHFKGNYKAQLTRLNHIT
NCNGAVLSVEELLIGGEMIKAGTLTLEEVRRKFNNGEINF

In some aspects, the cleavage domains derived from FokI comprise one or more amino acids that are different from the wild type FokI protein. In some aspects, the cleavage domain comprises the sequences in Table 4.

TABLE 4
FokIELD and FokIKKR sequences

| | |
|---|---|
| FokIELD (SEQ | QLVKSELEEKKSELRHKLKYVPHEYIELIEIA |
| ID NO: 36) | RNSTQDRILEMKVMEFFMKVYGYRGKHLGGSR |
| | KPDGAIYTVGSPIDYGVIVDTKAYSGGYNLPI |
| | GQADEMERYVEENQTRDKHLNPNEWWKVYPSS |
| | VTEFKFLFVSGHFKGNYKAQLTRLNHITNCNG |
| | AVLSVEELLIGGEMIKAGTLTLEEVRRKFNNG |
| | EINF |
| FokIKKR (SEQ | QLVKSELEEKKSELRHKLKYVPHEYIELIEIA |
| ID NO: 37) | RNSTQDRILEMKVMEFFMKVYGYRGKHLGGSR |
| | KPDGAIYTVGSPIDYGVIVDTKAYSGGYNLPI |
| | GQADEMQRYVKENQTRNKHINPNEWWKVYPSS |
| | VTEFKFLFVSGHFKGNYKAQLTRLNRKTNCNG |
| | AVLSVEELLIGGEMIKAGTLTLEEVRRKFNNG |
| | EINF |

In some aspects, the FokI protein useful for the present disclosure include amino acids that are different from the wild-type, insertions (of one or more amino acid residues) and/or deletions (of one or more amino acid residues). In some aspects, one or more of residues 414-426, 443-450, 467-488, 501-502, and/or 521-531 (numbered relative to full length sequence above) are different from the wild type sequence since these residues are located close to the DNA backbone in a molecular model of a ZEN bound to its target site described in Miller et al. ((2007) Nat Biotechnol 25:778-784). In some aspects, one or more residues at positions 416, 422, 447, 448, and/or 525 are different from the corresponding wild type sequence. In some aspects, the FokI protein useful for the disclosure comprises one or more amino acids different from the corresponding wild-type residues, for example an alanine (A) residue, a cysteine (C) residue, an aspartic acid (D) residue, a glutamic acid (E) residue, a histidine (H) residue, a phenylalanine (F) residue, a glycine (G) residue, an asparagine (N) residue, a serine (S) residue or a threonine (T) residue. In some aspects, the wild-type residue at one or more of positions 416, 418, 422, 446, 448, 476, 479, 480, 481, 525, 527 and/or 531 are replaced with any other residues, including but not limited to, R416E, R416F, R416N, S418D, S418E, R422H, N476D, N476E, N476G, N476T, I479T, I479Q, Q481A, Q481D, Q481E, Q481H, K525A, K525S, K525T, K525V, N527D, and/or Q531R. In some aspects, the wild-type residue lysine (K) at position 525 of SEQ ID NO: 35 is replaced with a serine (S) residue. In some aspects, the wild-type residue isoleucine (I) at position 479 of SEQ ID NO: 35 is replaced with a threonine (T) residue.

In some aspects, the cleavage domain comprises one or more engineered dimers (also referred to as dimerization domain mutants) that minimize or prevent homodimerization, as described, for example, in U.S. Pat. Nos. 7,914,796; 8,034,598 and 8,623,618; and U.S. Patent Publication No. 20110201055, the disclosures of all of which are incorporated by reference in their entireties herein. Amino acid residues at positions 446, 447, 479, 483, 484, 486, 487, 490, 491, 496, 498, 499, 500, 531, 534, 537, and 538 of FokI (numbered relative full length FokI sequence) are all targets for influencing dimerization of the FokI cleavage domains. The mutations can include mutations to residues found in natural restriction enzymes homologous to FokI. In some aspects, the mutation at positions 416, 422, 447, 448 and/or 525 comprise replacement of a positively charged amino acid with an uncharged or a negatively charged amino acid. In some aspects, the engineered cleavage domain comprises mutations in amino acid residues 499, 496 and 486 in addition to the mutations in one or more amino acid residues 416, 422, 447, 448, or 525.

In some aspects, the compositions described herein include engineered cleavage domains of FokI that form obligate heterodimers as described, for example, in U.S. Pat. Nos. 7,914,796; 8,034,598; 8,961,281 and 8,623,618; U.S. Patent Publication Nos. 20080131962 and 20120040398. Thus, in some aspects, the present disclosure provides fusion proteins wherein the engineered cleavage domain comprises a polypeptide in which the wild-type Gln (Q) residue at position 486 is replaced with a Glu (E) residue, the wild-type Ile (I) residue at position 499 is replaced with a Leu (L) residue and the wild-type Asn (N) residue at position 496 is replaced with an Asp (D) or a Glu (E) residue (“ELD” or “ELE”) in addition to one or more mutations at positions 416, 422, 447, 448, or 525 (numbered relative to wild-type FokI shown herein).

In some aspects, the engineered cleavage domains are derived from a wild-type FokI cleavage domain and comprise mutations in the amino acid residues 490, 538 and 537, numbered relative to wild-type FokI in addition to the one or more mutations at amino acid residues 416, 422, 447, 448, or 525. In some aspects, the present disclosure provides a fusion protein, wherein the engineered cleavage domain comprises a polypeptide in which the wild-type Glu (E) residue at position 490 is replaced with a Lys (K) residue, the wild-type Ile (I) residue at position 538 is replaced with a Lys (K) residue, and the wild-type His (H) residue at position 537 is replaced with a Lys (K) residue or an Arg (R) residue (“KKK” or “KKR”) (see U.S. Pat. No. 8,962,281, incorporated by reference herein) in addition to one or more mutations at positions 416, 422, 447, 448, or 525. See, e.g., U.S. Pat. Nos. 7,914,796; 8,034,598 and 8,623,618, the disclosures of which are incorporated by reference in its entirety for all purposes. In some aspects, the engineered cleavage domain comprises the “Sharkey” and/or “Sharkey′” mutations (see Guo et al, (2010) J. Mol. Biol. 400(1):96-107).

In some aspects, the engineered cleavage domains are derived from a wild-type FokI cleavage domain and comprise mutations in the amino acid residues 490, and 538, numbered relative to wild-type FokI or a FokI homologue in addition to the one or more mutations at amino acid residues 416, 422, 447, 448, or 525. In some aspects, the present disclosure provides a fusion protein, wherein the engineered cleavage domain comprises a polypeptide in which the wild-type Glu (E) residue at position 490 is replaced with a Lys (K) residue, and the wild-type Ile (I) residue at position 538 is replaced with a Lys (K) residue (“KK”) in addition to one or more mutations at positions 416, 422, 447, 448, or 525. In some aspects, the present disclosure provides a fusion protein, wherein the engineered cleavage domain comprises a polypeptide in which the wild-type Gln (Q) residue at position 486 is replaced with an Glu (E) residue, and the wild-type Ile (I) residue at position 499 is replaced with a Leu (L) residue (“EL”) (See U.S. Pat. No. 8,034,598, incorporated by reference herein) in addition to one or more mutations at positions 416, 422, 447, 448, or 525.

In some aspects, the present disclosure provides a fusion protein wherein the engineered cleavage domain comprises a polypeptide in which the wild-type amino acid residue at one or more of positions 387, 393, 394, 398, 400, 402, 416, 422, 427, 434, 439, 441, 447, 448, 469, 487, 495, 497, 506, 516, 525, 529, 534, 559, 569, 570, 571 in the FokI catalytic domain are mutated.

In some aspects, the one or more mutations alter the wild type amino acid from a positively charged residue to a neutral residue or a negatively charged residue. In some aspects, the mutants described can also be made in a FokI domain comprising one or more additional mutations. In some aspects, these additional mutations are in the dimerization domain, e.g. at positions 418, 432, 441, 481, 483, 486, 487, 490, 496, 499, 523, 527, 537, 538 and/or 559. Non-limiting examples of mutations include mutations (e.g., substitutions) of the wild-type residues of any cleavage domain (e.g., FokI or homologue of FokI) at positions 393, 394, 398, 416, 421, 422, 442, 444, 472, 473, 478, 480, 525 or 530 with any amino acid residue (e.g., K393X, K394X, R398X, R416S, D421X, R422X, K444X, S472X, G473X, S472, P478X, G480X, K525X, and A530X, where the first residue depicts wild-type and X refers to any amino acid that is substituted for the wild-type residue). In some aspects, X is E, D, H, A, K, S, T, D or N. Other exemplary mutations include S418E, S418D, S446D, S446R, K448A, I479Q, I479T, Q481A, Q481N, Q481E, A530E and/or A530K wherein the amino acid residues are numbered relative to full length FokI wild-type cleavage domain and homologues thereof. In some aspects, combinations can include 416 and 422, a mutation at position 416 and K448A, K448A and I479Q, K448A and Q481A and/or K448A and a mutation at position 525. In some aspects, the wild-residue at position 416 may be replaced with a Glu (E) residue (R416E), the wild-type residue at position 422 is replaced with a His (H) residue (R422H), and the wild-type residue at position 525 is replaced with an Ala (A) residue. The cleavage domains as described herein can further include additional mutations, including but not limited to at positions 432, 441, 483, 486, 487, 490, 496, 499, 527, 537, 538 and/or 559, for example dimerization domain mutants (e.g., ELD, KKR) and or nickase mutants (mutations to the catalytic domain).

In some aspects, the nFokELD protein is fused to a zinc finger DNA binding domain that binds the nucleic acid sequence as set forth in SEQ ID NO: 9 [[GCCACCATGGAGTTG]]. In some aspects, the nFokELD protein comprises five fingers: F1 comprising SEQ ID NO: 10 [[RPYTLRL]], F2 comprising SEQ ID NO: 11 [[RSANLTR]], F3 comprising SEQ ID NO: 12 [[RSDALST]], F4 comprising SEQ ID NO: 13 [[DRSTRTK]], and F5 comprising SEQ ID NO: 14 [[DRSTRTK]]. In some aspects, the cFokKKR protein is fused to a zinc finger DNA binding domain that binds the nucleic acid sequence as set forth in SEQ ID ON: 15 [[CTAGAAGGTGGCTACCTG]]. In some aspects, the cFokKKR protein is fused to a zinc finger DNA binding domain that comprising six fingers: F1 comprising SEQ ID NO: 16 [[RSDVLSA]], F2 comprising SEQ ID NO: 17 [[DRSNRIK]], F3 comprising SEQ ID NO: 18 [[DRSHLTR]], F4 comprising SEQ ID NO: 19 [[LKQHLTR]], F5 comprising SEQ ID NO: 20 [[QSGNLAR]], and F6 comprising SEQ ID NO: 21 [[QSTPRTT]].

In some aspects, a zinc finger nuclease pair comprises (1) a first ZFN comprising (a) a first DNA binding domain, which comprises five fingers: F1 comprising SEQ ID NO: 10 [[RPYTLRL]], F2 comprising SEQ ID NO: 11 [[RSANLTR]], F3 comprising SEQ ID NO: 12 [[RSDALST]], F4 comprising SEQ ID NO: 13 [[DRSTRTK]], and F5 comprising SEQ ID NO: 14 [[DRSTRTK]] and (b) a first cleavage domain comprising an nFokELD protein and (2) a second ZFN comprising (a) a second DNA binding domain, which comprises six fingers: F1 comprising SEQ ID NO: 16 [[RSDVLSA]], F2 comprising SEQ ID NO: 17 [[DRSNRIK]], F3 comprising SEQ ID NO: 18 [[DRSHLTR]], F4 comprising SEQ ID NO: 19 [[LKQHLTR]], F5 comprising SEQ ID NO: 20 [[QSGNLAR]], and F6 comprising SEQ ID NO: 21 [[QSTPRTT]] and (b) a second cleavage domain comprising a cFokKKR protein.

In some aspects, the nFokELD(Q481E) protein is fused to a zinc finger DNA binding domain that binds the nucleic acid sequence as set forth in SEQ ID NO: 38 [[ATTGCT and GAACCGTCCGGG]]. In some aspects, the nFokELD(Q481) protein comprises six fingers: F1 comprising SEQ ID NO: 23 [[RSDHLSR]], F2 comprising SEQ ID NO: 24 [[DSSDRKK]], F3 comprising SEQ ID NO: 25 [[RSDTLSE]], F4 comprising SEQ ID NO: 26 [[QSGDLTR]], and F5 comprising SEQ ID NO: 27 [[QSSDLSR]], and F6 comprising SEQ ID NO: 28 [[YKWTLRN]]. In some aspects, the cFokKKR protein is fused to a zinc finger DNA binding domain that binds the nucleic acid sequence as set forth in SEQ ID ON: 39 [[GATCCTGCAGGCCAT]]. In some aspects, the cFokKKR protein is fused to a zinc finger DNA binding domain that comprising five fingers: F1 comprising SEQ ID NO: 30 [[SNQNLTT]], F2 comprising SEQ ID NO: 31 [[DRSHLAR]], F3 comprising SEQ ID NO: 32 [[QSGDLTR]], F4 comprising SEQ ID NO: 33 [[WKHDLTN]], and F5 comprising SEQ ID NO: 34 [[TSGNLTR]].

In some aspects, nFokELD(K525S) protein is fused to a zinc finger DNA binding domain that binds the nucleic acid sequence as set forth in SEQ ID NO: 38 [[ATTGCTT and GAACCGTCCGGG]]. In some aspects, the nFokELD(K525S) protein comprises six fingers: F1 comprising SEQ ID NO: 23 [[RSDHLSR]], F2 comprising SEQ ID NO: 24 [[DSSDRKK]], F3 comprising SEQ ID NO: 25 [[RSDTLSE]], F4 comprising SEQ ID NO: 26 [[QSGDLTR]], and F5 comprising SEQ ID NO: 27 [[QSSDLSR]], and F6 comprising SEQ ID NO: 28 [[YKWTLRN]].

In some aspects, the nFokKKR(I479T) protein is fused to a zinc finger DNA binding domain that binds the nucleic acid sequence as set forth in SEQ ID ON: 39 [[GATCCTGCAGGCCAT]]. In some aspects, the nFokKKR (I479T) protein is fused to a zinc finger DNA binding domain that comprising five fingers: F1 comprising SEQ ID NO: 30 [[SNQNLTT]], F2 comprising SEQ ID NO: 31 [[DRSHLAR]], F3 comprising SEQ ID NO: 32 [[QSGDLTR]], F4 comprising SEQ ID NO: 33 [[WKHDLTN]], and F5 comprising SEQ ID NO: 34 [[TSGNLTR]].

In some aspects, a zinc finger nuclease pair comprises (1) a first ZFN comprising (a) a first DNA binding domain, which comprises six fingers: F1 comprising SEQ ID NO: 23 [[RSDHLSR]], F2 comprising SEQ ID NO: 24 [[DSSDRKK]], F3 comprising SEQ ID NO: 25 [[RSDTLSE]], F4 comprising SEQ ID NO: 26 [[QSGDLTR]], and F5 comprising SEQ ID NO: 27 [[QSSDLSR]], and F6 comprising SEQ ID NO: 28 [[YKWTLRN]] and (b) a first cleavage domain comprising an nFokELD protein and (2) a second ZFN comprising (a) a second DNA binding domain, which comprises six fingers: F1 comprising SEQ ID NO: 30 [[SNQNLTT]], F2 comprising SEQ ID NO: 31 [[DRSHLAR]], F3 comprising SEQ ID NO: 32 [[QSGDLTR]], F4 comprising SEQ ID NO:33 [[WKHDLTN]], and F5 comprising SEQ ID NO: 34 [[TSGNLTR]] and (b) a second cleavage domain comprising a nFokKKR protein.

Examples of the ZFN pairs comprising DNA binding domains and FokI protein are shown in Table 5.

| | ZFN | Binding sequence | | | | | | | |
|---|---|---|---|---|---|---|---|---|---|
| Site | ID | (capital letter) | F1 | F2 | F3 | F4 | F5 | F6 | FokI |
| B | [symbol] | gtGCCACCATGGAGTTGgg | RPYTLRL | RSANLTR | RSDALST | DRSTRTK | DRSTRTK | | [symbol] |
| | [symbol] | ccCTAGAAGGTGGCTACCTGga | ^{∧}RSDVLSA | DRSNRIW | ^{∧}DRSHLTR | LKQHLTR | ^{∧}QSGNLAR | QSTPRTT | [symbol] |
| G | [symbol] | ccATTGCTtGAACCGTCCGGGgg | RSDHLSR | DSGDRKK | RSDTLSE | QSGDLTR | QSGDLSR | YKWTLRN | [symbol] |
| | [symbol] | caGATCCTGCAGGCCATag | SNQNLTT | DRSHLAR | ^{∧}QSGDLTR | ^{∧}WKHDLTN | TSGNLTR | | [symbol] |

[symbol]

indicates data missing or illegible when filed

### IV. Polynucleotides and Vectors

The present disclosure also provides a polynucleotide encoding a ZFN of the present disclosure and/or a vector comprising the polynucleotide operably linked to a regulatory element. In some aspects, the polynucleotide comprises a polycistronic polynucleotide encoding a ZFN of the present disclosure. In some aspects, the polynucleotide is a DNA molecule, or an RNA molecule.

In some aspects, the polynucleotide and/or vector comprising the polynucleotide encodes a zinc finger nuclease polypeptide that is capable of cleaving the CIITA gene between amino acid 26 and amino acid 30, e.g., amino acid 28 and amino acid 29 corresponding to SEQ ID NO: 1. In some aspects, the zinc finger nuclease is capable of cleaving the CIITA gene between amino acid 457 and amino acid 465, e.g., amino acid 461 and amino acid 462 corresponding to SEQ ID NO: 1.

In some aspects, the polynucleotide and/or vector comprising the polynucleotide encodes a DNA binding domain polypeptide capable of binding to GCCACCATGGAGTTG (SEQ ID NO: 9). In some aspects, the polynucleotide and/or vector comprising the polynucleotide encodes the DNA binding domain that binds to GCCACCATGGAGTTG (SEQ ID NO: 9) which has five zinc fingers: finger 1 (F1) comprises or consists of SEQ ID NO: 10 [RPYTLRL], finger 2 (F2) comprises or consists of SEQ ID NO: 11 [RSANLTR], finger 3 (F3) comprises or consists of SEQ ID NO: 12 [RSDALST], finger 4 (F4) comprises or consists of SEQ ID NO: 13 [DRSTRTK], and finger 5 (F5) comprises or consists of SEQ ID NO: 14 [DRSTRTK].

In some aspects, the polynucleotide and/or vector comprising the polynucleotide encodes a DNA binding domain polypeptide capable of binding to CTAGAAGGTGGCTACCTG (SEQ ID NO: 15). In some aspects, the polynucleotide and/or vector comprising the polynucleotide encodes the DNA binding domain polypeptide that binds to CTAGAAGGTGGCTACCTG (SEQ ID NO: 15), which comprises six zinc fingers: F1 comprises or consists of SEQ ID NO: 16 [RSDVLSA], F2 comprises or consists of SEQ ID NO: 17 [DRSNRIK], F3 comprises or consists of SEQ ID NO: 18 [DRSHLTR], F4 comprises or consists of SEQ ID NO: 19 [LKQHLTR], F5 comprises or consists of SEQ ID NO: 20 [QSGNLAR], and F6 comprises or consists of SEQ ID NO: 21 [QSTPRTT].

In some aspects, the polynucleotide and/or vector comprising the polynucleotide encodes a DNA binding domain polypeptide that is capable of binding to ATTGCT and/or GAACCGTCCGGG (SEQ ID NO: 38). In some aspects, the polynucleotide and/or vector comprising the polynucleotide encodes a DNA binding domain polypeptide that binds to ATTGCT and/or GAACCGTCCGGG (SEQ ID NO: 38) has six zinc fingers: F1 comprises SEQ ID NO: 23 [RSDHLSR], F2 comprises SEQ ID NO: 24 [DSSDRKK], F3 comprises SEQ ID NO: 25 [RSDTLSE], F4 comprises 26 [QSGDLTR], and F5 comprises SEQ ID NO: 27 [QSSDLSR], and F6 comprises SEQ ID NO: 28 [YKWTLRN].

In some aspects, the polynucleotide and/or vector comprising the polynucleotide encodes a DNA binding domain polypeptide capable of binding to GATCCTGCAGGCCAT (SEQ ID NO: 29). In some aspects, the DNA binding domain that binds to GATCCTGCAGGCCAT (SEQ ID NO: 29) comprises five-fingers: F1 comprises SEQ ID NO: 30 [SNQNLTT], F2 comprises SEQ ID NO: 31 [DRSHLAR], F3 comprises SEQ ID NO: 32 [QSGDLTR], F4 comprises SEQ ID NO: 33 [WKHDLTN], and F5 comprises SEQ ID NO: 34 [TSGNLTR].

In some aspects, the polynucleotide and/or vector comprising the polynucleotide encodes a zinc finger nuclease pair which cleaves a DNA sequence in a CIITA gene. In some aspects, the polynucleotide and/or vector comprising the polynucleotide encodes the zinc finger pair which is capable of cleaving the CIITA gene between amino acid 26 and amino acid 30, e.g., amino acid 28 and amino acid 29 corresponding to SEQ ID NO: 1. In some aspects, the polynucleotide and/or vector comprising the polynucleotide encodes the zinc finger nuclease pair, which comprises a first zinc finger nuclease comprising finger 1 (F1) comprises or consists of SEQ ID NO: 10 [RPYTLRL], finger 2 (F2) comprises or consists of SEQ ID NO: 11 [RSANLTR], finger 3 (F3) comprises or consists of SEQ ID NO: 12 [RSDALST], finger 4 (F4) comprises or consists of 13 [DRSTRTK], and finger 5 (F5) comprises or consists of SEQ ID NO: 14 [DRSTRTK] and a second zinc finger nuclease comprising F1 comprises or consists of SEQ ID NO: 16 [RSDVLSA], F2 comprises or consists of SEQ ID NO: 17 [DRSNRIK], F3 comprises or consists of SEQ ID NO: 18 [DRSHLTR], F4 comprises or consists of SEQ ID NO: 19 [LKQHLTR], F5 comprises or consists of SEQ ID NO: 20 [QSGNLAR], and F6 comprises or consists of SEQ ID NO: 21 [QSTPRTT].

In some aspects, the polynucleotide and/or vector comprising the polynucleotide encodes a zinc finger nuclease pair cleaves a CIITA gene between amino acid 457 and amino acid 465, e.g., amino acid 461 and amino acid 462 corresponding to SEQ ID NO: 1. In some aspects, the polynucleotide and/or vector comprising the polynucleotide encodes a zinc finger nuclease pair which comprises a first zinc finger nuclease comprising F1 comprises SEQ ID NO: 23 [RSDHLSR], F2 comprises SEQ ID NO: 24 [DSSDRKK], F3 comprises SEQ ID NO: 25 [RSDTLSE], F4 comprises 26 [QSGDLTR], and F5 comprises SEQ ID NO: 27 [QSSDLSR], and F6 comprises SEQ ID NO: 28 [YKWTLRN] and a second zinc finger nuclease comprising F1 comprises SEQ ID NO: 30 [SNQNLTT], F2 comprises SEQ ID NO: 31 [DRSHLAR], F3 comprises SEQ ID NO: 32 [QSGDLTR], F4 comprises SEQ ID NO: 33 [WKHDLTN], and F5 comprises SEQ ID NO: 34 [TSGNLTR].

In some aspects, the vector is a transfer vector. The term “transfer vector” refers to a composition of matter which comprises an isolated nucleic acid (e.g., a polynucleotide of the present disclosure) and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

In some aspects, the vector is an expression vector. The term “expression vector” refers to a vector comprising a recombinant polynucleotide (e.g., a polypeptide of the present disclosure) comprising expression control sequences operatively linked to a nucleotide sequence to be expressed. In some embodiments, an expression vector is a polycistronic expression vector. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

In some aspects, the vector is a viral vector, a mammalian vector, or bacterial vector. In some aspects, the vector is selected from the group consisting of an adenoviral vector, a lentivirus, a Sendai virus vector, a baculoviral vector, an Epstein Barr viral vector, a papovaviral vector, a vaccinia viral vector, a herpes simplex viral vector, a hybrid vector, and an adeno associated virus (AAV) vector.

In some aspects, the adenoviral vector is a third generation adenoviral vector. ADEASY™ is by far the most popular method for creating adenoviral vector constructs. The system consists of two types of plasmids: shuttle (or transfer) vectors and adenoviral vectors. The transgene of interest is cloned into the shuttle vector, verified, and linearized with the restriction enzyme PmeI. This construct is then transformed into ADEASIER-1 cells, which are BJ5183 E. coli cells containing PADEASY™. PADEASY™ is a ˜33 Kb adenoviral plasmid containing the adenoviral genes necessary for virus production. The shuttle vector and the adenoviral plasmid have matching left and right homology arms which facilitate homologous recombination of the transgene into the adenoviral plasmid. One can also co-transform standard BJ5183 with supercoiled PADEASY™ and the shuttle vector, but this method results in a higher background of non-recombinant adenoviral plasmids. Recombinant adenoviral plasmids are then verified for size and proper restriction digest patterns to determine that the transgene has been inserted into the adenoviral plasmid, and that other patterns of recombination have not occurred. Once verified, the recombinant plasmid is linearized with PacI to create a linear dsDNA construct flanked by ITRs. 293 or 911 cells are transfected with the linearized construct, and virus can be harvested about 7-10 days later. In addition to this method, other methods for creating adenoviral vector constructs known in the art at the time the present application was filed can be used to practice the methods disclosed herein.

In other aspects, the viral vector is a retroviral vector, e.g., a lentiviral vector (e.g., a third or fourth generation lentiviral vector). The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

Lentiviral vectors are usually created in a transient transfection system in which a cell line is transfected with three separate plasmid expression systems. These include the transfer vector plasmid (portions of the HIV provirus), the packaging plasmid or construct, and a plasmid with the heterologous envelop gene (env) of a different virus. The three plasmid components of the vector are put into a packaging cell which is then inserted into the HIV shell. The virus portions of the vector contain insert sequences so that the virus cannot replicate inside the cell system. Current third generation lentiviral vectors encode only three of the nine HIV-1 proteins (Gag, Pol, Rev), which are expressed from separate plasmids to avoid recombination-mediated generation of a replication-competent virus. In fourth generation lentiviral vectors, the retroviral genome has been further reduced (see, e.g., TAKARA® LENTI-X™ fourth-generation packaging systems).

In some aspects, the present disclosure comprises a polynucleotide sequence encoding ZFN pairs 76867-2A-82862 and/or 87254-2A-84221, described herein.

In some aspects, multiple protein units of the constructs herein are expressed in a single open reading frame (ORF), thereby creating a single polypeptide having multiple protein units, wherein at least one protein is a first ZFN comprising a ZF DNA-binding domain that binds to a target site in the CIITA gene, and at least one protein is a second ZFN comprising a ZF DNA-binding domain that binds to a target site in the CIITA gene. In some aspects, an amino acid sequence or linker containing a high efficiency cleavage site is disposed between each protein expressed by the expression construct described herein. As used herein, high cleavage efficiency is defined as more than 50%, more than 70%, more than 80%, or more than 90% of the translated protein is cleaved. Cleavage efficiency can be measured by Western Blot analysis.

Non-limiting examples of high efficiency cleavage sites include porcine teschovirus-1 2A (P2A), FMDV 2A (abbreviated herein as F2A); equine rhinitis A virus (ERAV) 2A (E2A); and Thoseaasigna virus 2A (T2A), cytoplasmic polyhedrosis virus 2A (BmCPV2A) and flacherie Virus 2A (BmIFV2A), or a combination thereof. In some aspects, the high efficiency cleavage site is P2A. High efficiency cleavage sites are described in Kim et al. (2011) High Cleavage Efficiency of a 2A Peptide Derived from Porcine Teschovirus-1 in Human Cell Lines, Zebrafish and Mice. PLOS ONE 6(4): el8556, the contents of which are incorporated herein by reference.

In some aspects, a polynucleotide of the present disclosure encodes a ZFN comprising an amino acid sequence having at least about 70%, at least about 80%, at least about 85%, at least about 90%, at least about 95%, at least about 96%, at least about 97%, at least about 98%, at least about 99%, or about 100% sequence identity to SEQ ID NO: 5

(MDYKDHDGDYKDHDIDYKDDDDKMAPKKKRKVGIHGVPAAMGQLVKS
ELEEKKSELRHKLKYVPHEYIELIEIARNSTQDRILEMKVMEFFMKVY
GYRGKHLGGSRKPDGAIYTVGSPIDYGVIVDTKAYSGGYNLPIGQADE
MERYVEENQTRDKHLNPNEWWKVYPSSVTEFKFLFVSGHFKGNYKAQL
TRLNHITNCNGAVLSVEELLIGGEMIKAGTLTLEEVRRKFNNGEINFS
GAQGSTLDFRPFQCRICMRNFSRPYTLRLHIRTHTGEKPFACDICGRK
FARSANLTRHTKIHTGSQKPFQCRICMRNFSRSDALSTHIRTHTGEKP
FACDICGRKFADRSTRTKHTKIHTGEKPFQCRICMRKFADRSTRTKHT
KIHLRQKD.

In some aspects, a polynucleotide of the present disclosure encodes a ZFN comprising an amino acid sequence having at least about 70%, at least about 80%, at least about 85%, at least about 90%, at least about 95%, at least about 96%, at least about 97%, at least about 98%, at least about 99%, or about 100% sequence identity to SEQ ID NO: 6

(MDYKDHDGDYKDHDIDYKDDDDKMAPKKKRKVGIHGVPAAMAERPFQ
CRICMQNFSRSDVLSAHIRTHTGEKPFACDICGKKFADRSNRIKHTKI
HTGSQKPFQCRICMQNFSDRSHLTRHIRTHTGEKPFACDICGRKFALK
QHLTRHTKIHTGEKPFQCRICMQNFSQSGNLARHIRTHTGEKPFACDI
CGRKFAQSTPRTTHTKIHLRGSQLVKSELEEKKSELRHKLKYVPHEYI
ELIEIARNSTQDRILEMKVMEFFMKVYGYRGKHLGGSRKPDGAIYTVG
SPIDYGVIVDTKAYSGGYNLPIGQADEMQRYVKENQTRNKHINPNEWW
KVYPSSVTEFKFLFVSGHFKGNYKAQLTRLNRKTNCNGAVLSVEELLI
GGEMIKAGTLTLEEVRRKFNNGEINF).

In some aspects, a polynucleotide of the present disclosure encodes a ZFN comprising an amino acid sequence having at least about 70%, at least about 80%, at least about 85%, at least about 90%, at least about 95%, at least about 96%, at least about 97%, at least about 98%, at least about 99%, or about 100% sequence identity to SEQ ID NO: 54

(MDYKDHDGDYKDHDIDYKDDDDKMAPKKKRKVGIHGVPAAMGQLVKS
ELEEKKSELRHKLKYVPHEYIELIEIARNSTQDRILEMKVMEFFMKVY
GYRGKHLGGSRKPDGAIYTVGSPIDYGVIVDTKAYSGGYNLPIGQADE
MERYVEENQTRDKHLNPNEWWKVYPSSVTEFKFLFVSGHFSGNYKAQL
TRLNHITNCNGAVLSVEELLIGGEMIKAGTLTLEEVRRKFNNGEINFS
GTPHEVGVYTLRPFQCRICMRNFSRSDHLSRHIRTHTGEKPFACDICG
RKFADSSDRKKHTKIHTGEKPFQCRICMRNFSRSDTLSEHIRTHTGEK
PFACDICGRKFAQSGDLTRHTKIHTHPRAPIPKPFQCRICMRNFSQSS
DLSRHIRTHTGEKPFACDICGRKFAYKWTLRNHTKIHLRQKD).

In some aspects, a polynucleotide of the present disclosure encodes a ZFN comprising an amino acid sequence having at least about 70%, at least about 80%, at least about 85%, at least about 90%, at least about 95%, at least about 96%, at least about 97%, at least about 98%, at least about 99%, or about 100% sequence identity to SEQ ID NO: 56

(QLVKSELEEKKSELRHKLKYVPHEYIELIEIARNSTQDRILEMKVME
FFMKVYGYRGKHLGGSRKPDGAIYTVGSPIDYGVIVDTKAYSGGYNLP
TGQADEMQRYVKENQTRNKHINPNEWWKVYPSSVTEFKFLFVSGHFKG
NYKAQLTRLNRKTNCNGAVLSVEELLIGGEMIKAGTLTLEEVRRKFNN
GEINFSGTPHEVGVYTLRPFQCRICMRNFSSNQNLTTHIRTHTGEKPF
ACDICGRKFADRSHLARHTKIHTGEKPFQCRICMQKFAQSGDLTRHTK
IHTGEKPFQCRICMQNFSWKHDLTNHIRTHTGEKPFACDICGRKFATS
GNLTRHTKIHLRQKD).

In some aspects, a polynucleotide of the present disclosure comprises a polynucleotide sequence having at least about 70%, at least about 80%, at least about 85%, at least about 90%, at least about 95%, at least about 96%, at least about 97%, at least about 98%, at least about 99%, or about 100% sequence identity to SEQ ID NOs: 53, 55, or 57.

In some aspects, a polynucleotide of the present disclosure comprises a sequence at least about 70%, at least about 80%, at least about 90%, at least about 95%, at least about 96%, at least about 97%, or least about 98%, at least about 99% or about 100% sequence identity to SEQ ID NO 39.

In some aspects, a polynucleotide of the present disclosure encodes a ZFN comprising an amino acid sequence having at least about 70%, at least about 80%, at least about 85%, at least about 90%, at least about 95%, at least about 96%, at least about 97%, at least about 98%, at least about 99%, or about 100% sequence identity to SEQ ID NO: 7

(MDYKDHDGDYKDHDIDYKDDDDKMAPKKKRKVGIHGVPAAMGQLVKS
ELEEKKSELRHKLKYVPHEYIELIEIARNSTQDRILEMKVMEFFMKVY
GYRGKHLGGSRKPDGAIYTVGSPIDYGVIVDTKAYSGGYNLPIGQADE
MERYVEENQTRDKHLNPNEWWKVYPSSVTEFKFLFVSGHFKGNYKAQL
TRLNHITNCNGAVLSVEELLIGGEMIKAGTLTLEEVRRKFNNGEINFS
GTPHEVGVYTLRPFQCRICMRNFSRSDHLSRHIRTHTGEKPFACDICG
RKFADSSDRKKHTKIHTGEKPFQCRICMRNFSRSDTLSEHIRTHTGEK
PFACDICGRKFAQSGDLTRHTKIHTHPRAPIPKPFQCRICMRNFSQSS
DLSRHIRTHTGEKPFACDICGRKFAYKWTLRNHTKIHLRQKD).

In some aspects, a polynucleotide of the present disclosure encodes a ZFN comprising an amino acid sequence having at least about 70%, at least about 80%, at least about 85%, at least about 90%, at least about 95%, at least about 96%, at least about 97%, at least about 98%, at least about 99%, or about 100% sequence identity to SEQ ID NO: 8

(MDYKDHDGDYKDHDIDYKDDDDKMAPKKKRKVGIHGVPAAMGQLVKS
ELEEKKSELRHKLKYVPHEYIELIEIARNSTQDRILEMKVMEFFMKVY
GYRGKHLGGSRKPDGAIYTVGSPIDYGVIVDTKAYSGGYNLPIGQADE
MQRYVKENQTRNKHINPNEWWKVYPSSVTEFKFLFVSGHFKGNYKAQL
TRLNRKTNCNGAVLSVEELLIGGEMIKAGTLTLEEVRRKFNNGEINFS
GTPHEVGVYTLRPFQCRICMRNFSSNQNLTTHIRTHTGEKPFACDICG
RKFADRSHLARHTKIHTGEKPFQCRICMQKFAQSGDLTRHTKIHTGEK
PFQCRICMQNFSWKHDLTNHIRTHTGEKPFACDICGRKFATSGNLTRH
TKIHLRQKD).

In some aspects, a polynucleotide of the present disclosure comprises a sequence at least about 70%, at least about 80%, at least about 90%, at least about 95%, at least about 96%, at least about 97%, or least about 98%, at least about 99% sequence identity to SEQ ID NO: 40.

In some aspects, the vector comprises a polynucleotide a sequence at least about 70%, at least about 80%, at least about 90%, at least about 95%, at least about 96%, at least about 97%, or least about 98%, at least about 99% sequence identity to SEQ ID NO 39 or SEQ ID NO: 40 or SEQ ID NO: 57.

In some aspects, the vector comprises a polynucleotide a sequence at least about 70%, at least about 80%, at least about 90%, at least about 95%, at least about 96%, at least about 97%, or least about 98%, at least about 99% sequence identity to SEQ ID NO 39 or SEQ ID NO: 53 or SEQ ID NO: 57.

In some aspects, the vector comprises a polynucleotide a sequence at least about 70%, at least about 80%, at least about 90%, at least about 95%, at least about 96%, at least about 97%, or least about 98%, at least about 99% sequence identity to SEQ ID NO 39 or SEQ ID NO: 55.

### V. Cells

The present disclosure also provides a genetically modified cell comprising a polynucleotide construct encoding a ZFN targeting CIITA gene or a ZFN targeting CIITA gene. In some aspects, the ZFNs described herein are recombinantly expressed by a cell genetically modified to express the construct, wherein the cell comprises one or more of the polynucleotide sequences or the vectors encoding the ZFNs of the present disclosure. In some aspects, the cell is a genetically engineered cell by the ZFN targeting CIITA gene or a polynucleotide construct encoding the ZFN, wherein the cell expresses a reduced level of a CIITA protein or no expression of a CIITA gene.

In some aspects, the genetically modified cell disclosed herein has been transfected with a polynucleotide or vector encoding the protein components (e.g., ZFNs targeting CIITA gene) of the present disclosure. The term “transfected” (or equivalent terms “transformed” and “transduced”) refers to a process by which exogenous nucleic acid, e.g., a polynucleotide or vector encoding a protein of the present disclosure, is transferred or introduced into the genome of the host cell, e.g., a T cell. A “transfected” cell is one which has been transfected, transformed or transduced with exogenous nucleic acid, e.g., a polynucleotide or vector encoding the proteins of the present disclosure. The cell includes the primary subject cell and its progeny.

In some aspects, the cell (e.g., T cell) is transfected with a vector of the present disclosure, e.g., an adeno associated virus (AAV) vector or a lentiviral vector. In some such aspects, the cell may stably express the proteins of the present disclosure.

In some aspects, the cell (e.g., T cell) is transfected with a nucleic acid, e.g., mRNA, cDNA, DNA, encoding the proteins of the present disclosure. In some such aspects, the cell may transiently express the proteins of the present disclosure. For example, an RNA construct can be directly transfected into a cell. A method for generating mRNA for use in transfection involves in vitro transcription (IVT) of a template with specially designed primers, followed by polyA addition, to produce a construct containing 3′ and 5′ untranslated sequence (UTR), a 5′ cap, the nucleic acid to be expressed, and a poly A tail, typically 50-2000 bases in length. RNA so produced can efficiently transfect different kinds of cells. In some aspects, the template includes sequences for the ZFNs of the present disclosure. In an aspect, an RNA vector is transduced into a T cell by electroporation.

In some aspects, the coding sequences for the ZFN polypeptides disclosed herein can be placed on separate expression constructs. In some aspects, the coding sequences for the ZFN polypeptides disclosed herein can be placed on a single expression construct.

In some aspects, the cell is a T cell, a NK cell, a tumor infiltrating lymphocyte, a stem cell, Mesenchymal stem cells (MSC), hematopoietic stem cells (HSC), fibroblasts, cardiomyocytes, pancreatic islet cells, or a blood cell. In some aspects, the cells are allogenic or autologous.

In some aspects, T cells can be obtained from a number of sources, including peripheral blood mononuclear cells, bone marrow, lymph node tissue, cord blood, thymus tissue, tissue from a site of infection, ascites, pleural effusion, spleen tissue, and tumors.

The source of the genetically modified cells of the present disclosure may be a patient to be treated (i.e., autologous cells) or from a donor who is not the patient to be treated (e.g., allogeneic cells). In some embodiments, the engineered immune cells are engineered T cells. The T cells herein may be CD4⁺CD8⁻ (i.e., CD4 single positive) T cells, CD4⁻CD8⁺ (i.e., CD8 single positive) T cells, or CD4⁺CD8⁺ (double positive) T cells. Functionally, the T cells may be cytotoxic T cells, helper T cells, natural killer T cells, suppressor T cells, or a mixture thereof. The T cells to be engineered may be autologous or allogeneic.

Primary immune cells, including primary T cells, can be obtained from a number of tissue sources, including peripheral blood mononuclear cells (PBMCs), bone marrow, lymph node tissue, cord blood, thymus tissue, tissue from a site of infection, ascites, pleural effusion, spleen tissue, and/or tumor tissue. Leukocytes, including PBMCs, may be isolated from other blood cells by well-known techniques, e.g., FICOLL™ separation and leukapheresis. Leukapheresis products typically contain lymphocytes (including T and B cells), monocytes, granulocytes, and other nucleated white blood cells. T cells are further isolated from other leukocytes, for example, by centrifugation through a PERCOLL™ gradient or by counterflow centrifugal elutriation. A specific subpopulation of T cells, such as CD3⁺, CD25⁺, CD28⁺, CD4⁺, CD8⁺, CD45RA⁺, GITR⁺, and CD45RO⁺ T cells, can be further isolated by positive or negative selection techniques (e.g., using fluorescence-based or magnetic-based cell sorting). For example, T cells may be isolated by incubation with any of a variety of commercially available antibody-conjugated beads, such as DYNABEADS®, CELLECTION™, DETACHABEAD™ (Thermo Fisher) or MACS® cell separation products (Miltenyi Biotec), for a time period sufficient for positive selection of the desired T cells or negative selection for removal of unwanted cells.

In some instances, autologous T cells are obtained from a cancer patient directly following cancer treatment. It has been observed that following certain cancer treatments, in particular those that impair the immune system, the quality of T cells collected shortly after treatment may have an improved ability to expand ex vivo and/or to engraft after being engineered ex vivo.

Whether prior to or after genetic modification, T cells can be activated and expanded generally using methods as described, for example, in U.S. Pat. Nos. 5,858,358; 5,883,223; 6,352,694; 6,534,055; 6,797,514; 6,867,041; 6,692,964; 6,887,466; 6,905,680; 6,905,681; 6,905,874; 7,067,318; 7,144,575; 7,172,869; 7,175,843; 7,232,566; 7,572,631; and 10,786,533. Generally, T cells may be expanded in vitro or ex vivo by contact with a surface having attached thereto an agent that stimulates a CD3/TCR complex associated signal and a ligand that stimulates a costimulatory molecule on the surface of the T cells. In particular, T cell populations may be stimulated, such as by contact with an anti-CD3 antibody or antigen-binding fragment thereof, or an anti-CD3 antibody immobilized on a surface, or by contact with a protein kinase C activator (e.g., bryostatins) in conjunction with a calcium ionophore. For co-stimulation of an accessory molecule on the surface of the T cells, a ligand that binds the accessory molecule may be used. For example, a population of T cells can be contacted with an anti-CD3 antibody and an anti-CD28 antibody under conditions appropriate for stimulating proliferation of the T cells. To stimulate proliferation of either CD4⁺ T cells or CD8⁺ T cells, an anti-CD3 antibody and an anti-CD28 antibody may be employed.

The cell culture conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells. In some embodiments, the culture conditions include addition of IL-2, IL-7 and/or IL-15.

In some embodiments, the cells to be engineered can be pluripotent or multipotent cells that are differentiated into mature T cells after engineering. These non-T cells may be allogeneic and may be, for example, human embryonic stem cells, human induced pluripotent stem cells, or hematopoietic stem or progenitor cells. For ease of description, pluripotent and multipotent cells are collectively called “progenitor cells” herein.

In some aspects, allogeneic cells are engineered to reduce graft-versus-host rejection (e.g., by knocking out the endogenous CIITA genes with the ZFNs described herein). In some aspects, allogeneic cells are T cells or NK cells expressing a chimeric antigen receptor. In some aspects, allogeneic cells are T cells or NK cells expressing a T cell receptor.

### VI. Methods

In some aspects, allogeneic cells are engineered to reduce graft-versus-host rejection (e.g., by knocking out the endogenous CIITA genes with the ZFNs described herein).

In some aspects, the present disclosure provides a method of preparing a T cell, comprising isolating a T cell, and contacting the isolated T cell with the polynucleotides disclosed herein or the ZFN polypeptides disclosed herein. In some aspects, the T cell comprises a chimeric antigen receptor T cell, a T cell receptor T cell, a Treg cell, a tumor infiltrating lymphocyte, or any combination thereof.

In some aspects, the present disclosure provides a method of treating a subject in need of a cellular therapy comprising administering the isolated cells described herein to the subject. In some aspects, the isolated cells are allogeneic or autologous.

### VII. Kits

The present disclosure also provides kits, or products of manufacture comprising (i) a ZFN of the present disclosure, one or more polynucleotides encoding the ZFNs of the present disclosure, one or more vectors encoding a ZFN of the present disclosure, or a composition comprising the polynucleotide(s) or vector(s), and optionally, (ii) instructions for use, e.g., instructions for use according to the methods disclosed herein.

In some aspects, the kit or product of manufacture comprises, e.g., a polynucleotide or vector encoding a ZFN of the present disclosure, or a composition comprising a polynucleotide, vector, in at least one container, and another or more containers with transfection reagents.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986); Crooke, Antisense drug Technology: Principles, Strategies and Applications, 2nd Ed. CRC Press (2007) and in Ausubel et al. (1989) Current Protocols in Molecular Biology (John Wiley and Sons, Baltimore, Md.).

The following examples are offered by way of illustration and not by way of limitation.

### EXAMPLES

### Example 1. Preparation of Zinc Finger Nuclease

### ZFN Design

Various ZFN architectures were employed, including “tail-to-tail” pairs (e.g., FokI catalytic domains fused to the carboxy termini of the zinc finger DNA binding domains), as well as the “head-to-tail” and “head-to-head” pairs, in which one or both ZFNs bear the nuclease domain at its amino terminus. See e.g., Paschon et al., Diversifying the structure of zinc finger nucleases for high-precision genome editing, Nature Communication, 2019, 10(1): 1133-57.

To explore the benefit of applying phosphate contact changes in the initial design stage, ZFNs conforming to these architectures with or without phosphate contact changes in three of the fingers were designed for sites throughout the targeted region of the CIITA gene. A subset of the most active pairs was chosen and underwent two additional rounds of the iterations by using alternative modules, linkers, as well as number of phosphate contact changes. The ZFNs were screened in K562 cells with ZFN RNA or plasmid DNA. The most active ZFNs chosen for further improvement are referred to as “cycle 1 ZFN leads”. In the second design cycle, as a means for enhancing specificity, cycle 1 ZFN leads were redesigned using FokI variants which have been shown to improve specificity via a reduction in the rate of catalysis (Miller, Enhancing gene editing specificity by attenuating DNA cleavage kinetics, Nature Biotechnol. 2019, 37(87): 945-52). The ZFNs were screened in T cells with ZFN RNA. These ZFNs will be referred to as ‘cycle 2 ZFNs’.

### ZFN Gene Assembly

ZFN genes were assembled by linking of DNA segments encoding requisite components using standard molecular biology methods. Initial assemblies were performed into pVAX1-based ZFN expression vectors, which contain a gene expression cassette with a CMV promoter and a bovine growth hormone (BGH) polyadenylation signal sequence.

For testing in large scale T cell studies, coding sequences for the lead ZFN reagent pairs were subcloned into STV220-pVAX-GEM2UX vectors where two ZFNs were linked with a Thosea asigna virus derived 2A self-cleaving peptide (T2A) coding sequence by a Gibson cloning method using the NEBuilder HiFi DNA Assembly kit. The T2A peptide allows expression of two ZFN proteins at approximately 1:1 ratio from a single transcript (Szymczak, Nature Biotechnol, 2004, 22(5): 589-594). Construct identities were confirmed via Sanger sequencing.

Plasmid and mRNA Preparation for Screening

For use in activity screens in K562 cells, ZFN-encoding plasmids were prepared with Qiagen QIAprep 96 Turbo Kits.

ZFN-encoding RNA was prepared via the mMESSAGE mMACHINE® T7 Ultra kit (AM1345, ThermoFisher) following the manufacturer's instructions. Depending on the ZFN encoding vectors, two alternative strategies were used to make the DNA templates for in vitro RNA synthesis. For making small amounts of ZFN-encoding mRNA from the pVAX-ZFN vectors, a 5′ T7 promoter- and 3′ polyAs (n=60)-containing DNA template was used. This was generated by PCR using N80PT (5′-GCAGAGCTCTCTGGCTAACTAGAG) (SEQ ID NO: 41) and R5A60 (TTT TTTTTTTTTTTTTTTTTTTTTTTTTTTTTTTTTTTTTTTTTTTTTTTTTTTTTTTTTCTG GCAACTAGAAGGCACAG) (SEQ ID NO: 42) primers and pVAX-ZFN vectors as DNA template.

For synthesis of mRNA at large scale, SpeI-linearized STV220-pVAX-GEM2UX vectors encoding 2A-linked ZFN pair were used. For verification purposes, this mRNA batch was tested in T cells using the OC-100 MaxCyte transfection.

### Screening-Scale Transfection

Screening of ZFN candidates in the 384 well format was performed by electroporating ZFN encoding plasmids or mRNAs into K562 cells or T cells using Amaxa HT Nucleofector System (Lonza BioSciences, Inc). Subsequent screening of ZFN candidates in a 96 well format was performed by electroporating ZFN encoding mRNAs into T cells using the BTX device (Harvard Apparatus). K562 cells (ATCC) were premixed with SF Cell Line Nucleofector Solution with supplement (Lonza) and ZFN plasmids or mRNA, and electroporated using the Amaxa HT Nucleofector device. Electroporated K562 cells were placed in a 37° C. incubator for 3 days. T cells purified from healthy donor leukopaks were thawed on day 0 and activated using stimulation with anti CD3/CD28 beads and cultured for three days. On day 3, the cells were premixed with P3 Primary Cell Nucleofector Solution with supplement (Lonza) and ZFN mRNAs, and electroporated using the Amaxa HT Nucleofector device. Electroporated T cells were placed in a 30° C. incubator for 16 hours and then transferred to a 37° C. incubator until day 7. After BTX electroporation T cells were placed in a 37° C. incubator until day 7 without a 30° C. incubation step. After incubation, the K562 and T-cells were harvested and analyzed for genome modification and, in some experiments, effects on MHC class II cell-surface expression. Modification levels at the intended sites were determined using the Illumina next generation sequencing protocol given below.

### Example 2. Identification of Zinc Finger Nuclease

### T Cell Culture and RNA Delivery for Large Scale Studies

For large scale analysis of ZFN leads, a MaxCyte GT instrument was used for mRNA delivery. Cell proliferation and viability were determined on day 7 and day 10. T cell fold of expansion was calculated from day 3 to day 10. Step-by-step protocols are provided below.

### Preparation of T Cell Culture Media

Media components were brought to room temperature: X-Vivo 15 media, HEPES, Sodium pyruvate, MEM essential vitamins, GlutaMAX, and Human AB Serum. Media was prepared in a BSC hood according to table 6 below. All other supplements were added except human AB serum and filter sterilized through a 0.22 μm-filter. Then human AB was added. Formulated media was stored at 4° C. and wrapped with aluminum foil to protect from light exposure.

TABLE 6

| | Final | | % by | | |
|---|---|---|---|---|---|
| Reagent | Conc. | | volume | Vol | |
| X-Vivo 15 Serum-free Hematopoietic Cell Media (Lonza, #04-744Q) | N/A | | 90% | 900 | ml |
| HEPES (1M) (Gibco, CAT#15630080) | 20 | mM | 2% | 20 | ml |
| GlutaMax (100X) (Gibco, CAT#35050061) | 2 | mM | 1% | 10 | ml |
| Sodium Pyruvate (100 mM) (Corning, #25-000-CI) | 1 | mM | 1% | 10 | ml |
| MEM Vitamins Solution (100X) (Gibco, CAT#11120052) | 1% | | 1% | 10 | ml |
| Human AB Serum Heat-Inactivated (Valley Biomedical, #HP1022HI) | 5% | | 5% | 50 | ml |
| ProLeukin, IL-2 (lyophilized, reconstituted at 2e6 IU/ml)* *Added | 100 | IU/ml | N/A | 50 | μl |

Fresh

Thawing and activation of CD4+/CD8+ enriched T cells—Day 0: The following steps were performed: (1) Pre-warm Static media to 37° C. (2) Before thawing the selected CD4+/CD8+ T cells, prepare 15 ml or 50 ml media tubes for the first cell wash post-thaw. Warm up 10 ml media for every 1 ml cell volume. (3) Thaw vial(s) of frozen CD4+/CD8+ T cells in a 37° C. water bath by immersing the vials just below the neck and gently agitating until no ice crystal is visibly present. (4) Transfer entire volume (˜1 mL) of cell suspension dropwise to the conical tubes containing pre-aliquoted warm media. (5) Add another 500 μL warmed media to cryovial to rinse & collect remaining cells. (6) Centrifuge cells at 400×g for 6 to 10 minutes at room temperature. (7) Remove supernatant, resuspend cells in IL-2 containing media, count cells & calculate the required media (1e6 cells/ml) & CD3/CD28 beads volume (cell to bead ratio: 1 to 3). (8) Remove CD3/CD38 CTS Dynabeads (Gibco, CAT #40203D) from the refrigerator. Take volume of beads required for activation of cells at a 1:3 cell:bead ratio. (9) Wash the CD3/CD28 Dynabeads once with 1×PBS (without calcium and magnesium), then resuspend the beads in media. (10) Depending on the number of cells being activated, use appropriate tissue culture flask. Transfer cell suspension and bead suspension to the flask and add media up to desired volume to achieve a final cell suspension of 1e6 cells/ml. See table 7 below for guidance (11) Culture cells in an incubator at 37° C., 5% CO2 till day of transfection.

TABLE 7

| Flask Size Position: Upright | Volume of cells at 1e6/mL | |
|---|---|---|
| T25 | 2-10 | mL |
| T75 | 15-30 | mL |
| T162 | 35-60 | mL |
| T225 | 65-100 | mL |

### RNA Delivery for Large Scale T Cell Studies

For MaxCyte transfection of T cells 3 days after thawing the cells the following protocol was used: (1) Pre-warm Media in a 37° C. incubator prior to starting experiment. (2) Aliquot ZFN mRNA(s) into labelled 1.5 ml Eppendorf tube(s) & keep on ice. (3) Remove flasks of activated T cells from Day0 Culture from the 37° C., 5% CO2 incubator. (4) Gently break cell clumps by pipetting up and down. Sample ˜500 μl of well re-suspended cells for counting. (5) Transfer required volume of cell suspension (3e6 cells per OC-100 transfection) to 50 ml conical tube(s). Pellet cells by centrifugation at 400×g for 6 minutes at RT. Aspirate supernatant as clean as possible without disturbing the pellet. (6) Wash cells with 45 ml Hyclone MaxCyte electroporation buffer (GE Healthcare, CAT #EPB5)—ensure that cell pellet is dispersed for an effective wash. Centrifuge at 400×g for 6 minutes. Note: Presence of serum or media proteins can negatively impact transfection efficiency. (7) Remove the tube from centrifuge, transfer to BSC hood, and aspirate supernatant as clean as possible without disturbing the pellet. (8) Resuspend cells in Hyclone MaxCyte electroporation buffer to a final concentration of 30e6 cells/mL. (9) Add 100 μl cells (3e6 cells) to the designated Eppendorf tube(s) containing the intended mRNA and gently pipette the mixture 3 times to mix. Note: Mix cells with mRNA and transfect one sample at a time. (10) Transfer cell-mRNA mixture to processing assembly (PA) OC-100. (11) Insert the cells/mRNA containing PA to the MaxCyte GT and immediately start the electroporation process by clicking on the pre-programmed protocol. (12) After the electroporation process is complete, take PA to the BSC, transfer cells from the PA using a P200 pipette to designated well. (13) Carry out step (9) to (12) as quickly as possible. Repeat the step (9) to (12) until all experimental arms underwent electroporation. (14) Transfer plate with cells to a 37° C. incubator and incubate for 20 minutes. (15) After 20 minutes of incubation at 37° C., remove plates from the incubator and take them to a BSC hood. Dilute cells 10-fold with pre-warmed T cell medium supplemented with fresh IL-2 at 100 IU/ml (900 μl for OC-100). Incubate cells in a 30° C., 5% CO₂ incubator overnight.

Cell dilution—Day 4, Day7: the following protocol was used: (1) Pre-warm T cell medium to 37° C. in incubator. (2) Approximately 18 hours post electroporation or at Day4, transfer plate(s) from 30° C. to 37° C. incubator for 5 to 6 hours recovery. (3) Then dilute cells 1:4 with T cell medium containing fresh IL-2 at 100 IU/ml in appropriate plates or flasks & incubate cells at 37° C. incubator. (4) On Day 7, perform cell count and dilute cells to 5e5 cells/ml with fresh IL-2 containing media, continue to grow cells at 37° C., 5% CO2 incubator.

Cell collection for phenotyping and genotyping, and cryopreservation: the following protocol was used: (1) On Day 10, remove cell flasks from the incubator. (2) In a BSC hood, resuspend cells by pipetting. Sample ˜100 μl of cell suspension for cell count. (3) Set aside a minimum of 1e6 cells for FACS analysis and 1e6 cells for MiSeq analysis.

### Next Generation Sequencing Assay for Quantitation of ZFN Activity

To assess ZFNs for modification levels at their intended target, QuickExtract (Lucigen) lysates (for high throughput 384 or 96 well transfections) or genomic DNA purified with the Qiagen DNeasy Blood and Tissue Kit (for large scale transfections) were subjected to amplicon sequencing using Illumina Next Generation Sequencing technology.

Oligonucleotide primer pairs for amplification of 130-200 bp fragments encompassing the ZFN target sites in the human CIITA locus were designed. These primers also contain priming sequences for primers used in the second round of PCR amplification. For primer sequences used to amplify and analyze the genome targets of the final shipped ZFN pairs, see Table 8. Products of the first round of PCR amplification were used in a second round of PCR amplification using primers designed to introduce a sample specific identifier sequence (“barcode”) and constant terminal regions required for MiSeq or NextSeq sequencing (Paschon, Nature Communication, 2019, 10(1): 1133-57). The barcoded amplicons were pooled and sequenced using the MiSeq or NextSeq platform.

TABLE 8
Primers used for NGS insertion/deletion
(indel) analysis of CIITA sites B & G

| Site | Name | Sequence |
|---|---|---|
| B | CIITA- | ACACGACGCTCTTCCGATCTNNNNGTT |
| | SAPKJBBN- | GTAGGTGTCAATTTTCTGCC |
| | F | (SEQ ID NO: 43) |
| | CIITA- | GACGTGTGCTCTTCCGATCTATCTGGT |
| | SAPKJBBN- | CATAGAAGTGGTAGA |
| | R | (SEQ ID NO: 44) |
| G | CIITA- | ACACGACGCTCTTCCGATCTNNNNTCC |
| | TILVLCTW- | CCAGTACGACTTTGTCTTC |
| | F | (SEQ ID NO: 45) |
| | CIITA- | GACGTGTGCTCTTCCGATCTTCAAGAT |
| | TILVLCTW- | GTGGCTGAAAACCTC |
| | R | (SEQ ID NO: 46) |
| | i5 | AATGATACGGCGACCACCGAGATCTAC |
| | adapter | ACNNNNNNNNACACTCTTTCCCTACAC |
| | primer | GACGCTCTT (SEQ ID NO: 47) |
| | i7 | CAAGCAGAAGACGGCATACGAGATNNN |
| | adapter | NNNNNATCACGTTGTGACTGGAGTTCA |
| | primer | GACGTGTGCTCTTCCGATCT |
| | | (SEQ ID NO: 48) |

Bold indicates sequence used to prime the second round of PCT (with the i5 and i7 adapter primers), while underline indicates sequences for locus-specific priming during the initial round of PCR.

### DNA Isolation and PCR Condition for Generating Amplicons

Genomic DNA from high throughput screens was prepared using QuickExtract DNA extract solution (Lucigen). DNA from large scale transfections was prepared using the DNeasy Blood and Tissue Kit (Qiagen; Cat No. 69509) per manufacturer's instructions.

For NGS PCR using the DNeasy Blood and Tissue Kit extracted DNA, input levels of DNA were 100 ng per PCR from purified gDNA panels at 80-120 ng/μl. In addition to the DNA, the following were added to each NGS PCR reaction: 12.5 μL Phusion Hot Start Master Mix (Thermo), 1 μL each of the first round PCR primers (at a concentration of 10 μM), and water to a 25 μL total reaction volume. NGS PCR conditions were: 98° C. denaturation for 1 min, and 35 cycles at 98° C. for 15 s, 65° C. for 30 s and 72° C. for 40 s, followed by a 10 min elongation at 72° C.

Barcode PCR was performed with 1 μL of the NGS PCR product, 12.5 μL Phusion Hot Start Master Mix, 1 μL forward barcode primer, 1 μL reverse barcode primer (both at a concentration of 10 μM) and water to a 25 μL total reaction volume.

Barcode PCR conditions were: 98° C. denaturation for 1 min, and 12 cycles at 98° C. for 15 s, 65° C. for 30 s and 72° C. for 40 s, followed by a 10 min elongation at 72° C.

### Indel Quantitation

PCR-amplified target amplicons were sequenced by paired-end sequencing using next generation sequencing. Quality filtering, sequence data processing, and indel quantitation were performed as described (Miller et al. Nature Biotechnol. 2019, 37(87): 945-52). For indel quantitation, background correction was applied without windowing.

### Off-Target Assessment

Candidate off-target sites were identified using oligonucleotide duplex capture analysis as described in Miller et al. Nature Biotechnol. 2019, 37(87): 945-52. Briefly, K562 cells (ATCC, CCL-243) were maintained in RPMI1640 with 10% fetal bovine serum and 1× penicillin-streptomycin-glutamine (Corning, cat. #30-009-CI) at 37° C. with 5% CO₂. Two-hundred thousand (2e5) cells were electroporated with various doses of CIITA ZFN-encoding mRNAs (in ng) with a fixed dose (1 μM) of four nucleotide-overhang containing 27-bp oligo-capture duplex in a 20 μl transfection mix using SF Cell Line 96-well Nucleofector™ Kit (Lonza, cat. #V4SC-2096) following the manufacturer's instructions. The oligonucleotide-capture duplex was prepared by annealing the oligonucleotides 5′-P-N*N*N N GAA GAC TTC GCT ACC ACC AGT AGA C*T*G-3′ and 5′-P-N*N*N N CAG TCT ACT GGT GGT AGC GAA GTC T*T*C-3′ where P denotes 5′ phosphorylation and an asterisk indicates a phosphorothioate linkage. GFP expressing RNA was used as a negative control. Electroporated cells were recovered in 100 μl warm RPMI media with 10% fetal bovine serum and 1X penicillin-streptomycin-glutamine and transferred to 96-well tissue culture plate with 100 μl of prewarmed media. The cells were incubated at 37° C. for 48 hours. After pipette mixing, 30% of the transfected cells were harvested by centrifugation at 200×g for 10 minutes and used for indel and oligo incorporation quantification by amplicon sequencing. The remaining cells were transferred to a 24 well plate with 400 μl fresh media for scaling up the cells. These cells were maintained for another 5 days with constant supplementation of fresh media. Cells were harvested by centrifugation and saved in a pellet form at −80° C. until the construction of oligo-capture libraries.

For indel quantitation at 48 hours post-transfection, the target loci were PCR amplified and subjected to amplicon sequencing using the protocol described above. Oligonucleotide-duplex incorporation at the target site was determined using a custom shell script as described in Miller et al. (2019).

For each assessed ZFN pair, cell samples showing near-saturating levels of on-target modifications (>75% indels) and >3% duplex incorporation were identified. Genomic DNA was purified using NucleoSpin 8 Tissue kit (Macherey-Nagel, cat. #740740) following the manufacturer's instructions. DNA was quantified using Qubit™ dsDNA HS Assay Kit (Thermo Fisher, cat. #Q32854). Four hundred ng (˜120 k genomes) of genomic DNA was used to identify the candidate off-target loci following the standard oligonucleotide duplex-capture protocol (Miller, 2019).

ZFN treated T cells from screening scale studies were harvested on day 7, while ZFN treated T cells from large scale studies were harvested on day 10. Genomic DNA was isolated and assayed for on-target indel percentages as described above. For each ZFN pair, the lowest dose sample showing >75% modification was then analyzed for indel percentage at the highest ranking candidate off-target sites identified by oligonucleotide duplex capture analysis. Indels were determined at these sites using the method described above.

### Cell Expansion Rate and Viability Measurement

Cell count and viability measurement were performed using the Nexcelom Cellometer K2 instrument with the ViaStain AOPI Staining Solution (Nexcelom, #CS2-0106-25 mL).

To determine cell number and viability, 20 μl of live cell sample and 20 μl of AOPI Staining Solution were combined and mixed. 20 μl of stained sample were then added to a Cellometer Chamber on a slide and analyzed using the Cellometer K2 instrument which provides a report for live/dead cell number, live/dead cell concentration, mean diameter, and percent viability for the sample.

The fold cell expansion from Day 3 to Day 10 was determined by dividing the total number of cells of each sample on Day 10 by 3×10⁶, the number of cells used for the electroporation.

### FACS Analysis of MHC Class II Cell Surface Expression

Staining materials used for MHC class II flow cytometry were: Fixable Viability Dye eFluor 506 (eBioscience, #65-0866-14, lot #2095423), Rat Ig2a Kappa Isotype control eFluor 506 (eBioscience, Cat #69-4321-82, Lot #2094345), and APC anti-human HLA-DR, DP, DQ (Biolegend, Cat #361714, Lot #B289409). Stained cells were acquired with an Attune flow cytometer (Invitrogen) and analyzed using FlowJo software version 10.7.1.

Approximately 1 million cells for each sample were collected in a 96 deep-well plate to be stained (unmodified T cells for isotype and unstained control, mock T cells, and ZFN treated T cells). Cells were pelleted at 500×g for 5 minutes and washed twice with FACS Buffer (0.5% BSA in DPBS). 100 μl of eBioscience Fixable Viability Dye eFluor 506 (diluted 1:1000 in PBS) was added to each sample, and incubated for 30 minutes at 4 degrees, protected from light. At the end of the incubation period, cells were washed twice with 400 μl FACS buffer to remove excess viability dye. Cells were pelleted at 500×g for 5 minutes and resuspended in 50 μl of MHC class II mAb (diluted 1:20 in FACS buffer). Antibody incubation was performed at RT for 30 minutes, protected from light. Following antibody incubation, cells were washed thrice with 500 μl FACS buffer. Pellet were resuspended in 200 ul FACA buffer for acquisition readout.

Identification of Highly Active ZFN Reagents from Cycle 1 Lead Development

In the initial cycle of lead development, 180 ZFN pairs from the initial design set were screened in K562 cells using RNA transfection. A subset of most active pairs was chosen for improvement for on-target indels via two additional stages by using alternative modules, linkers, as well as number of phosphate contact changes. These ZFNs were tested with plasmid DNA in K562 cells or RNA in T cells. One representative screening data set with fifteen of the most active ZFN pairs targeting 3 unique sites obtained by transfecting ZFN RNA in T cells is provided in Table 9. These results indicated a range of titration behaviours, with the most active pairs achieving indel levels of >70% at higher doses. In our experience, activity levels seen in high-throughput T cell screens tend to underestimate modification efficiencies that will be achieved in larger scale studies.

TABLE 9
On-target modification of the most active ZFN reagents from
development cycle 1 in a high-throughput T cell screen.

| Tar- | | | | | | | | |
|---|---|---|---|---|---|---|---|---|
| get | | | % indels | | | | | |
| exon | ZFN pair | ng/ZFN | 31.25 | 62.5 | 125 | 250 | 500 | Mock |
| 2 | 76867:82860 | | 0.0 | 16.9 | 77.0 | 81.5 | 83.5 | 0.15 |
| 2 | 84111:82860 | | 49.1 | 47.2 | 71.1 | 71.9 | 67.8 | 0.15 |
| 2 | 76867:82862 | | 42.8 | 64.6 | 73.5 | 81.1 | 78.8 | 0.15 |
| 2 | 84109:82862 | | 50.4 | 43.8 | 72.8 | 67.1 | 66.5 | 0.15 |
| 2 | 84109:84118 | | 22.0 | 40.3 | 61.1 | 70.2 | 77 | 0.15 |
| 11 | 82934:84221 | | 60.5 | 73.8 | 69.5 | 67.4 | 65.1 | 0.14 |
| 11 | 84207:84221 | | 62.2 | 70.3 | NA | 74.4 | 72.6 | 0.14 |
| 11 | 84208:84221 | | 65.3 | 66.8 | 60.9 | 56.3 | 47.0 | 0.14 |
| 11 | 84214:84221 | | 68.4 | 73.2 | 73.3 | 72.6 | 68.0 | 0.14 |
| 11 | 84214:77407 | | 61.2 | 69.4 | 66.3 | 64.7 | 64.3 | 0.14 |
| 11 | 84214:84230 | | 55.2 | 71.2 | 71.2 | NA | 72.6 | 0.14 |
| 11 | 82947:77420 | | 55.6 | 69.9 | 74.8 | 79.0 | 76.5 | 0.14 |
| 11 | 82947:84242 | | 57.7 | 73.7 | 75.6 | 76.1 | 77.5 | 0.14 |
| 11 | 84231:77420 | | NA | 76.7 | 77.9 | 79.8 | 76.9 | 0.14 |
| 11 | 84231:84242 | | 64.1 | 71.0 | 76.2 | 80.8 | 68.8 | 0.14 |

### Specificity Assessment and Improvement of Cycle 1 Lead ZFNs

From the set of candidate pairs shown in Table 9, a subset was carried forward into cycle 2 of the development process, which involved two parallel activities to gauge and improve specificity. In the first of these activities, ZFN pairs were used in oligonucleotide duplex capture analysis, with follow-up indel studies to assess activity at candidate off-target sites. For the 76867: 82862 pair (see Table 9) these studies indicated good specificity, with on-target capture counts exceeding those at any other locus by ˜10 fold. Moreover, indel analysis at 23 of the highest ranked candidate off-target loci yielded low aggregate indel levels at on-target modification levels of >80%. Design features of the right ZFN 82862 include three arginine to glutamine substitutions at fingers 1, 3, and 5 to reduce ZFP binding affinity (Table 15). The highly specific performance of this pair was confirmed in large scale T cell studies using the 2A version (see Table 10).

TABLE 10
Oligonucleotide duplex capture analysis and off-target
assessment of ZFN pair 76867-2A-82862 (Site B)

| Loci identified via oligonucleotide capture | | | % indels in large scale T-cell study of 76867- | | |
|---|---|---|---|---|---|
| analysis of 76867:82862 | | | 2A-82862 | | |
| Chromosome | Base | Capture count | Treated | Mock | p-value |
| 1 | 2 | 3 | 4 | 5 | 6 |
| Chr16 | 10895316 | 1229 | 86.38 | 0.05 | 0.00 |
| chr6 | 15362686 | 129 | 0.12 | 0.01 | ns |
| chr20 | 34290252 | 41 | 0.28 | 0.02 | 0.04 |
| chr22 | 22099420 | 38 | 0.10 | 0.04 | ns |
| chr9 | 127804052 | 29 | 0.50 | 0.03 | 0.00 |
| chr6 | 31735052 | 23 | 0.05 | 0.11 | ns |
| chr6 | 41331562 | 23 | 0.17 | 0.09 | man |
| chr9 | 136518614 | 20 | 0.09 | 0.03 | ns |
| chr4 | 27308264 | 18 | 0.18 | 0.07 | ns |
| chr1 | 151347230 | 17 | 0.21 | 0.05 | man |
| chr1 | 184610470 | 13 | 0.06 | 0.00 | ns |
| chr17 | 45425654 | 13 | 0.08 | 0.00 | ns |
| chr6 | 37130508 | 13 | 0.10 | 0.04 | ns |
| chr22 | 31087122 | 11 | 0.12 | 0.03 | ns |
| chr22 | 21368356 | 10 | 0.04 | 0.03 | ns |
| chr6 | 30067282 | 9 | 0.05 | 0.02 | ns |
| chr1 | 156866756 | 9 | 0.62 | 0.61 | ns |
| chr22 | 18626926 | 9 | 0.00 | 0.03 | ns |
| chr8 | 30776248 | 9 | 0.05 | 0.04 | ns |
| chr13 | 38197748 | 8 | 0.09 | 0.02 | ns |
| chr16 | 16648524 | 8 | 0.07 | 0.05 | ns |
| chr16 | 18710542 | 8 | 0.09 | 0.03 | ns |
| chr19 | 10319978 | 8 | 0.64 | 0.16 | 0.01 |
| chr16 | 28863402 | 8 | 0.09 | 0.09 | ns |

ns: not significant; man: possible off-target site based on manual indel analysis.

For a second pair (see the 84214:84221 pair in Table 9), capture and indel analysis also yielded good performance. Design features of the right ZFN 84221 include two arginine to glutamine substitutions at fingers 3 and 4 to reduce ZFP binding affinity (Table 15). To further reduce the off-target activity, ZFN variants were assembled bearing substitutions in the FokI domain and then screened for improved specificity vs the known off-targets. This effort identified a significantly improved variant of ZFN 84214, designated 87254, with a Q481E substitution in the FokI domain (Table 15). Replacement of the 84214 ZFN with 87254 yielded a dramatic reduction in background-subtracted off-target indels (compare columns 4 and 5 of Table 11). The specificity of this pair in its 2A configuration was confirmed in large scale T cell studies (see Table 11).

TABLE 11
Oligonucleotide duplex capture analysis and off-target
assessment of ZFN pair 84214-2A-84221 (site G)

| Loci identified via capture | | | % indels in small scale | | | % indels in large-scale T- | | |
|---|---|---|---|---|---|---|---|---|
| analysis of 84214:84221 | | | followup | | | cell study of 87254-2A- 84221 | | |
| | | Capture | 84214 | 82754 | | | | p- |
| Chromosome | Base | count | 84221 | 84221 | Mock | Treated | Mock | value |
| 1 | 2 | 3 | 4 | 5 | 6 | 7 | 8 | 9 |
| chr16 | 10906874 | 1066 | 86.48 | 87.63 | 0.05 | 90.11 | 0.03 | 0.00 |
| chr11 | 66336084 | 201 | 9.43 | 0.14 | 0.01 | 0.00 | 0.00 | ns |
| chr1 | 11997242 | 173 | 0.88 | 0.04 | 0.04 | 0.08 | 0.09 | ns |
| chr19 | 17292912 | 134 | 1.06 | 0.04 | 0.06 | 0.06 | 0.04 | ns |
| chr2 | 71331736 | 101 | 3.29 | 0.27 | 0.05 | 0.21 | 0.05 | 0.02 |
| chr11 | 67071954 | 99 | 0.27 | 0.08 | 0.01 | 0.23 | 0.12 | ns |
| chr17 | 82088678 | 87 | 0.87 | 0.05 | 0.03 | 0.10 | 0.06 | ns |
| chr1 | 201703736 | 75 | 0.02 | 0.03 | 0.06 | 0.06 | 0.06 | ns |
| chr8 | 38924504 | 70 | 0.14 | 0.03 | 0.04 | 0.04 | 0.08 | ns |
| chr17 | 79993910 | 69 | 0.20 | 0.04 | 0.02 | 0.02 | 0.07 | ns |
| chr8 | 143514974 | 65 | 1.04 | 0.05 | 0.04 | 0.05 | 0.02 | ns |
| chr8 | 143948572 | 62 | 0.21 | 0.04 | 0.06 | 0.12 | 0.11 | ns |
| chr17 | 28880540 | 53 | 0.04 | 0.04 | 0.03 | 0.08 | 0.06 | ns |
| chr1 | 8318128 | 49 | 0.14 | 0.02 | 0.04 | 0.00 | 0.04 | ns |
| chr6 | 2988928 | 46 | 0.06 | 0.06 | 0.09 | 0.16 | 0.02 | ns |
| chr9 | 137548112 | 42 | 0.04 | 0.04 | 0.03 | 0.04 | 0.07 | ns |
| chr14 | 35535364 | 39 | 0.20 | 0.01 | 0.03 | ND | ND | ND |
| chr22 | 21607730 | 38 | 0.06 | 0.03 | 0.05 | 0.10 | 0.02 | ns |
| chr20 | 29878422 | 36 | 0.09 | 0.14 | 0.14 | 0.00 | 0.00 | ns |
| chr19 | 4636256 | 30 | 0.13 | 0.05 | 0.04 | 0.01 | 0.09 | ns |
| chr8 | 86514620 | 30 | 0.40 | 0.05 | 0.07 | 0.09 | 0.02 | ns |
| chr11 | 65888676 | 28 | 0.32 | 0.27 | 0.05 | 0.15 | 0.04 | ns |
| chr17 | 42313148 | 28 | 0.04 | 0.04 | 0.03 | 0.04 | 0.02 | ns |
| chr9 | 133741862 | 27 | 0.05 | 0.04 | 0.04 | 0.05 | 0.05 | ns |

ns: not significant; ND: not analyzed due to technical limitations.

The ZFN pairs resulting from these efforts-76867:82862 and 87254:84221-exhibited a high degree of cleavage specificity with aggregate off-target indel levels of <15% with on-target modification levels of >75% in large scale T cell studies (see Tables 10 and 11).

### Example 3. Large Scale Studies

Prior to large scale T cell studies, the two ZFN-encoding genes for each lead pair, 76867: 82862 (site B) and 87254: 84221 (site G), were joined via a DNA segment encoding a 2A-peptide, and the resulting fusion genes were subcloned into a STV220-pVAX-GEM2UX expression vector. The 2A peptide enables efficient production of both ZFN proteins from a single RNA transcript (Szymczak, 2004). RNAs generated from the resulting constructs were then transfected into T cells using the OC-100 MaxCyte protocol at concentrations of 50, 100, 150 and 200 μg/ml. On-target indel percentages measured by next generation sequencing at day 10 (i.e. 7 days post transfection) showed high indel levels at the lowest dose and peak indel levels at ˜96%, see Table 12. As a control, 90 μg/ml of the ZFN pair pST-TRACmR (CD19 targeting), was included in the transfection, and a modification percentage of 91.2% was obtained.

TABLE 12
On-target modification percentage of the CIITA
locus in the large scale T cell transfection

| | ZFN mRNA concentration (μg/ml) | | | | |
|---|---|---|---|---|---|
| ZFN Pair | 50 | 100 | 150 | 200 | Mock |
| 76867-2A- | 86.2 | 94.1 | 96.8 | 96.5 | 0.2 |
| 82862 (Site B) | | | | | |
| 87254-2A- | 90.5 | 95.3 | 96.9 | 97.3 | 0.3 |
| 84221 (Site G) | | | | | |

### Assessment of Aggregate Off-Target Indel Levels

A key performance requirement of the CIITA targeting ZFNs is that they exhibit high specificity, in particular aggregate off-target indel levels of <15%. In order to assess performance relative to this metric, the low-dose samples for the 76867-2A-82862 and the 87254-2A-84221 pair were characterized for % indels at the highest-ranked candidate off-target sites as determined by the oligonucleotide duplex capture studies. The results of these analyses, which are summarized in Tables 10 and 11 show activity and specificity performance which is well within the tolerances defined for this program. For the 76867-2A-82862 pair (site B), analysis of 23 candidate off-targets identified three site exhibiting statistically significant indel signal, at a background-subtracted level of 0.28%, 0.50%, 0.64%, respectively. Two other sites did not show statistically significant indel levels but were considered potential off-target sites based on manual indel analysis. For the 87254-2A-84221 pair (site G), only one off-target site exhibited statistically significant indel levels of 0.21% and no further sites showed ZFN induced indels in the manual analysis.

### Assessment of Cell Viability and Cell Expansion

To assess the effect of ZFN expression on T cell health in the large scale transfection, the cell viability was determined on day 7 and day 10 (4 and 7 days after transfection). As shown in Table 13, no large loss of cell viability was observed as compared to the mock control. Measurement of T cell expansion from day 3 through day 10 (Table 14) showed a larger than usual variation in the expansion of the mock and TRAC ZFN controls but no clear reduction of expansion at the highest CIITA ZFN mRNA input levels.

TABLE 13
Viability of CIITA ZFN transfected T cells at day 7 and day 10

| | RNA Concentration | D 7 viability | D 10 viability |
|---|---|---|---|
| ZFN Pair | (μg/ml) | (%) | (%) |
| Mock | 0 | 91.1 | 85.6 |
| 76867-2A-82862 | 50 | 89 | 88.7 |
| (Site B) | 100 | 89.7 | 91.3 |
| | 150 | 88.1 | 89.3 |
| | 200 | 87.8 | 86.2 |
| 87254-2A-84221 | 50 | 88.4 | 86 |
| (Site G) | 100 | 86.8 | 89.8 |
| | 150 | 91.1 | 90.2 |
| | 200 | 90.9 | 88.9 |
| ST-TRACmR | 90 | 92 | 93.5 |
| Control | | | |

TABLE 14
Day 3 to Day 10 expansion of CIITA transfected cells

| | RNA Concentration | Fold Expansion Day |
|---|---|---|
| ZFN Pair | (μg/ml) | 3-10 |
| Mock | 0 | 9.1 |
| 76867-2A-82862 | 50 | 18.5 |
| (Site B) | 100 | 14.7 |
| | 150 | 15.1 |
| | 200 | 14.0 |
| 87254-2A-84221 | 50 | 16.2 |
| (Site G) | 100 | 18.1 |
| | 150 | 16.7 |
| | 200 | 17.0 |
| ST-TRACmR | 90 | 29.2 |
| Control | | |

### FACS Analysis of MHC Class II Cell Surface Expression

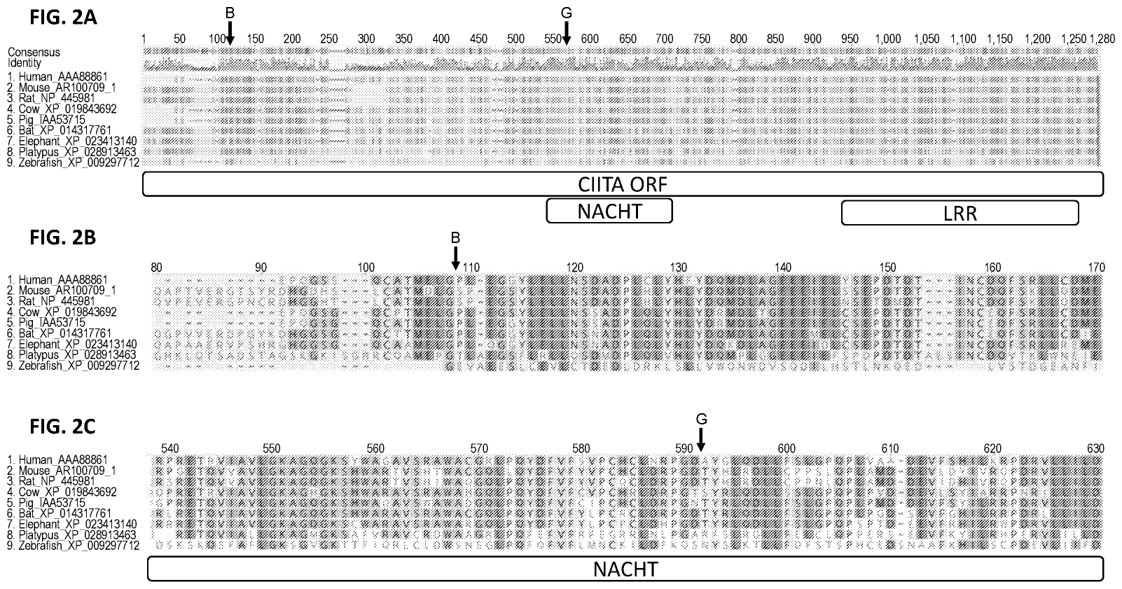

Since the ZFN mediated knock-out of CIITA function should lead to a reduction of the percentage of cells exhibiting cell surface expression of MHC class II, we performed FACS analysis on the T cells at the time of harvest, 7 days after transfection (‘day 10’). A ZFN concentration dependent reduction in MHC class II signal in the CIITA ZFN treated samples compared to the mock and TRAC ZFN control samples was observed (FIG. 2) With MHC class II reduction over 75% CIITA ZFN pair 87534-2A-84221 (site G) showed a markedly higher reduction of MHC class II levels than ZFN pair 76867-2A-82862 (site B) which reduced MHC class II levels by approximately 50%, although both ZFN pairs lead to similar and very high indel levels.

### Bioinformatics-Based Consideration of Functional Effects of Indels Sites at the ZFN Cleaving Sites

The full-length protein sequences of CIITA homologs from 9 species were aligned as shown in FIG. 2A. The regions with the cutting sites for ZFN pairs 76867:82862 (site B) and 87254:84221 (site G) are zoomed in and shown in FIGS. 2B and 2C, respectively. While both ZFN pairs cleave DNA within conserved regions, suggesting that the indels generated by the ZFNs should mediate functional disruption, the MHC class II protein knock-down levels measured by FACS demonstrated higher efficiency at the G site than B site. As shown in FIG. 2, the G site is located inside the NACHT domain. The NACHT domain is an evolutionarily conserved predicted nucleoside-triphosphatase (NTPase) domain (Koonin, Trends in Biochemical Sciences, 2000, 25 (5): 223.). Its name comes from some proteins that contain it: NAIP (NLP family apoptosis inhibitor protein), CIITA (that is, C2TA or MHC class II transcription activator), HET-E (incompatibility locus protein from Podospora anserina), and TEP1 (telomerase-associated protein). Considering the important function of the NACHT domain and the likely Rossman fold of its nucleotide binding domain, it is possible that amino acid residue changes due to in-frame indels generated at the G site are more detrimental to CIITA protein function than amino acid residue changes due to in-frame indels generated at the B site and therefore exhibit more profound MHC class II reduction.

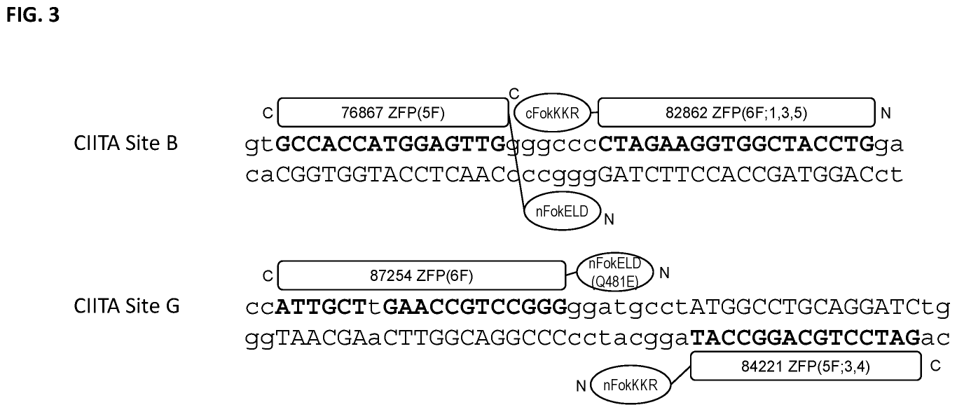

The ZFN pairs, 76867:82862 (site B) and 87254:84221 (site G), target in CIITA exons 2 and 11, respectively. Their target sites relative to the genome and to the protein sequences are shown in FIG. 1. Design information, architectures, and DNA binding sequences for the two ZFN reagents are shown in Table 15, and FIG. 3. Specifically, Table 15 below lists 6 exemplary engineered ZFNs of the present disclosure. For each ZFN, the genomic target sequence (Binding Sequence) and the DNA-binding recognition helix sequences (i.e., F1-F6) of each zinc finger within the ZFN domain are shown in a single row. “\hat{ }” in below table indicates that the arginine (R) residue at the 4th position upstream of the 1st amino acid in the indicated helix is changed to glutamine (Q). Sequence listing numbers (SEQ ID NO: #) for the sequences are shown in parentheses.

TABLE 15
CIITA ZFN DNA binding and helix sequences

| ZFN | Binding | | | | | | | |
|---|---|---|---|---|---|---|---|---|
| ID | Sequence | F1 | F2 | F3 | F4 | F5 | F6 | FokI |
| 76867 | GCCACCA | RPYTLRL | RSANLT | RSDALST | DRSTRTK | DRSTRTK | | nFokELD |
| (site | TGGAGTT | (SEQ ID | R | (SEQ ID | (SEQ ID | (SEQ ID | | |
| B) | G (SEQ | NO: 10) | (SEQ | NO: 12) | NO: 13) | NO: 14) | | |
| | ID NO: | | ID NO: | | | | | |
| | 9) | | 11) | | | | | |
| 82862 | CTAGAAG | ^{∧}DRSTRT | DRSNRI | ^{∧}DRSHLT | LKQHLTR | ^{∧}QSGNLA | QSTPR | cFokKKR |
| (site | GTGGCTA | K | K | R | (SEQ ID | R | TT | |
| B) | CCTG | (SEQ ID | (SEQ | (SEQ ID | NO: 19) | (SEQ ID | (SEQ | |
| | (SEQ ID | NO: 16) | ID NO: | NO: 18) | | NO: 20) | ID | |
| | NO: 15) | | 17) | | | | NO: | |
| | | | | | | | 21) | |
| 87254 | ATTGCTt | RSDHLSR | DSSDRK | RSDTLSE | QSGDLTR | QSSDLSR | YKWTL | nFokELD |
| (site | GAACCGT | (SEQ ID | K | (SEQ ID | (SEQ ID | (SEQ ID | RN | (Q481E) |
| G) | CCGGG | NO: 23) | (SEQ | NO: 25) | NO: 26) | NO: 27) | (SEQ | |
| | (SEQ ID | | ID NO: | | | | ID | |
| | NO: 38) | | 24) | | | | NO: | |
| | | | | | | | 28) | |
| 84221 | GATCCTG | SNQNLTT | DRSHLA | ^{∧}QSGDLT | ^{∧}WKHDLT | TSGNLTR | | nFokKKR |
| (site | CAGGCCA | (SEQ ID | R | R | N | (SEQ ID | | |
| G) | T | NO: 30) | (SEQ | (SEQ ID | (SEQ ID | NO: 34) | | |
| | (SEQ ID | | ID NO: | NO: 32) | NO: 33) | | | |
| | NO: 29) | | 31) | | | | | |
| 87278 | ATTGCTt | RSDHLSR | DSSDRK | RSDTLSE | QSGDLTR | QSSDLSR | YKWTL | nFokELD |
| (site | GAACCGT | (SEQ ID | K | (SEQ ID | (SEQ ID | (SEQ ID | RN | (K525S) |
| G) | CCGGG | NO: 23) | (SEQ | NO: 25) | NO: 26) | NO: 27) | (SEQ | |
| | (SEQ ID | | ID NO: | | | | ID | |
| | NO: 38) | | 24) | | | | NO: | |
| | | | | | | | 28) | |
| 87232 | GATCCTG | SNQNLTT | DRSHLA | ^{∧}QSGDLT | ^{∧}WKHDLT | TSGNLTR | | nFokKKR |
| (site | CAGGCCA | (SEQ ID | R | R | N | (SEQ ID | | (I479T) |
| G) | T | NO: 30) | (SEQ | (SEQ ID | (SEQ ID | NO: 34) | | |
| | (SEQ ID | | ID NO: | NO: 32) | NO: 33) | | | |
| | NO: 29) | | 31) | | | | | |

^{∧}The arginine residue at the 4th position upstream of the 1st amino acid in the indicated helix is changed to glutamine.

The ZFN pairs, 76867:82862 (site B) targets in CHITA exon 2, the ZFN pairs 87254:84221 and 87278: 87232 (site G) target in CIITA exon11. Their target sites relative to the genome and to the protein sequences are shown in FIG. 1. Design information, architectures, and DNA binding sequences for the two ZFN reagents are shown in Table 15, and FIG. 3.

TABLE 16
Complete nucleotide sequence of Target Site B
Vector I plasmid (SEQ ID NO: 39)

GCTGCTTCGCGATGTACGGGCCAGATATACGCGCATGTTCTTTCCTGCGTTATCCCCTGATTCTGTGGATAACCGT
ATTACCGCCTTTGAGTGAGCTGATACCGCTCGCCGCAGCCGAACGACCGAGCGCAGCGAGTCAGTGAGCGAGGAAG
CGGAAGAGCGCCCAATACGCAAACCGCCTCTCCCCGCGCGTTGGCCGATTCATTAATGCAGCTGGCACGACAGGTT
TCCCGACTGGAAAGCGGGCAGTGAGCGCAACGCAATTAATGTGAGTTAGCTCACTCATTAGGCACCCCAGGCTTTA
CACTTTATGCTTCCGGCTCGTATGTTGTGTGGAATTGTGAGCGGATAACAATTTCACACAGGAAACAGCTATGACC
ATGATTACGCCAAGCTCTAATACGACTCACTATAGGGAGACAAGCTTGAATACAAGCTTGCTTGTTCTTTTTGCAG
AAGCTCAGAATAAACGCTCAACTTTGGCAGATCGAATTCGCCATGGACTACAAAGACCATGACGGTGATTATAAAG
ATCATGACATCGATTACAAGGATGACGATGACAAGATGGCCCCCAAGAAGAAGAGGAAGGTCGGCATCCACGGGGT
ACCCGCCGCTATGGGACAGCTGGTGAAGAGCGAGCTGGAGGAGAAGAAGTCCGAGCTGCGGCACAAGCTGAAGTAC
GTGCCCCACGAGTACATCGAGCTGATCGAGATCGCCAGGAACAGCACCCAGGACCGCATCCTGGAGATGAAGGTGA
TGGAGTTCTTCATGAAGGTGTACGGCTACAGGGGAAAGCACCTGGGCGGAAGCAGAAAGCCTGACGGCGCCATCTA
TACAGTGGGCAGCCCCATCGATTACGGCGTGATCGTGGACACAAAGGCCTACAGCGGCGGCTACAATCTGCCTATC
GGCCAGGCCGACGAGATGGAGAGATACGTGGAGGAGAACCAGACCCGGGATAAGCACCTCAACCCCAACGAGTGGT
GGAAGGTGTACCCTAGCAGCGTGACCGAGTTCAAGTTCCTGTTCGTGAGCGGCCACTTCAAGGGCAACTACAAGGC
CCAGCTGACCAGGCTGAACCACATCACCAACTGCAATGGCGCCGTGCTGAGCGTGGAGGAGCTGCTGATCGGCGGC
GAGATGATCAAAGCCGGCACCCTGACACTGGAGGAGGTGCGGCGCAAGTTCAACAACGGCGAGATCAACTTCAGCG
GCGCTCAGGGATCTACCCTGGACTTTAGGCCCTTCCAGTGTCGAATCTGCATGCGTAACTTCAGTCGCCCGTACAC
CCTGCGCCTGCACATCCGCACCCACACCGGCGAGAAGCCTTTTGCCTGTGACATTTGTGGGAGGAAATTTGCCCGC
TCCGCCAACCTGACCCGCCATACCAAGATACACACGGGCAGCCAAAAGCCCTTCCAGTGTCGAATCTGCATGCGTA
ACTTCAGTCGTAGTGACGCCCTGAGCACGCACATCCGCACCCACACAGGCGAGAAGCCTTTTGCCTGTGACATTTG
TGGGAGGAAATTTGCCGACAGGAGCACCCGCACAAAGCATACCAAGATACACACGGGCGAGAAGCCCTTCCAGTGT
CGAATCTGCATGCGTAAGTTTGCCGACCGCTCCACCCGCACCAAGCATACCAAGATACACCTGCGGCAGAAGGACA
GATCTGGCGGCGGAGAGGGCAGAGGAAGTCTTCTAACCTGCGGTGACGTGGAGGAGAATCCCGGCCCTAGGACCAT
GGACTACAAAGACCATGACGGTGATTATAAAGATCATGACATCGATTACAAGGATGACGATGACAAGATGGCCCCC
AAGAAGAAGAGGAAGGTCGGCATTCATGGGGTACCCGCCGCTATGGCTGAGAGGCCCTTCCAGTGTCGAATCTGCA
TGCAGAACTTCAGTCGTAGTGACGTCCTGAGCGCACACATCCGCACCCACACAGGCGAGAAGCCTTTTGCCTGTGA
CATTTGTGGGAAGAAATTTGCCGACAGGAGCAACCGCATAAAGCATACCAAGATACACACGGGCAGCCAAAAGCCC
TTCCAGTGTCGAATCTGCATGCAGAACTTCAGTGACCGCTCCCACCTGACCCGCCACATCCGCACCCACACCGGCG
AGAAGCCTTTTGCCTGTGACATTTGTGGGAGGAAATTTGCCCTGAAGCAGCACCTGACCCGCCATACCAAGATACA
CACGGGCGAGAAGCCCTTCCAGTGTCGAATCTGCATGCAGAACTTCAGTCAGTCCGGCAACCTGGCCCGCCACATC
CGCACCCACACCGGCGAGAAGCCTTTTGCCTGTGACATTTGTGGGAGGAAATTTGCCCAGTCCACCCCGCGCACCA
CCCATACCAAGATACACCTGCGGGGATCCCAGCTGGTGAAGAGCGAGCTGGAGGAGAAGAAGTCCGAGCTGCGGCA
CAAGCTGAAGTACGTGCCCCACGAGTACATCGAGCTGATCGAGATCGCCAGGAACAGCACCCAGGACCGCATCCTG
GAGATGAAGGTGATGGAGTTCTTCATGAAGGTGTACGGCTACAGGGGAAAGCACCTGGGCGGAAGCAGAAAGCCTG
ACGGCGCCATCTATACAGTGGGCAGCCCCATCGATTACGGCGTGATCGTGGACACAAAGGCCTACAGCGGCGGCTA
CAATCTGCCTATCGGCCAGGCCGACGAGATGCAGAGATACGTGAAGGAGAACCAGACCCGGAATAAGCACATCAAC
CCCAACGAGTGGTGGAAGGTGTACCCTAGCAGCGTGACCGAGTTCAAGTTCCTGTTCGTGAGCGGCCACTTCAAGG
GCAACTACAAGGCCCAGCTGACCAGGCTGAACCGCAAAACCAACTGCAATGGCGCCGTGCTGAGCGTGGAGGAGCT
GCTGATCGGCGGCGAGATGATCAAAGCCGGCACCCTGACACTGGAGGAGGTGCGGCGCAAGTTCAACAACGGCGAG
ATCAACTTCTGATAACTCGAGTCTAGAAGCTCGCTTTCTTGCTGTCCAATTTCTATTAAAGGTTCCTTTGTTCCCT
AAGTCCAACTACTAAACTGGGGGATATTATGAAGGGCCTTGAGCATCTGGATTCTGCCTAATAAAAAACATTTATT
TTCATTGCTGCGCTAGAAGCTCGCTTTCTTGCTGTCCAATTTCTATTAAAGGTTCCTTTGTTCCCTAAGTCCAACT
ACTAAACTGGGGGATATTATGAAGGGCCTTGAGCATCTGGATTCTGCCTAATAAAAAACATTTATTTTCATTGCTG
CGGGACATTCTTAATTAAAAAAAAAAAAAAAAAAAAAAAAAAAAAAAAAAAAAAAAAAAAAAAAAAAAAAAAAAAA
CTAGTGGCGCCTGATGCGGTATTTTCTCCTTACGCATCTGTGCGGTATTTCACACCGCATAATCCAGCACAGTGGC
GGCCCGTTTAAACCCGCTGATCAGCCTCGACTGTGCCTTCTAGTTGCCAGCCATCTGTTGTTTGCCCCTCCCCCGT
GCCTTCCTTGACCCTGGAAGGTGCCACTCCCACTGTCCTTTCCTAATAAAATGAGGAAATTGCATCGCATTGTCTG
AGTAGGTGTCATTCTATTCTGGGGGGTGGGGTGGGGCAGGACAGCAAGGGGGAGGATTGGGAAGACAATAGCAGGC
ATGCTGGGGATGCGGTGGGCTCTATGGCTTCTACTGGGCGGTTTTATGGACAGCAAGCGAACCGGAATTGCCAGCT
GGGGCGCCCTCTGGTAAGGTTGGGAAGCCCTGCAAAGTAAACTGGATGGCTTTCTTGCCGCCAAGGATCTGATGGC
GCAGGGGATCAAGCTCTGATCAAGAGACAGGATGAGGATCGTTTCGCATGATTGAACAAGATGGATTGCACGCAGG
TTCTCCGGCCGCTTGGGTGGAGAGGCTATTCGGCTATGACTGGGCACAACAGACAATCGGCTGCTCTGATGCCGCC
GTGTTCCGGCTGTCAGCGCAGGGGCGCCCGGTTCTTTTTGTCAAGACCGACCTGTCCGGTGCCCTGAATGAACTGC
AAGACGAGGCAGCGCGGCTATCGTGGCTGGCCACGACGGGCGTTCCTTGCGCAGCTGTGCTCGACGTTGTCACTGA
AGCGGGAAGGGACTGGCTGCTATTGGGCGAAGTGCCGGGGCAGGATCTCCTGTCATCTCACCTTGCTCCTGCCGAG
AAAGTATCCATCATGGCTGATGCAATGCGGCGGCTGCATACGCTTGATCCGGCTACCTGCCCATTCGACCACCAAG
CGAAACATCGCATCGAGCGAGCACGTACTCGGATGGAAGCCGGTCTTGTCGATCAGGATGATCTGGACGAAGAGCA
TCAGGGGCTCGCGCCAGCCGAACTGTTCGCCAGGCTCAAGGCGAGCATGCCCGACGGCGAGGATCTCGTCGTGACC
CATGGCGATGCCTGCTTGCCGAATATCATGGTGGAAAATGGCCGCTTTTCTGGATTCATCGACTGTGGCCGGCTGG
GTGTGGCGGACCGCTATCAGGACATAGCGTTGGCTACCCGTGATATTGCTGAAGAGCTTGGCGGCGAATGGGCTGA
CCGCTTCCTCGTGCTTTACGGTATCGCCGCTCCCGATTCGCAGCGCATCGCCTTCTATCGCCTTCTTGACGAGTTC
TTCTGAATTATTAACGCTTACAATTTCCTGATGCGGTATTTTCTCCTTACGCATCTGTGCGGTATTTCACACCGCA
TCAGGTGGCACTTTTCGGGGAAATGTGCGCGGAACCCCTATTTGTTTATTTTTCTAAATACATTCAAATATGTATC
CGCTCATGAGACAATAACCCTGATAAATGCTTCAATAATAGCACGTGCTAAAACTTCATTTTTAATTTAAAAGGAT
CTAGGTGAAGATCCTTTTTGATAATCTCATGACCAAAATCCCTTAACGTGAGTTTTCGTTCCACTGAGCGTCAGAC
CCCGTAGAAAAGATCAAAGGATCTTCTTGAGATCCTTTTTTTCTGCGCGTAATCTGCTGCTTGCAAACAAAAAAAC
CACCGCTACCAGCGGTGGTTTGTTTGCCGGATCAAGAGCTACCAACTCTTTTTCCGAAGGTAACTGGCTTCAGCAG
AGCGCAGATACCAAATACTGTTCTTCTAGTGTAGCCGTAGTTAGGCCACCACTTCAAGAACTCTGTAGCACCGCCT
ACATACCTCGCTCTGCTAATCCTGTTACCAGTGGCTGCTGCCAGTGGCGATAAGTCGTGTCTTACCGGGTTGGACT
CAAGACGATAGTTACCGGATAAGGCGCAGCGGTCGGGCTGAACGGGGGGTTCGTGCACACAGCCCAGCTTGGAGCG
AACGACCTACACCGAACTGAGATACCTACAGCGTGAGCTATGAGAAAGCGCCACGCTTCCCGAAGGGAGAAAGGCG
GACAGGTATCCGGTAAGCGGCAGGGTCGGAACAGGAGAGCGCACGAGGGAGCTTCCAGGGGGAAACGCCTGGTATC
TTTATAGTCCTGTCGGGTTTCGCCACCTCTGACTTGAGCGTCGATTTTTGTGATGCTCGTCAGGGGGGCGGAGCCT
ATGGAAAAACGCCAGCAACGCGGCCTTTTTACGGTTCCTGGCCTTTTGCTGGCCTTTTGCTCACATGTTCTT

TABLE 17
Features of the CIITA ZFN plasmid Target Site B Vector I

| Nucleotide Position | Region/Open Reading Fram |
|---|---|
| 1-33 | Linker sequence |
| 34-397 | Linker sequence |
| 398-417 | T7 promoter/primer site |
| 415 | T7 transcription start site |
| 418-438 | Linker sequence |
| 439-489 | 5′UTR element |
| 490-498 | Linker sequence |
| 499-501 | Translation start codon |
| 502-567 | Hydrophilic peptide |
| 568-573 | Linker sequence |
| 574-594 | Nuclear localization signal |
| 595-624 | Linker sequence |
| 625-1212 | FokI ELD nuclease catalytic domain |
| 1213-1242 | FokI-ZFP N6a linker |
| 1243-1671 | Zinc finger 76867 DNA binding domain |
| 1672-1777 | Linker sequence |
| 1778-1740 | 2A self-cleaving peptide |
| 1741-1749 | Linker sequence |
| 1750-1815 | Hydrophilic peptide |
| 1816-18216 | Linker sequence |
| 1822-1842 | Nuclear localization signal |
| 1843-1875 | Linker sequence |
| 1876-2379 | Zinc finger 82862 DNA binding domain |
| 2380-2400 | ZFP-FokI LO linker |
| 2401-2973 | FokI KKR nuclease catalytic domain |
| 2974-2979 | Two translation stop codons |
| 2980-2986 | Linker sequence |
| 2987-3270 | 3′UTR element |
| 3271-3284 | Linker sequence |
| 3285-3244 | Poly(A) tail |
| 3245-3450 | Linker sequence |
| 3451-3675 | Bovine growth hormone poly A signal |
| 3676-3817 | Linker sequence |
| 3818-4642 | Kanamycin resistant gene and promoter |
| 4643-4940 | Linker sequence |
| 4941-5614 | pUC origin |
| 5615-5620 | Linker sequence |

TABLE 18
Complete ZFN protein sequence translated from the Target Site B Vector
I plasmid. The first ZFNs (SEQ ID NO: 49) and the second ZFN (SEQ ID NO: 50)
(as separated by “//”) are bicistronically expressed in cells via
the 2A peptide.

MDYKDHDGDYKDHDIDYKDDDDKMAPKKKRKVGIHGVPAAMGQLVKSELEEKKSELRHKLKYVPHEYIELIEIARN
STQDRILEMKVMEFFMKVYGYRGKHLGGSRKPDGAIYTVGSPIDYGVIVDTKAYSGGYNLPIGQADEMERYVEENQ
TRDKHLNPNEWWKVYPSSVTEFKFLFVSGHFKGNYKAQLTRLNHITNCNGAVLSVEELLIGGEMIKAGTLTLEEVR
RKFNNGEINFSGAQGSTLDFRPFQCRICMRNFSRPYTLRLHIRTHTGEKPFACDICGRKFARSANLTRHTKIHTGS
QKPFQCRICMRNFSRSDALSTHIRTHTGEKPFACDICGRKFADRSTRTKHTKIHTGEKPFQCRICMRKFADRSTRT
KHTKIHLRQKDRSGGGEGRGSLLTCGDVEENPG//PRTMDYKDHDGDYKDHDIDYKDDDDKMAPKKKRKVGIHGVP
AAMAERPFQCRICMQNFSRSDVLSAHIRTHTGEKPFACDICGKKFADRSNRIKHTKIHTGSQKPFQCRICMQNFSD
RSHLTRHIRTHTGEKPFACDICGRKFALKQHLTRHTKIHTGEKPFQCRICMQNFSQSGNLARHIRTHTGEKPFACD
ICGRKFAQSTPRTTHTKIHLRGSQLVKSELEEKKSELRHKLKYVPHEYIELIEIARNSTQDRILEMKVMEFFMKVY
GYRGKHLGGSRKPDGAIYTVGSPIDYGVIVDTKAYSGGYNLPIGQADEMQRYVKENQTRNKHINPNEWWKVYPSSV
TEFKFLFVSGHFKGNYKAQLTRLNRKTNCNGAVLSVEELLIGGEMIKAGTLTLEEVRRKFNNGEINF

TABLE 19
Complete nucleotide sequence of Target Site G Vector I plasmid
(SEQ ID NO: 40)

GCTGCTTCGCGATGTACGGGCCAGATATACGCGCATGTTCTTTCCTGCGTTATCCCCTGATTCTGTGGATAACCGT
ATTACCGCCTTTGAGTGAGCTGATACCGCTCGCCGCAGCCGAACGACCGAGCGCAGCGAGTCAGTGAGCGAGGAAG
CGGAAGAGCGCCCAATACGCAAACCGCCTCTCCCCGCGCGTTGGCCGATTCATTAATGCAGCTGGCACGACAGGTT
TCCCGACTGGAAAGCGGGCAGTGAGCGCAACGCAATTAATGTGAGTTAGCTCACTCATTAGGCACCCCAGGCTTTA
CACTTTATGCTTCCGGCTCGTATGTTGTGTGGAATTGTGAGCGGATAACAATTTCACACAGGAAACAGCTATGACC
ATGATTACGCCAAGCTCTAATACGACTCACTATAGGGAGACAAGCTTGAATACAAGCTTGCTTGTTCTTTTTGCAG
AAGCTCAGAATAAACGCTCAACTTTGGCAGATCGAATTCGCCATGGACTACAAAGACCATGACGGTGATTATAAAG
ATCATGACATCGATTACAAGGATGACGATGACAAGATGGCCCCCAAGAAGAAGAGGAAGGTCGGCATCCACGGGGT
ACCCGCCGCTATGGGACAGCTGGTGAAGAGCGAGCTGGAGGAGAAGAAGTCCGAGCTGCGGCACAAGCTGAAGTAC
GTGCCCCACGAGTACATCGAGCTGATCGAGATCGCCAGGAACAGCACCCAGGACCGCATCCTGGAGATGAAGGTGA
TGGAGTTCTTCATGAAGGTGTACGGCTACAGGGGAAAGCACCTGGGCGGAAGCAGAAAGCCTGACGGCGCCATCTA
TACAGTGGGCAGCCCCATCGATTACGGCGTGATCGTGGACACAAAGGCCTACAGCGGCGGCTACAATCTGCCTATC
GGCCAGGCCGACGAGATGGAGAGATACGTGGAGGAGAACCAGACCCGGGATAAGCACCTCAACCCCAACGAGTGGT
GGAAGGTGTACCCTAGCAGCGTGACCGAGTTCAAGTTCCTGTTCGTGAGCGGCCACTTCAAGGGCAACTACAAGGC
CCAGCTGACCAGGCTGAACCACATCACCAACTGCAATGGCGCCGTGCTGAGCGTGGAGGAGCTGCTGATCGGCGGC
GAGATGATCAAAGCCGGCACCCTGACACTGGAGGAGGTGCGGCGCAAGTTCAACAACGGCGAGATCAACTTCAGCG
GCACTCCACACGAAGTGGGAGTGTACACACTTAGGCCCTTCCAGTGTCGAATCTGCATGCGTAACTTCAGTCGTAG
TGACCACCTGAGCCGGCACATCCGCACCCACACAGGCGAGAAGCCTTTTGCCTGTGACATTTGTGGGAGGAAATTT
GCCGACAGCAGCGACCGCAAAAAGCATACCAAGATACACACGGGCGAGAAGCCCTTCCAGTGTCGAATCTGCATGC
GTAACTTCAGTCGCTCCGACACCCTGTCCGAGCACATCCGCACCCACACCGGCGAGAAGCCTTTTGCCTGTGACAT
TTGTGGGAGGAAATTTGCCCAGTCCGGCGACCTGACCCGCCATACCAAGATACACACGCACCCGCGCGCCCCGATC
CCGAAGCCCTTCCAGTGTCGAATCTGCATGCGTAACTTCAGTCAGTCCTCCGACCTGTCCCGCCACATCCGCACCC
ACACCGGCGAGAAGCCTTTTGCCTGTGACATTTGTGGGAGGAAATTTGCCTACAAGTGGACCCTGCGCAACCATAC
CAAGATACACCTGCGGCAGAAGGACAGATCTGGCGGCGGAGAGGGCAGAGGAAGTCTTCTAACCTGCGGTGACGTG
GAGGAGAATCCCGGCCCTAGGACCATGGACTACAAAGACCATGACGGTGATTATAAAGATCATGACATCGATTACA
AGGATGACGATGACAAGATGGCCCCCAAGAAGAAGAGGAAGGTCGGCATTCATGGGGTACCCGCCGCTATGGGACA
GCTGGTGAAGAGCGAGCTGGAGGAGAAGAAGTCCGAGCTGCGGCACAAGCTGAAGTACGTGCCCCACGAGTACATC
GAGCTGATCGAGATCGCCAGGAACAGCACCCAGGACCGCATCCTGGAGATGAAGGTGATGGAGTTCTTCATGAAGG
TGTACGGCTACAGGGGAAAGCACCTGGGCGGAAGCAGAAAGCCTGACGGCGCCATCTATACAGTGGGCAGCCCCAT
CGATTACGGCGTGATCGTGGACACAAAGGCCTACAGCGGCGGCTACAATCTGCCTATCGGCCAGGCCGACGAGATG
CAGAGATACGTGAAGGAGAACCAGACCCGGAATAAGCACATCAACCCCAACGAGTGGTGGAAGGTGTACCCTAGCA
GCGTGACCGAGTTCAAGTTCCTGTTCGTGAGCGGCCACTTCAAGGGCAACTACAAGGCCCAGCTGACCAGGCTGAA
CCGCAAAACCAACTGCAATGGCGCCGTGCTGAGCGTGGAGGAGCTGCTGATCGGCGGCGAGATGATCAAAGCCGGC
ACCCTGACACTGGAGGAGGTGCGGCGCAAGTTCAACAACGGCGAGATCAACTTCAGCGGCACTCCACACGAAGTGG
GAGTGTACACACTTAGGCCCTTCCAGTGTCGAATCTGCATGCGTAACTTCAGTTCCAACCAGAACCTGACCACCCA
CATCCGCACCCACACCGGCGAGAAGCCTTTTGCCTGTGACATTTGTGGGAGGAAATTTGCCGACCGCTCCCACCTG
GCCCGCCATACCAAGATACACACGGGCGAGAAGCCCTTCCAGTGTCGAATCTGCATGCAGAAGTTTGCCCAGTCCG
GCGACCTGACCCGCCATACCAAGATACACACGGGCGAGAAGCCCTTCCAGTGTCGAATCTGCATGCAGAACTTCAG
TTGGAAGCACGACCTGACCAACCACATCCGCACCCACACCGGCGAGAAGCCTTTTGCCTGTGACATTTGTGGGAGG
AAATTTGCCACCTCCGGCAACCTGACCCGCCATACCAAGATACACCTGCGGCAGAAGGACTGATAACTCGAGTCTA
GAAGCTCGCTTTCTTGCTGTCCAATTTCTATTAAAGGTTCCTTTGTTCCCTAAGTCCAACTACTAAACTGGGGGAT
ATTATGAAGGGCCTTGAGCATCTGGATTCTGCCTAATAAAAAACATTTATTTTCATTGCTGCGCTAGAAGCTCGCT
TTCTTGCTGTCCAATTTCTATTAAAGGTTCCTTTGTTCCCTAAGTCCAACTACTAAACTGGGGGATATTATGAAGG
GCCTTGAGCATCTGGATTCTGCCTAATAAAAAACATTTATTTTCATTGCTGCGGGACATTCTTAATTAAAAAAAAA
AAAAAAAAAAAAAAAAAAAAAAAAAAAAAAAAAAAAAAAAAAAAAAAAAAACTAGTGGCGCCTGATGCGGTATTTT
CTCCTTACGCATCTGTGCGGTATTTCACACCGCATAATCCAGCACAGTGGCGGCCCGTTTAAACCCGCTGATCAGC
CTCGACTGTGCCTTCTAGTTGCCAGCCATCTGTTGTTTGCCCCTCCCCCGTGCCTTCCTTGACCCTGGAAGGTGCC
ACTCCCACTGTCCTTTCCTAATAAAATGAGGAAATTGCATCGCATTGTCTGAGTAGGTGTCATTCTATTCTGGGGG
GTGGGGTGGGGCAGGACAGCAAGGGGGAGGATTGGGAAGACAATAGCAGGCATGCTGGGGATGCGGTGGGCTCTAT
GGCTTCTACTGGGCGGTTTTATGGACAGCAAGCGAACCGGAATTGCCAGCTGGGGCGCCCTCTGGTAAGGTTGGGA
AGCCCTGCAAAGTAAACTGGATGGCTTTCTTGCCGCCAAGGATCTGATGGCGCAGGGGATCAAGCTCTGATCAAGA
GACAGGATGAGGATCGTTTCGCATGATTGAACAAGATGGATTGCACGCAGGTTCTCCGGCCGCTTGGGTGGAGAGG
CTATTCGGCTATGACTGGGCACAACAGACAATCGGCTGCTCTGATGCCGCCGTGTTCCGGCTGTCAGCGCAGGGGC
GCCCGGTTCTTTTTGTCAAGACCGACCTGTCCGGTGCCCTGAATGAACTGCAAGACGAGGCAGCGCGGCTATCGTG
GCTGGCCACGACGGGCGTTCCTTGCGCAGCTGTGCTCGACGTTGTCACTGAAGCGGGAAGGGACTGGCTGCTATTG
GGCGAAGTGCCGGGGCAGGATCTCCTGTCATCTCACCTTGCTCCTGCCGAGAAAGTATCCATCATGGCTGATGCAA
TGCGGCGGCTGCATACGCTTGATCCGGCTACCTGCCCATTCGACCACCAAGCGAAACATCGCATCGAGCGAGCACG
TACTCGGATGGAAGCCGGTCTTGTCGATCAGGATGATCTGGACGAAGAGCATCAGGGGCTCGCGCCAGCCGAACTG
TTCGCCAGGCTCAAGGCGAGCATGCCCGACGGCGAGGATCTCGTCGTGACCCATGGCGATGCCTGCTTGCCGAATA
TCATGGTGGAAAATGGCCGCTTTTCTGGATTCATCGACTGTGGCCGGCTGGGTGTGGCGGACCGCTATCAGGACAT
AGCGTTGGCTACCCGTGATATTGCTGAAGAGCTTGGCGGCGAATGGGCTGACCGCTTCCTCGTGCTTTACGGTATC
GCCGCTCCCGATTCGCAGCGCATCGCCTTCTATCGCCTTCTTGACGAGTTCTTCTGAATTATTAACGCTTACAATT
TCCTGATGCGGTATTTTCTCCTTACGCATCTGTGCGGTATTTCACACCGCATCAGGTGGCACTTTTCGGGGAAATG
TGCGCGGAACCCCTATTTGTTTATTTTTCTAAATACATTCAAATATGTATCCGCTCATGAGACAATAACCCTGATA
AATGCTTCAATAATAGCACGTGCTAAAACTTCATTTTTAATTTAAAAGGATCTAGGTGAAGATCCTTTTTGATAAT
CTCATGACCAAAATCCCTTAACGTGAGTTTTCGTTCCACTGAGCGTCAGACCCCGTAGAAAAGATCAAAGGATCTT
CTTGAGATCCTTTTTTTCTGCGCGTAATCTGCTGCTTGCAAACAAAAAAACCACCGCTACCAGCGGTGGTTTGTTT
GCCGGATCAAGAGCTACCAACTCTTTTTCCGAAGGTAACTGGCTTCAGCAGAGCGCAGATACCAAATACTGTTCTT
CTAGTGTAGCCGTAGTTAGGCCACCACTTCAAGAACTCTGTAGCACCGCCTACATACCTCGCTCTGCTAATCCTGT
TACCAGTGGCTGCTGCCAGTGGCGATAAGTCGTGTCTTACCGGGTTGGACTCAAGACGATAGTTACCGGATAAGGC
GCAGCGGTCGGGCTGAACGGGGGGTTCGTGCACACAGCCCAGCTTGGAGCGAACGACCTACACCGAACTGAGATAC
CTACAGCGTGAGCTATGAGAAAGCGCCACGCTTCCCGAAGGGAGAAAGGCGGACAGGTATCCGGTAAGCGGCAGGG
TCGGAACAGGAGAGCGCACGAGGGAGCTTCCAGGGGGAAACGCCTGGTATCTTTATAGTCCTGTCGGGTTTCGCCA
CCTCTGACTTGAGCGTCGATTTTTGTGATGCTCGTCAGGGGGGCGGAGCCTATGGAAAAACGCCAGCAACGCGGCC
TTTTTACGGTTCCTGGCCTTTTGCTGGCCTTTTGCTCACATGTTCTT

TABLE 20
Features of the CHITA ZEN plasmid Target Site G Vector I

| Nucleotide Position | Region/Open Reading Fram |
|---|---|
| 1-33 | Linker sequence |
| 34-397 | Linker sequence |
| 398-417 | T7 promoter/primer site |
| 415 | T7 transcription start site |
| 418-438 | Linker sequence |
| 439-489 | 5′UTR element |
| 490-498 | Linker sequence |
| 499-501 | Translation start codon |
| 502-567 | Hydrophilic peptide |
| 568-573 | Linker sequence |
| 574-594 | Nuclear localization signal |
| 595-624 | Linker sequence |
| 625-1212 | FokI ELD nuclease catalytic domain |
| 1213-1248 | FokI-ZFP N7a linker |
| 1249-1773 | Zinc finger 87254 DNA binding domain |
| 1774-1779 | Linker sequence |
| 1780-1842 | 2A self-cleaving peptide |
| 1843-1836 | Linker sequence |
| 1837-1917 | Hydrophilic peptide |
| 1918-1923 | Linker sequence |
| 1924-1944 | Nuclear localization signal |
| 1945-1974 | Linker sequence |
| 1975-2562 | FokI KKR nuclease catalytic domain |
| 2563-2598 | FokI-ZFP N7a linker |
| 2599-3024 | Zinc finger 84221 DNA binding domain |
| 3025-3030 | Translation stop codon |
| 3031-3037 | Linker sequence |
| 3038-3321 | 3′UTR element |
| 3322-3335 | Linker sequence |
| 3336-3395 | Poly(A) tail |
| 3396-3501 | Linker sequence |
| 3502-3726 | Bovine growth hormone poly A signal |
| 3727-3898 | Linker sequence |
| 3899-4693 | Kanamycin resistant gene and promoter |
| 4694-4991 | Linker sequence |
| 4992-5665 | pUC origin |
| 5666-5671 | Linker sequence |

TABLE 21
Complete ZFN protein sequence translated from the Target Site G Vector
I plasmid. The first ZFNs (SEQ ID NO: 51) and the second ZFN (SEQ ID NO: 52)
(as separated by “//”) are bicistronically expressed in cells via
the 2A peptide.

MDYKDHDGDYKDHDIDYKDDDDKMAPKKKRKVGIHGVPAAMGQLVKSELEEKKSELRHKLKYVPHEYIELIEIARN
STQDRILEMKVMEFFMKVYGYRGKHLGGSRKPDGAIYTVGSPIDYGVIVDTKAYSGGYNLPIGQADEMERYVEENQ
TRDKHLNPNEWWKVYPSSVTEFKFLFVSGHFKGNYKAQLTRINHITNCNGAVLSVEELLIGGEMIKAGTLTLEEVR
RKFNNGEINFSGTPHEVGVYTLRPFQCRICMRNFSRSDHLSRHIRTHTGEKPFACDICGRKFADSSDRKKHTKIHT
GEKPFQCRICMRNFSRSDTLSEHIRTHTGEKPFACDICGRKFAQSGDLTRHTKIHTHPRAPIPKPFQCRICMRNFS
QSSDLSRHIRTHTGEKPFACDICGRKFAYKWTLRNHTKIHLROKDRSGGGEGRGSLLTCGDVEENPG//PRTMDYK
DHDGDYKDHDIDYKDDDDKMAPKKKRKVGIHGVPAAMGQLVKSELEEKKSELRHKLKYVPHEYIELIEIARNSTQD
RILEMKVMEFFMKVYGYRGKHLGGSRKPDGAIYTVGSPIDYGVIVDTKAYSGGYNLPIGQADEMQRYVKENQTRNK
HINPNEWWKVYPSSVTEFKFLFVSGHFKGNYKAQLTRLNRKTNCNGAVLSVEELLIGGEMIKAGTLTLEEVRRKFN
NGEINFSGTPHEVGVYTLRPFQCRICMRNFSSNQNLTTHIRTHTGEKPFACDICGRKFADRSHLARHTKIHTGEKP
FQCRICMQKFAQSGDLTRHTKIHTGEKPFQCRICMQNFSWKHDLTNHIRTHTGEKPFACDICGRKFATSGNLTRHT
KIHLRQKD

TABLE 22
Complete ZFN protein sequence translated from the Target Site G Vector
II plasmid (SEQ ID NO: 57) and corresponding nucleotide sequence.
The first ZFN 87278 (SEQ ID NO: 54) and the second ZFN 87232 (SEQ ID NO: 56)
are bicistronically expressed in cells via the 2A peptide.

| | |
|---|---|
| ZFN CIITA 87278 | ATGGACTACAAAGACCATGACGGTGATTATAAAGATCATGACATCGATTACAAGGATG |
| DNA (SEQ ID NO: | ACGATGACAAGATGGCCCCCAAGAAGAAGAGGAAGGTCGGCATCCACGGGGTACCCGC |
| 53) | CGCTATGGGACAGCTGGTGAAGAGCGAGCTGGAGGAGAAGAAGTCCGAGCTGCGGCAC |
| | AAGCTGAAGTACGTGCCCCACGAGTACATCGAGCTGATCGAGATCGCCAGGAACAGCA |
| | CCCAGGACCGCATCCTGGAGATGAAGGTGATGGAGTTCTTCATGAAGGTGTACGGCTA |
| | CAGGGGAAAGCACCTGGGCGGAAGCAGAAAGCCTGACGGCGCCATCTATACAGTGGGC |
| | AGCCCCATCGATTACGGCGTGATCGTGGACACAAAGGCCTACAGCGGCGGCTACAATC |
| | TGCCTATCGGCCAGGCCGACGAGATGGAGAGATACGTGGAGGAGAACCAGACCCGGGA |
| | TAAGCACCTCAACCCCAACGAGTGGTGGAAGGTGTACCCTAGCAGCGTGACCGAGTTC |
| | AAGTTCCTGTTCGTGAGCGGCCACTTCAGCGGCAACTACAAGGCCCAGCTGACCAGGC |
| | TGAACCACATCACCAACTGCAATGGCGCCGTGCTGAGCGTGGAGGAGCTGCTGATCGG |
| | CGGCGAGATGATCAAAGCCGGCACCCTGACACTGGAGGAGGTGCGGCGCAAGTTCAAC |
| | AACGGCGAGATCAACTTCAGCGGCACTCCACACGAAGTGGGAGTGTACACACTTAGGC |
| | CCTTCCAGTGTCGAATCTGCATGCGTAACTTCAGTCGTAGTGACCACCTGAGCCGGCA |
| | CATCCGCACCCACACAGGCGAGAAGCCTTTTGCCTGTGACATTTGTGGGAGGAAATTT |
| | GCCGACAGCAGCGACCGCAAAAAGCATACCAAGATACACACGGGCGAGAAGCCCTTCC |
| | AGTGTCGAATCTGCATGCGTAACTTCAGTCGCTCCGACACCCTGTCCGAGCACATCCG |
| | CACCCACACCGGCGAGAAGCCTTTTGCCTGTGACATTTGTGGGAGGAAATTTGCCCAG |
| | TCCGGCGACCTGACCCGCCATACCAAGATACACACGCACCCGCGCGCCCCGATCCCGA |
| | AGCCCTTCCAGTGTCGAATCTGCATGCGTAACTTCAGTCAGTCCTCCGACCTGTCCCG |
| | CCACATCCGCACCCACACCGGCGAGAAGCCTTTTGCCTGTGACATTTGTGGGAGGAAA |
| | TTTGCCTACAAGTGGACCCTGCGCAACCATACCAAGATACACCTGCGGCAGAAGGAC |
| ZFN CIITA 87278 | MDYKDHDGDYKDHDIDYKDDDDKMAPKKKRKVGIHGVPAAMGQLVKSELEEKKSELRH |
| protein (SEQ ID | KLKYVPHEYIELIEIARNSTQDRILEMKVMEFFMKVYGYRGKHLGGSRKPDGAIYTVG |
| NO: 54) | SPIDYGVIVDTKAYSGGYNLPIGQADEMERYVEENQTRDKHLNPNEWWKVYPSSVTEF |
| | KFLFVSGHFSGNYKAQLTRINHITNCNGAVLSVEELLIGGEMIKAGTLTLEEVRRKFN |
| | NGEINFSGTPHEVGVYTLRPFQCRICMRNFSRSDHLSRHIRTHTGEKPFACDICGRKF |
| | ADSSDRKKHTKIHTGEKPFQCRICMRNFSRSDTLSEHIRTHTGEKPFACDICGRKFAQ |
| | SGDLTRHTKIHTHPRAPIPKPFQCRICMRNFSQSSDLSRHIRTHTGEKPFACDICGRK |
| | FAYKWTLRNHTKIHLRQKD |
| CIITA 87232 DNA | CAGCTGGTGAAGAGCGAGCTGGAGGAGAAGAAGTCCGAGCTGCGGCACAAGCTGAAGT |
| (SEQ ID NO: 55) | ACGTGCCCCACGAGTACATCGAGCTGATCGAGATCGCCAGGAACAGCACCCAGGACCG |
| | CATCCTGGAGATGAAGGTGATGGAGTTCTTCATGAAGGTGTACGGCTACAGGGGAAAG |
| | CACCTGGGCGGAAGCAGAAAGCCTGACGGCGCCATCTATACAGTGGGCAGCCCCATCG |
| | ATTACGGCGTGATCGTGGACACAAAGGCCTACAGCGGCGGCTACAATCTGCCTACCGG |
| | CCAGGCCGACGAGATGCAGAGATACGTGAAGGAGAACCAGACCCGGAATAAGCACATC |
| | AACCCCAACGAGTGGTGGAAGGTGTACCCTAGCAGCGTGACCGAGTTCAAGTTCCTGT |
| | TCGTGAGCGGCCACTTCAAGGGCAACTACAAGGCCCAGCTGACCAGGCTGAACCGCAA |
| | AACCAACTGCAATGGCGCCGTGCTGAGCGTGGAGGAGCTGCTGATCGGCGGCGAGATG |
| | ATCAAAGCCGGCACCCTGACACTGGAGGAGGTGCGGCGCAAGTTCAACAACGGCGAGA |
| | TCAACTTCAGCGGCACTCCACACGAAGTGGGAGTGTACACACTTAGGCCCTTCCAGTG |
| | TCGAATCTGCATGCGTAACTTCAGTTCCAACCAGAACCTGACCACCCACATCCGCACC |
| | CACACCGGCGAGAAGCCTTTTGCCTGTGACATTTGTGGGAGGAAATTTGCCGACCGCT |
| | CCCACCTGGCCCGCCATACCAAGATACACACGGGCGAGAAGCCCTTCCAGTGTCGAAT |
| | CTGCATGCAGAAGTTTGCCCAGTCCGGCGACCTGACCCGCCATACCAAGATACACACG |
| | GGCGAGAAGCCCTTCCAGTGTCGAATCTGCATGCAGAACTTCAGTTGGAAGCACGACC |
| | TGACCAACCACATCCGCACCCACACCGGCGAGAAGCCTTTTGCCTGTGACATTTGTGG |
| | GAGGAAATTTGCCACCTCCGGCAACCTGACCCGCCATACCAAGATACACCTGCGGCAG |
| | AAGGACTGA |
| ZFN CIITA 87232 | QLVKSELEEKKSELRHKLKYVPHEYIELIEIARNSTODRILEMKVMEFFMKVYGYRGK |
| protein (SEQ ID | HLGGSRKPDGAIYTVGSPIDYGVIVDTKAYSGGYNLPTGQADEMQRYVKENQTRNKHI |
| NO: 56) | NPNEWWKVYPSSVTEFKFLFVSGHFKGNYKAQLTRLNRKTNCNGAVLSVEELLIGGEM |
| | IKAGTLTLEEVRRKFNNGEINFSGTPHEVGVYTLRPFQCRICMRNFSSNQNLTTHIRT |
| | HTGEKPFACDICGRKFADRSHLARHTKIHTGEKPFQCRICMQKFAQSGDLTRHTKIHT |
| | GEKPFQCRICMQNFSWKHDLTNHIRTHTGEKPFACDICGRKFATSGNLTRHTKIHLRQ |
| | KD |
| Target Site G | GCTGCTTCGCGATGTACGGGCCAGATATACGCGCATGTTCTTTCCTGCGTTATCCCCT |
| Vector II | GATTCTGTGGATAACCGTATTACCGCCTTTGAGTGAGCTGATACCGCTCGCCGCAGCC |
| plasmid (SEQ ID | GAACGACCGAGCGCAGCGAGTCAGTGAGCGAGGAAGCGGAAGAGCGCCCAATACGCAA |
| NO: 57) | ACCGCCTCTCCCCGCGCGTTGGCCGATTCATTAATGCAGCTGGCACGACAGGTTTCCC |
| | GACTGGAAAGCGGGCAGTGAGCGCAACGCAATTAATGTGAGTTAGCTCACTCATTAGG |
| | CACCCCAGGCTTTACACTTTATGCTTCCGGCTCGTATGTTGTGTGGAATTGTGAGCGG |
| | ATAACAATTTCACACAGGAAACAGCTATGACCATGATTACGCCAAGCTCTAATACGAC |
| | TCACTATAGGGAGACAAGCTTGAATACAAGCTTGCTTGTTCTTTTTGCAGAAGCTCAG |
| | AATAAACGCTCAACTTTGGCAGATCGAATTCGCCATGGACTACAAAGACCATGACGGT |
| | GATTATAAAGATCATGACATCGATTACAAGGATGACGATGACAAGATGGCCCCCAAGA |
| | AGAAGAGGAAGGTCGGCATCCACGGGGTACCCGCCGCTATGGGACAGCTGGTGAAGAG |
| | CGAGCTGGAGGAGAAGAAGTCCGAGCTGCGGCACAAGCTGAAGTACGTGCCCCACGAG |
| | TACATCGAGCTGATCGAGATCGCCAGGAACAGCACCCAGGACCGCATCCTGGAGATGA |
| | AGGTGATGGAGTTCTTCATGAAGGTGTACGGCTACAGGGGAAAGCACCTGGGCGGAAG |
| | CAGAAAGCCTGACGGCGCCATCTATACAGTGGGCAGCCCCATCGATTACGGCGTGATC |
| | GTGGACACAAAGGCCTACAGCGGCGGCTACAATCTGCCTATCGGCCAGGCCGACGAGA |
| | TGGAGAGATACGTGGAGGAGAACCAGACCCGGGATAAGCACCTCAACCCCAACGAGTG |
| | GTGGAAGGTGTACCCTAGCAGCGTGACCGAGTTCAAGTTCCTGTTCGTGAGCGGCCAC |
| | TTCAGCGGCAACTACAAGGCCCAGCTGACCAGGCTGAACCACATCACCAACTGCAATG |
| | GCGCCGTGCTGAGCGTGGAGGAGCTGCTGATCGGCGGCGAGATGATCAAAGCCGGCAC |
| | CCTGACACTGGAGGAGGTGCGGCGCAAGTTCAACAACGGCGAGATCAACTTCAGCGGC |
| | ACTCCACACGAAGTGGGAGTGTACACACTTAGGCCCTTCCAGTGTCGAATCTGCATGC |
| | GTAACTTCAGTCGTAGTGACCACCTGAGCCGGCACATCCGCACCCACACAGGCGAGAA |
| | GCCTTTTGCCTGTGACATTTGTGGGAGGAAATTTGCCGACAGCAGCGACCGCAAAAAG |
| | CATACCAAGATACACACGGGCGAGAAGCCCTTCCAGTGTCGAATCTGCATGCGTAACT |
| | TCAGTCGCTCCGACACCCTGTCCGAGCACATCCGCACCCACACCGGCGAGAAGCCTTT |
| | TGCCTGTGACATTTGTGGGAGGAAATTTGCCCAGTCCGGCGACCTGACCCGCCATACC |
| | AAGATACACACGCACCCGCGCGCCCCGATCCCGAAGCCCTTCCAGTGTCGAATCTGCA |
| | TGCGTAACTTCAGTCAGTCCTCCGACCTGTCCCGCCACATCCGCACCCACACCGGCGA |
| | GAAGCCTTTTGCCTGTGACATTTGTGGGAGGAAATTTGCCTACAAGTGGACCCTGCGC |
| | AACCATACCAAGATACACCTGCGGCAGAAGGACAGATCTGGCGGCGGAGAGGGCAGAG |
| | GAAGTCTTCTAACCTGCGGTGACGTGGAGGAGAATCCCGGCCCTAGGACCATGGACTA |
| | CAAAGACCATGACGGTGATTATAAAGATCATGACATCGATTACAAGGATGACGATGAC |
| | AAGATGGCCCCCAAGAAGAAGAGGAAGGTCGGCATTCATGGGGTACCCGCCGCTATGG |
| | GACAGCTGGTGAAGAGCGAGCTGGAGGAGAAGAAGTCCGAGCTGCGGCACAAGCTGAA |
| | GTACGTGCCCCACGAGTACATCGAGCTGATCGAGATCGCCAGGAACAGCACCCAGGAC |
| | CGCATCCTGGAGATGAAGGTGATGGAGTTCTTCATGAAGGTGTACGGCTACAGGGGAA |
| | AGCACCTGGGCGGAAGCAGAAAGCCTGACGGCGCCATCTATACAGTGGGCAGCCCCAT |
| | CGATTACGGCGTGATCGTGGACACAAAGGCCTACAGCGGCGGCTACAATCTGCCTACC |
| | GGCCAGGCCGACGAGATGCAGAGATACGTGAAGGAGAACCAGACCCGGAATAAGCACA |
| | TCAACCCCAACGAGTGGTGGAAGGTGTACCCTAGCAGCGTGACCGAGTTCAAGTTCCT |
| | GTTCGTGAGCGGCCACTTCAAGGGCAACTACAAGGCCCAGCTGACCAGGCTGAACCGC |
| | AAAACCAACTGCAATGGCGCCGTGCTGAGCGTGGAGGAGCTGCTGATCGGCGGCGAGA |
| | TGATCAAAGCCGGCACCCTGACACTGGAGGAGGTGCGGCGCAAGTTCAACAACGGCGA |
| | GATCAACTTCAGCGGCACTCCACACGAAGTGGGAGTGTACACACTTAGGCCCTTCCAG |
| | TGTCGAATCTGCATGCGTAACTTCAGTTCCAACCAGAACCTGACCACCCACATCCGCA |
| | CCCACACCGGCGAGAAGCCTTTTGCCTGTGACATTTGTGGGAGGAAATTTGCCGACCG |
| | CTCCCACCTGGCCCGCCATACCAAGATACACACGGGCGAGAAGCCCTTCCAGTGTCGA |
| | ATCTGCATGCAGAAGTTTGCCCAGTCCGGCGACCTGACCCGCCATACCAAGATACACA |
| | CGGGCGAGAAGCCCTTCCAGTGTCGAATCTGCATGCAGAACTTCAGTTGGAAGCACGA |
| | CCTGACCAACCACATCCGCACCCACACCGGCGAGAAGCCTTTTGCCTGTGACATTTGT |
| | GGGAGGAAATTTGCCACCTCCGGCAACCTGACCCGCCATACCAAGATACACCTGCGGC |
| | AGAAGGACTGATAACTCGAGTCTAGAAGCTCGCTTTCTTGCTGTCCAATTTCTATTAA |
| | AGGTTCCTTTGTTCCCTAAGTCCAACTACTAAACTGGGGGATATTATGAAGGGCCTTG |
| | AGCATCTGGATTCTGCCTAATAAAAAACATTTATTTTCATTGCTGCGCTAGAAGCTCG |
| | CTTTCTTGCTGTCCAATTTCTATTAAAGGTTCCTTTGTTCCCTAAGTCCAACTACTAA |
| | ACTGGGGGATATTATGAAGGGCCTTGAGCATCTGGATTCTGCCTAATAAAAAACATTT |
| | ATTTTCATTGCTGCGGGACATTCTTAATTAAAAAAAAAAAAAAAAAAAAAAAAAAAAA |
| | AAAAAAAAAAAAAAAAAAAAAAAAAAAAAAACTAGTGGCGCCTGATGCGGTATTTTCT |
| | CCTTACGCATCTGTGCGGTATTTCACACCGCATAATCCAGCACAGTGGCGGCCCGTTT |
| | AAACCCGCTGATCAGCCTCGACTGTGCCTTCTAGTTGCCAGCCATCTGTTGTTTGCCC |
| | CTCCCCCGTGCCTTCCTTGACCCTGGAAGGTGCCACTCCCACTGTCCTTTCCTAATAA |
| | AATGAGGAAATTGCATCGCATTGTCTGAGTAGGTGTCATTCTATTCTGGGGGGTGGGG |
| | TGGGGCAGGACAGCAAGGGGGAGGATTGGGAAGACAATAGCAGGCATGCTGGGGATGC |
| | GGTGGGCTCTATGGCTTCTACTGGGCGGTTTTATGGACAGCAAGCGAACCGGAATTGC |
| | CAGCTGGGGCGCCCTCTGGTAAGGTTGGGAAGCCCTGCAAAGTAAACTGGATGGCTTT |
| | CTTGCCGCCAAGGATCTGATGGCGCAGGGGATCAAGCTCTGATCAAGAGACAGGATGA |
| | GGATCGTTTCGCATGATTGAACAAGATGGATTGACGCAGGTTCTCCGGCCGCTTGGGT |
| | GGAGAGGCTATTCGGCTATGACTGGGCACAACAGACAATCGGCTGCTCTGATGCCGCC |
| | GTGTTCCGGCTGTCAGCGCAGGGGCGCCCGGTTCTTTTTGTCAAGACCGACCTGTCCG |
| | GTGCCCTGAATGAACTGCAAGACGAGGCAGCGCGGCTATCGTGGCTGGCCACGACGGG |
| | CGTTCCTTGCGCAGCTGTGCTCGACGTTGTCACTGAAGCGGGAAGGGACTGGCTGCTA |
| | TTGGGCGAAGTGCCGGGGCAGGATCTCCTGTCATCTCACCTTGCTCCTGCCGAGAAAG |
| | TATCCATCATGGCTGATGCAATGCGGCGGCTGCATACGCTTGATCCGGCTACCTGCCC |
| | ATTCGACCACCAAGCGAAACATCGCATCGAGCGAGCACGTACTCGGATGGAAGCCGGT |
| | CTTGTCGATCAGGATGATCTGGACGAAGAGCATCAGGGGCTCGCGCCAGCCGAACTGT |
| | TCGCCAGGCTCAAGGCGAGCATGCCCGACGGCGAGGATCTCGTCGTGACCCATGGCGA |
| | TGCCTGCTTGCCGAATATCATGGTGGAAAATGGCCGCTTTTCTGGATTCATCGACTGT |
| | GGCCGGCTGGGTGTGGCGGACCGCTATCAGGACATAGCGTTGGCTACCCGTGATATTG |
| | CTGAAGAGCTTGGCGGCGAATGGGCTGACCGCTTCCTCGTGCTTTACGGTATCGCCGC |
| | TCCCGATTCGCAGCGCATCGCCTTCTATCGCCTTCTTGACGAGTTCTTCTGAATTATT |
| | AACGCTTACAATTTCCTGATGCGGTATTTTCTCCTTACGCATCTGTGCGGTATTTCAC |
| | ACCGCATCAGGTGGCACTTTTCGGGGAAATGTGCGCGGAACCCCTATTTGTTTATTTT |
| | TCTAAATACATTCAAATATGTATCCGCTCATGAGACAATAACCCTGATAAATGCTTCA |
| | ATAATAGCACGTGCTAAAACTTCATTTTTAATTTAAAAGGATCTAGGTGAAGATCCTT |
| | TTTGATAATCTCATGACCAAAATCCCTTAACGTGAGTTTTCGTTCCACTGAGCGTCAG |
| | ACCCCGTAGAAAAGATCAAAGGATCTTCTTGAGATCCTTTTTTTCTGCGCGTAATCTG |
| | CTGCTTGCAAACAAAAAAACCACCGCTACCAGCGGTGGTTTGTTTGCCGGATCAAGAG |
| | CTACCAACTCTTTTTCCGAAGGTAACTGGCTTCAGCAGAGCGCAGATACCAAATACTG |
| | TTCTTCTAGTGTAGCCGTAGTTAGGCCACCACTTCAAGAACTCTGTAGCACCGCCTAC |
| | ATACCTCGCTCTGCTAATCCTGTTACCAGTGGCTGCTGCCAGTGGCGATAAGTCGTGT |
| | CTTACCGGGTTGGACTCAAGACGATAGTTACCGGATAAGGCGCAGCGGTCGGGCTGAA |
| | CGGGGGGTTCGTGCACACAGCCCAGCTTGGAGCGAACGACCTACACCGAACTGAGATA |
| | CCTACAGCGTGAGCTATGAGAAAGCGCCACGCTTCCCGAAGGGAGAAAGGCGGACAGG |
| | TATCCGGTAAGCGGCAGGGTCGGAACAGGAGAGCGCACGAGGGAGCTTCCAGGGGGAA |
| | ACGCCTGGTATCTTTATAGTCCTGTCGGGTTTCGCCACCTCTGACTTGAGCGTCGATT |
| | TTTGTGATGCTCGTCAGGGGGGCGGAGCCTATGGAAAAACGCCAGCAACGCGGCCTTT |
| | TTACGGTTCCTGGCCTTTTGCTGGCCTTTTGCTCACATGTTCTT |

### Example 4. Editing Activity of Zinc Finger Nucleases

### Editing Activity of the CIITA ZFNs, 87278 and 87232, was Evaluated.

### Peripheral Blood Mononuclear Cell (PBMC) and Regulatory T Cells (Treg Cells) Isolation

Treg cells were freshly isolated from buffy coats obtained from heathy volunteer bloods from EFS (Marseille, France). Briefly, PBMCs were purified one day after blood collection. Next, CD4⁺/CD25⁺/CD127^{Low} Treg cells were isolated by column-free immunomagnetic positive selection using EASYSEP™ Releasable RAPIDSPHERES™. Next, bound magnetic particles were removed from the EASYSEP™-isolated CD25⁺ cells, and CD127 expressing cells were depleted by immunomagnetic negative selection. The living CD4⁺/CD25⁺CD127^{Low} Treg cells were counted by PI staining and used for downstream applications.

### Treg Cell Culture

After cells isolation, cells were plated in a cell culture medium supplemented with L-glutamine (20 mM), Gentamicin (75 μg/mL), rhIL-2 (1000 U/mL) and anti CD3/CD28 coated-beads. After electroporation, cells were plated in the same cell culture medium supplemented with L-glutamine (20 mM), Gentamicin (75 μg/mL), rhIL-2 (1000 U/mL) and 5% Serum Replacement technology. At day 4 post-electroporation, cells were harvested, counted and replated in a fresh complete medium.

CIITA ZFN mRNA Production

DNA sequences encoding for the CIITA ZFNs (87278 and 87232) were cloned into the pVAX-GEM2UX plasmid. After plasmid linearization with SpeI, mRNA transcripts were produced using the mMessageMachine T7 ultra-Kit and subsequently isolated by Lithium Chloride purification. Finally, mRNA quality was assessed using a Bioanalyzer.

### Treg Cell Electroporation

0.5*10E+06 cells were collected and suspended in an electroporation buffer at a cell concentration of 20*1E+06 cells/mL. mRNA encoding for CIITA ZFN was then mixed with resuspended cells at the indicated concentrations. Next, samples were loaded in electroporation cassettes and electroporated according to the manufacturer's instructions. After electric shock, cells were recovered and plated in the described medium (see Treg cell culture section).

### Immuno-Phenotyping

Cells were stained with anti-MHCII antibodies in the dark at 4° C. for 20 minutes. After two washing steps, cells were resuspended in FACS buffer containing SYTOX blue which was used as mortality marker. Samples were analyzed by flow cytometry using a MACSQuant analyzer.

### Indels Detection Using Next-Generation Sequencing

CIITA-targeted region was PCR amplified from genomic DNA by single PCR. Generated PCR products were then barcoded and the levels of modification were determined by paired-end deep sequencing on an Illumina MiSeq sequencing system. Miseq data were processed and analyzed in-house. Primers used to amplify genomic DNA for indel quantification are provided in the Table 23 below.

TABLE 23
primers used to
amplify genomic CIITA-targeted region

| Primer | sequence |
|---|---|
| CIITA- | ACACGACGCTCTTCCGATCTNNNNTCCCCAG |
| forward | TACGACTTTGTCTTC (SEQ ID NO: 45) |
| CIITA- | GACGTGTGCTCTTCCGATCTTCAAGATGTGG |
| reverse | CTGAAAACCTC (SEQ ID NO: 46) |

### Experimental Plan

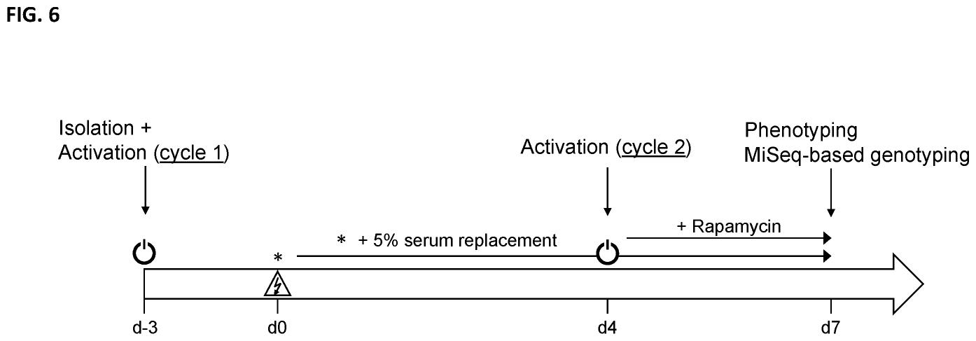

Freshly isolated CD4+/CD127Low/CD25+ Treg cells were activated using anti-CD3/CD28 beads (d-3). At day 0, ZFN-mRNA was electroporated into cells as indicated in the result section. After electroporation (EP), cells were cultures in presence of 5% serum replacement (SR). At day 4 post-EP, cells were re-activated by adding fresh anti-CD3/CD28 beads. Rapamycin was also added until day 7 post-EP, when cells were analyzed by immuno-phenotyping. DNA was also extracted to perform Miseq analysis. (see FIG. 6).

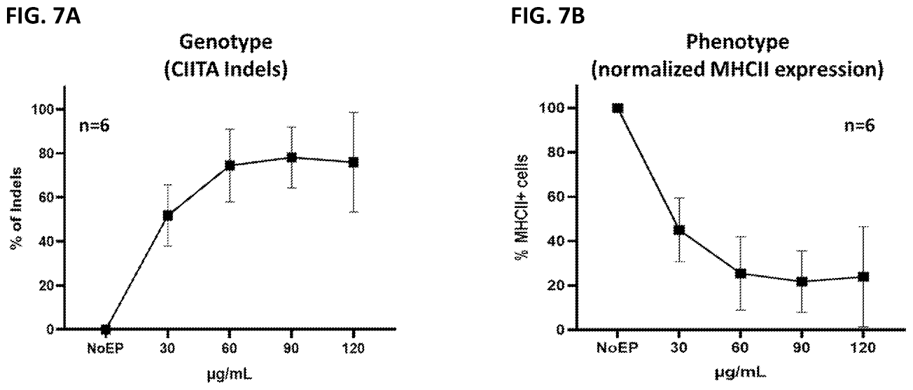

Treg cells were electroporated with different concentrations of CIITA ZFN mRNAs (0, 30, 60, 90 and 120 μg/mL). After one week, the editing efficiency of the CIITA ZFN was evaluated by next-generation sequencing (NGS). The optimal editing condition (Indels %) was obtained at 90 μg/mL of electroporated mRNA (FIG. 7A). In parallel, the immuno-phenotypic analysis of Treg cells has been performed to monitor the knock-out of the cell surface MHCII. Since MHCII expression fluctuates in time in Treg cells, data were normalized over the non-electroporated (NoEP) control condition. In line with the editing data, CIITA ZFN editing led to a strong MHCII knock-out with an optimal concentration of ZFN around 90 μg/mL (FIG. 7B).

All patents, patent applications and publications mentioned herein are hereby incorporated by reference in their entirety.

Although disclosure has been provided in some detail by way of illustration and example for the purposes of clarity of understanding, it will be apparent to those skilled in the art that various changes and modifications can be practiced without departing from the spirit or scope of the disclosure. Accordingly, the foregoing descriptions and examples should not be construed as limiting.

What is claimed is:

1. A polynucleotide comprising a nucleic acid sequence encoding a zinc finger nuclease (ZFN) that cleaves a CIITA gene, wherein the ZFN comprises a Zinc Finger DNA-binding domain that binds to a DNA sequence in the CIITA gene and a cleavage domain,
wherein the ZFN is capable of cleaving the CIITA gene between amino acids 28 and 29 corresponding to SEQ ID NO: 1 or between amino acids 461 and 462 corresponding to SEQ ID NO: 1.

2. The polynucleotide of claim 1, wherein the ZFN is capable of cleaving the CIITA gene between amino acids 28 and 29 corresponding to SEQ ID NO: 1.

3. The polynucleotide of claim 1, wherein ZFN is capable of cleaving the CIITA gene between amino acids 461 and 462 corresponding to SEQ ID NO: 1.

4. The polynucleotide of claim 1 or 2, wherein the DNA-binding domain binds to GCCACCATGGAGTTG (SEQ ID NO: 9) and/or CTAGAAGGTGGCTACCTG (SEQ ID NO:15).

5. The polynucleotide of claim 4, wherein the DNA-binding domain binds to GCCACCATGGAGTTG (SEQ ID NO: 9).

6. The polynucleotide of claim 4, wherein the DNA-binding domain binds to CTAGAAGGTGGCTACCTG (SEQ ID NO: 15).

7. The polynucleotide of claim 1 or 3, wherein the DNA-binding domain binds to ATTGCT and GAACCGTCCGGG (SEQ ID NO: 38) or GATCCTGCAGGCCAT (SEQ ID NO: 29).

8. The polynucleotide of claim 7, wherein the DNA-binding domain binds to ATTGCT and GAACCGTCCGGG (SEQ ID NO: 38).

9. The polynucleotide of claim 7, wherein the DNA-binding domain binds to GATCCTGCAGGCCAT (SEQ ID NO: 29).

10. The polynucleotide of claim 1, 2, 4, or 5, wherein the DNA-binding domain comprises five zinc fingers, which comprises finger 1 (F1) comprising SEQ ID NO: 10 [RPYTLRL], finger 2 (F2) comprising SEQ ID NO: 11 [RSANLTR], finger 3 (F3) comprising SEQ ID NO: 12 [RSDALST], finger 4 (F4) comprising SEQ ID NO: 13 [DRSTRTK], and finger 5 (F5) comprising SEQ ID NO: 14 [DRSTRTK].

11. The polynucleotide of claim 1, 2, 4, or 6, wherein the DNA-binding domain comprises six zinc fingers, which comprises F1 comprising SEQ ID NO: 16 [RSDVLSA], F2 comprising SEQ ID NO: 17 [DRSNRIK], F3 comprising SEQ ID NO: 18 [DRSHLTR], F4 comprising SEQ ID NO: 19 [LKQHLTR], F5 comprising SEQ ID NO: 20 [QSGNLAR], and F6 comprising SEQ ID NO: 21 [QSTPRTT].

12. The polynucleotide of any one of claims 1, 2, 4-6, 10, and 11, which encodes a zinc finger nuclease pair comprising a first zinc finger DNA-binding domain and a second zinc finger DNA-binding domain, wherein the first DNA-binding domain comprises five zinc fingers, which comprises finger 1 (F1) comprising SEQ ID NO: 10 [RPYTLRL], finger 2 (F2) comprising SEQ ID NO: 11 [RSANLTR], finger 3 (F3) comprising SEQ ID NO: 12 [RSDALST], finger 4 (F4) comprising 13 [DRSTRTK], and finger 5 (F5) comprising SEQ ID NO: 14 [DRSTRTK], and wherein the second DNA-binding domain comprises six zinc fingers, which comprises F1 comprising SEQ ID NO: 16 [RSDVLSA], F2 comprising SEQ ID NO: 17 [DRSNRIK], F3 comprising SEQ ID NO: 18 [DRSHLTR], F4 comprising SEQ ID NO: 19 [LKQHLTR], F5 comprising SEQ ID NO: 20 [QSGNLAR], and F6 comprising SEQ ID NO: 21 [QSTPRTT].

13. The polynucleotide of claims 1, 3, 7, and 8, wherein the DNA-binding domain comprises six zinc fingers, which comprises F1 comprising SEQ ID NO: 23 [RSDHLSR], F2 comprising SEQ ID NO: 24 [DSSDRKK], F3 comprising SEQ ID NO: 25 [RSDTLSE], F4 comprising 26 [QSGDLTR], and F5 comprising SEQ ID NO: 27 [QSSDLSR], and F6 comprising SEQ ID NO: 28 [YKWTLRN].

14. The polynucleotide of claims 1, 3, 7, and 9, wherein the DNA-binding domain comprises five zinc fingers, which comprises F1 comprising SEQ ID NO: 30 [SNQNLTT], F2 comprising SEQ ID NO: 31 [DRSHLAR], F3 comprising SEQ ID NO: 32 [QSGDLTR], F4 comprising SEQ ID NO: 33 [WKHDLTN], and F5 comprising SEQ ID NO: 34 [TSGNLTR].

15. The polynucleotide of any one of claims 1, 3, 7-9, 13, and 14, which encodes a zinc finger nuclease pair comprising a first zinc finger DNA-binding domain and a second zinc finger DNA-binding domain, wherein the first DNA-binding domain comprises six zinc fingers, which comprises F1 comprising SEQ ID NO: 23 [RSDHLSR], F2 comprising SEQ ID NO: 24 [DSSDRKK], F3 comprising SEQ ID NO: 25 [RSDTLSE], F4 comprising 26 [QSGDLTR], and F5 comprising SEQ ID NO: 27 [QSSDLSR], and F6 comprising SEQ ID NO: 28 [YKWTLRN], and wherein the second DNA-binding domain comprises five zinc fingers, which comprises F1 comprising SEQ ID NO: 30 [SNQNLTT], F2 comprising SEQ ID NO: 31 [DRSHLAR], F3 comprising SEQ ID NO: 32 [QSGDLTR], F4 comprising SEQ ID NO: 33 [WKHDLTN], and F5 comprising SEQ ID NO: 34 [TSGNLTR].

16. The polynucleotide of any one of claim 1, 3, or 7-9, wherein the DNA-binding domain comprises SEQ ID NO: 54.

17. The polynucleotide of any one of claim 1, 3, or 7-9, wherein the DNA-binding domain comprises SEQ ID NO: 56.

18. The polynucleotide of any one of claim 1, 3, 7-9, 16, or 17, which encodes a zinc finger nuclease pair comprising a first zinc finger DNA-binding domain and a second zinc finger DNA-binding domain, wherein the first DNA-binding domain comprises SEQ ID NO: 54 and the second DNA-binding domain comprises SEQ ID NO: 56.

19. The polynucleotide of any one of the preceding claims, wherein the cleavage domain comprises a FokI cleavage domain.

20. The polynucleotide any of claim 19, wherein the FokI cleavage domain further comprises one or more mutations at positions 418, 432, 441, 448, 476, 479, 481, 483, 486, 487, 490, 496, 499, 523, 525, 527, 537, 538 and 559 of SEQ ID NO 35.

21. The polynucleotide of claim 20, wherein the one or more mutations are at positions 479, 486, 496, 499, and/or 525.

22. The polynucleotide of claim 21, wherein the FokI cleavage domain comprise SEQ ID NO: 36 (FokELD)

23. The polynucleotide of claim 20, wherein the one or more mutations are at positions 490, 537, and/or 538.

24. The polynucleotide of claim 23, wherein the FokI cleavage domain comprise SEQ ID NO: 37 (FokKKR)

25. The polynucleotide of any one of claims 19 to 24, wherein the FokI cleavage domain forms a dimer prior to DNA cleavage.

26. The polynucleotide of claim 25, wherein the FokI dimer comprises a heterodimer.

27. The polynucleotide of claim 26, wherein the FokI heterodimer comprises a FokIELD dimer and a FokIKKR dimer.

28. The polynucleotide of any one of claims 1, 2, 4, 5, 10 and 12, wherein the ZFN comprises the amino acid sequence having at least about 70%, at least about 80%, at least about 85%, at least about 90%, at least about 95%, at least about 96%, at least about 97%, at least about 98%, at least about 99%, or about 100% sequence identity to SEQ ID NO: 5
(MDYKDHDGDYKDHDIDYKDDDDKMAPKKKRKVGIHGVPAAMGQLVKSEL
EEKKSELRHKLKYVPHEYIELIEIARNSTQDRILEMKVMEFFMKVYGYRG
KHLGGSRKPDGAIYTVGSPIDYGVIVDTKAYSGGYNLPIGQADEMERYVE
ENQTRDKHLNPNEWWKVYPSSVTEFKFLFVSGHFKGNYKAQLTRLNHITN
CNGAVLSVEELLIGGEMIKAGTLTLEEVRRKFNNGEINFSGAQGSTLDFR
PFQCRICMRNFSRPYTLRLHIRTHTGEKPFACDICGRKFARSANLTRHTK
IHTGSQKPFQCRICMRNFSRSDALSTHIRTHTGEKPFACDICGRKFADRS
TRTKHTKIHTGEKPFQCRICMRKFADRSTRTKHTKIHLRQKD.

29. The polynucleotide of any one of claims 1, 2, 4, 6, 11 and 12, wherein the ZFN comprises the amino acid sequence having at least about 70%, at least about 80%, at least about 85%, at least about 90%, at least about 95%, at least about 96%, at least about 97%, at least about 98%, at least about 99%, or about 100% SEQ sequence identity to ID NO: 6
(MDYKDHDGDYKDHDIDYKDDDDKMAPKKKRKVGIHGVPAAMAERPFQCR
ICMQNFSRSDVLSAHIRTHTGEKPFACDICGKKFADRSNRIKHTKIHTGS
QKPFQCRICMQNFSDRSHLTRHIRTHTGEKPFACDICGRKFALKQHLTRH
TKIHTGEKPFQCRICMQNFSQSGNLARHIRTHTGEKPFACDICGRKFAQS
TPRTTHTKIHLRGSQLVKSELEEKKSELRHKLKYVPHEYIELIEIARNST
QDRILEMKVMEFFMKVYGYRGKHLGGSRKPDGAIYTVGSPIDYGVIVDTK
AYSGGYNLPIGQADEMQRYVKENQTRNKHINPNEWWKVYPSSVTEFKFLF
VSGHFKGNYKAQLTRLNRKTNCNGAVLSVEELLIGGEMIKAGTLTLEEVR
RKFNNGEINF).

30. A polynucleotide comprising a sequence at least about 70%, at least about 80%, at least about 90%, at least about 95%, at least about 96%, at least about 97%, or least about 98%, at least about 99% or about 100% sequence identity to SEQ ID NO 39 [Complete Nucleotide Sequence of STV220-pVAX-GEM2UX-CIITA-B-76867-2A-82862 plasmid]

31. The polynucleotide of any one of claims 1, 3, 7, 8, 13, and 15, wherein the ZFN comprises the amino acid sequence having at least about 70%, at least about 80%, at least about 85%, at least about 90%, at least about 95%, at least about 96%, at least about 97%, at least about 98%, at least about 99%, or about 100% sequence identity to SEQ ID NO: 7
(MDYKDHDGDYKDHDIDYKDDDDKMAPKKKRKVGIHGVPAAMGQLVKSEL
EEKKSELRHKLKYVPHEYIELIEIARNSTQDRILEMKVMEFFMKVYGYRG
KHLGGSRKPDGAIYTVGSPIDYGVIVDTKAYSGGYNLPIGQADEMERYVE
ENQTRDKHLNPNEWWKVYPSSVTEFKFLFVSGHFKGNYKAQLTRLNHITN
CNGAVLSVEELLIGGEMIKAGTLTLEEVRRKFNNGEINFSGTPHEVGVYT
LRPFQCRICMRNFSRSDHLSRHIRTHTGEKPFACDICGRKFADSSDRKKH
TKIHTGEKPFQCRICMRNFSRSDTLSEHIRTHTGEKPFACDICGRKFAQS
GDLTRHTKIHTHPRAPIPKPFQCRICMRNFSQSSDLSRHIRTHTGEKPFA
CDICGRKFAYKWTLRNHTKIHLRQKD)
or
SEQ ID NO: 54
(MDYKDHDGDYKDHDIDYKDDDDKMAPKKKRKVGIHGVPAAMGQLVKSEL
EEKKSELRHKLKYVPHEYIELIEIARNSTQDRILEMKVMEFFMKVYGYRG
KHLGGSRKPDGAIYTVGSPIDYGVIVDTKAYSGGYNLPIGQADEMERYVE
ENQTRDKHLNPNEWWKVYPSSVTEFKFLFVSGHFSGNYKAQLTRLNHITN
CNGAVLSVEELLIGGEMIKAGTLTLEEVRRKFNNGEINFSGTPHEVGVYT
LRPFQCRICMRNFSRSDHLSRHIRTHTGEKPFACDICGRKFADSSDRKKH
TKIHTGEKPFQCRICMRNFSRSDTLSEHIRTHTGEKPFACDICGRKFAQS
GDLTRHTKIHTHPRAPIPKPFQCRICMRNFSQSSDLSRHIRTHTGEKPFA
CDICGRKFAYKWTLRNHTKIHLRQKD).

32. The polynucleotide of any one of claim 1, 3, 7, 8, 14 or 15, wherein the ZFN comprises the amino acid sequence having at least about 70%, at least about 80%, at least about 85%, at least about 90%, at least about 95%, at least about 96%, at least about 97%, at least about 98%, at least about 99%, or about 100% sequence identity to SEQ ID NO: 8
(MDYKDHDGDYKDHDIDYKDDDDKMAPKKKRKVGIHGVPAAMGQLVKSEL
EEKKSELRHKLKYVPHEYIELIEIARNSTQDRILEMKVMEFFMKVYGYRG
KHLGGSRKPDGAIYTVGSPIDYGVIVDTKAYSGGYNLPIGQADEMQRYVK
ENQTRNKHINPNEWWKVYPSSVTEFKFLFVSGHFKGNYKAQLTRLNRKTN
CNGAVLSVEELLIGGEMIKAGTLTLEEVRRKFNNGEINFSGTPHEVGVYT
LRPFQCRICMRNFSSNQNLTTHIRTHTGEKPFACDICGRKFADRSHLARH
TKIHTGEKPFQCRICMQKFAQSGDLTRHTKIHTGEKPFQCRICMQNFSWK
HDLTNHIRTHTGEKPFACDICGRKFATSGNLTRHTKIHLRQKD)
or
SEQ ID NO: 56
(QLVKSELEEKKSELRHKLKYVPHEYIELIEIARNSTQDRILEMKVMEFF
MKVYGYRGKHLGGSRKPDGAIYTVGSPIDYGVIVDTKAYSGGYNLPTGQA
DEMQRYVKENQTRNKHINPNEWWKVYPSSVTEFKFLFVSGHFKGNYKAQL
TRLNRKTNCNGAVLSVEELLIGGEMIKAGTLTLEEVRRKFNNGEINFSGT
PHEVGVYTLRPFQCRICMRNFSSNQNLTTHIRTHTGEKPFACDICGRKFA
DRSHLARHTKIHTGEKPFQCRICMQKFAQSGDLTRHTKIHTGEKPFQCRI
CMQNFSWKHDLTNHIRTHTGEKPFACDICGRKFATSGNLTRHTKIHLRQK
D).

33. A polynucleotide comprising a sequence at least about 70%, at least about 80%, at least about 90%, at least about 95%, at least about 96%, at least about 97%, or least about 98%, at least about 99% sequence identity to SEQ ID NO: 40, SEQ ID NO: 53, SEQ ID NO: 55, or SEQ ID NO: 57.

34. The polynucleotide of claims 1, 3, 7, and 8, wherein the DNA-binding domain comprises six zinc fingers, which comprises F1 comprising SEQ ID NO: 23 [RSDHLSR], F2 comprising SEQ ID NO: 24 [DSSDRKK], F3 comprising SEQ ID NO: 25 [RSDTLSE], F4 comprising 26 [QSGDLTR], and F5 comprising SEQ ID NO: 27 [QSSDLSR], and F6 comprising SEQ ID NO: 28 [YKWTLRN], wherein the cleavage domain comprises a FokI cleavage domain, and wherein the FokI cleavage domain further comprises K to S mutation at position 525 of SEQ ID NO 35.

35. The polynucleotide of claims 1, 3, 7, and 9, wherein the DNA-binding domain comprises five zinc fingers, which comprises F1 comprising SEQ ID NO: 30 [SNQNLTT], F2 comprising SEQ ID NO: 31 [DRSHLAR], F3 comprising SEQ ID NO: 32 [QSGDLTR], F4 comprising SEQ ID NO: 33 [WKHDLTN], and F5 comprising SEQ ID NO: 34 [TSGNLTR], wherein the cleavage domain comprises a FokI cleavage domain, and wherein the FokI cleavage domain further comprises I to T mutation at position 479 of SEQ ID NO 35.

36. A zinc finger nuclease (ZFN) that cleaves a CIITA gene, wherein the ZFN comprises a Zinc Finger DNA-binding domain that binds to a DNA sequence in the CIITA gene and a cleavage domain,
wherein the ZFN is capable of cleaving the CIITA gene between amino acids 28 and 29 corresponding to SEQ ID NO: 1 or between amino acids 461 and 462 corresponding to SEQ ID NO: 1.

37. The ZFN of claim 36, wherein the ZFN is capable of cleaving the CIITA gene between amino acids 28 and 29 corresponding to SEQ ID NO: 1.

38. The ZFN of claim 36, wherein the ZFN is capable of cleaving the CIITA gene between amino acids 461 and 462 corresponding to SEQ ID NO: 1.

39. The ZFN of claim 36 or 37, wherein the ZFP DNA-binding domain binds to GCCACCATGGAGTTG (SEQ ID NO: 9) and/or CTAGAAGGTGGCTACCTG (SEQ ID NO: 15).

40. The ZFN of claim 39, wherein the ZFP DNA-binding domain binds to GCCACCATGGAGTTG (SEQ ID NO: 9).

41. The ZFN of claim 39, wherein the ZFP DNA-binding domain binds to CTAGAAGGTGGCTACCTG (SEQ ID NO: 15).

42. The ZFN of claim 36 or 38, wherein the ZFP DNA-binding domain binds to ATTGCT and GAACCGTCCGGG (SEQ ID NO: 38) or GATCCTGCAGGCCAT (SEQ ID NO: 29).

43. The ZFN of claim 42, wherein the ZFP DNA-binding domain binds to ATTGCT and GAACCGTCCGGG (SEQ ID NO: 38).

44. The ZFN of claim 42, wherein the ZFP DNA-binding domain binds to GATCCTGCAGGCCAT (SEQ ID NO: 29).

45. The ZFN of any one of claims 36, 37, 39, and 40, wherein the ZFP DNA-binding domain comprises five zinc fingers, which comprises finger 1 (F1) comprising SEQ ID NO: 10 [RPYTLRL], finger 2 (F2) comprising SEQ ID NO: 11 [RSANLTR], finger 3 (F3) comprising SEQ ID NO: 12 [RSDALST], finger 4 (F4) comprising SEQ ID NO: 13 [DRSTRTK], and finger 5 (F5) comprising SEQ ID NO: 14 [DRSTRTK].

46. The ZFN of any one of claims 36, 37, 39, and 40, wherein the ZFP DNA-binding domain comprises six zinc fingers, which comprises F1 comprising SEQ ID NO: 16 [RSDVLSA], F2 comprising SEQ ID NO: 17 [DRSNRIK], F3 comprising SEQ ID NO: 18 [DRSHLTR], F4 comprising SEQ ID NO: 19 [LKQHLTR], F5 comprising SEQ ID NO: 20 [QSGNLAR], and F6 comprising SEQ ID NO: 21 [QSTPRTT].

47. The ZFN of any one of claims 36, 37, 39-41, 45, and 46, which comprises a ZFN pair comprising a first zinc finger DNA-binding domain and a second zinc finger DNA-binding domain, wherein the first DNA-binding domain comprises five zinc fingers, which comprises finger 1 (F1) comprising SEQ ID NO: 10 [RPYTLRL], finger 2 (F2) comprising SEQ ID NO: 11 [RSANLTR], finger 3 (F3) comprising SEQ ID NO: 12 [RSDALST], finger 4 (F4) comprising 13 [DRSTRTK], and finger 5 (F5) comprising SEQ ID NO: 14 [DRSTRTK], and wherein the second DNA-binding domain comprises six zinc fingers, which comprises F1 comprising SEQ ID NO: 16 [RSDVLSA], F2 comprising SEQ ID NO: 17 [DRSNRIK], F3 comprising SEQ ID NO: 18 [DRSHLTR], F4 comprising SEQ ID NO: 19 [LKQHLTR], F5 comprising SEQ ID NO: 20 [QSGNLAR], and F6 comprising SEQ ID NO: 21 [QSTPRTT].

48. The ZFN of any one of claims 36, 38, 42 and 43, wherein the DNA-binding domain comprises six zinc fingers, which comprises F1 comprising SEQ ID NO: 23 [RSDHLSR], F2 comprising SEQ ID NO: 24 [DSSDRKK], F3 comprising SEQ ID NO: 25 [RSDTLSE], F4 comprising 26 [QSGDLTR], and F5 comprising SEQ ID NO: 27 [QSSDLSR], and F6 comprising SEQ ID NO: 28 [YKWTLRN].

49. The ZFN of any one of claims 36, 38, 42, and 44, wherein the DNA-binding domain comprises five zinc fingers, which comprises F1 comprising SEQ ID NO: 30 [SNQNLTT], F2 comprising SEQ ID NO: 31 [DRSHLAR], F3 comprising SEQ ID NO: 32 [QSGDLTR], F4 comprising SEQ ID NO: 33 [WKHDLTN], and F5 comprising SEQ ID NO: 34 [TSGNLTR].

50. The ZFN of any one of claims 36, 38, 42-44, 48 and 49, which comprises a ZFN pair comprising a first zinc finger DNA-binding domain and a second zinc finger DNA-binding domain, wherein the first DNA-binding domain comprises six zinc fingers, which comprises F1 comprising SEQ ID NO: 23 [RSDHLSR], F2 comprising SEQ ID NO: 24 [DSSDRKK], F3 comprising SEQ ID NO: 25 [RSDTLSE], F4 comprising SEQ ID NO: 26 [QSGDLTR], and F5 comprising SEQ ID NO: 27 [QSSDLSR], and F6 comprising SEQ ID NO: 28 [YKWTLRN], and wherein the second DNA-binding domain comprises five zinc fingers, which comprises F1 comprising SEQ ID NO: 30 [SNQNLTT], F2 comprising SEQ ID NO: 31 [DRSHLAR], F3 comprising SEQ ID NO: 32 [QSGDLTR], F4 comprising SEQ ID NO: 33 [WKHDLTN], and F5 comprising SEQ ID NO: 34 [TSGNLTR].

51. The ZFN of any one of claim 36, 38, or 42-44, wherein the DNA-binding domain comprises SEQ ID NO: 54.

52. The ZFN of any one of claim 36, 38, or 42-44, wherein the DNA-binding domain comprises SEQ ID NO: 56.

53. The ZFN of any one of claim 36, 38, 42-44, or 51-52, wherein the ZEN pair comprises a first zinc finger DNA-binding domain and a second zinc finger DNA-binding domain, wherein the first DNA-binding domain comprises SEQ ID NO: 54 and the second DNA-binding domain comprises SEQ ID NO: 56.

54. The ZFN of any one of claims 36-53, wherein the cleavage domain comprises a FokI cleavage domain.

55. The ZFN of any one of claim 54, wherein the FokI cleavage domain further comprises one of more mutations at positions 418, 432, 441, 448, 476, 479, 481, 483, 486, 487, 490, 496, 499, 523, 525, 527, 537, 538 and 559 of SEQ ID NO 35.

56. The ZFN of claim 55, wherein the one or more mutations are at positions 479, 486, 496, 499, and/or 525.

57. The ZFN of claim 56, wherein the FokI cleavage domain comprises SEQ ID NO: 36 (FokELD).

58. The ZFN of claim 55, wherein the one or more mutations are at positions 490, 537, and/or 538.

59. The ZFN of claim 58, wherein the FokI cleavage domain comprises SEQ ID NO: 37 (FokKKR).

60. The ZFN of any one of claims 36 to 59, wherein the FokI cleavage domain forms a dimer prior to DNA cleavage.

61. The ZFN of claim 60, wherein the FokI dimer comprises a heterodimer.

62. The ZFN of claim 61, wherein the FokI heterodimer comprises a FokELD dimer and a FokKKR dimer.

63. The ZFN of any one of claims 36, 37, 38, 40, 45, and 47, wherein the ZFN comprises the amino acid sequence having at least about 70%, at least about 80%, at least about 85%, at least about 90%, at least about 95%, at least about 96%, at least about 97%, at least about 98%, at least about 99%, or about 100% sequence identity to SEQ ID NO: 5
(MDYKDHDGDYKDHDIDYKDDDDKMAPKKKRKVGIHGVPAAMGQLVKSEL
EEKKSELRHKLKYVPHEYIELIEIARNSTQDRILEMKVMEFFMKVYGYRG
KHLGGSRKPDGAIYTVGSPIDYGVIVDTKAYSGGYNLPIGQADEMERYVE
ENQTRDKHLNPNEWWKVYPSSVTEFKFLFVSGHFKGNYKAQLTRLNHITN
CNGAVLSVEELLIGGEMIKAGTLTLEEVRRKFNNGEINFSGAQGSTLDFR
PFQCRICMRNFSRPYTLRLHIRTHTGEKPFACDICGRKFARSANLTRHTK
IHTGSQKPFQCRICMRNFSRSDALSTHIRTHTGEKPFACDICGRKFADRS
TRTKHTKIHTGEKPFQCRICMRKFADRSTRTKHTKIHLRQKD)

64. The ZFN of any one of claims 36, 37, 39, 40, 46, and 47, wherein the ZFN comprises the amino acid sequence having at least about 70%, at least about 80%, at least about 85%, at least about 90%, at least about 95%, at least about 96%, at least about 97%, at least about 98%, at least about 99%, or about 100% sequence identity to SEQ ID NO: 6.
(MDYKDHDGDYKDHDIDYKDDDDKMAPKKKRKVGIHGVPAAMAERPFQCR
ICMQNFSRSDVLSAHIRTHTGEKPFACDICGKKFADRSNRIKHTKIHTGS
QKPFQCRICMQNFSDRSHLTRHIRTHTGEKPFACDICGRKFALKQHLTRH
TKIHTGEKPFQCRICMQNFSQSGNLARHIRTHTGEKPFACDICGRKFAQS
TPRTTHTKIHLRGSQLVKSELEEKKSELRHKLKYVPHEYIELIEIARNST
QDRILEMKVMEFFMKVYGYRGKHLGGSRKPDGAIYTVGSPIDYGVIVDTK
AYSGGYNLPIGQADEMQRYVKENQTRNKHINPNEWWKVYPSSVTEFKFLF
VSGHFKGNYKAQLTRLNRKTNCNGAVLSVEELLIGGEMIKAGTLTLEEVR
RKFNNGEINF)

65. The ZFN of any one of claims 36, 38, 42, 43, 48, and 53, wherein the ZFN comprises the amino acid sequence having at least about 70%, at least about 80%, at least about 85%, at least about 90%, at least about 95%, at least about 96%, at least about 97%, at least about 98%, at least about 99%, or about 100% sequence identity to SEQ ID NO: 7 (MDYKDHDGDYKDHDIDYKDDDDKMAPKKKRKVGIHGVPAAMGQLVKSELEEKKSELRHKLKYVPHEYIELIEIARNS TQDRILEMKVMEFFMKVYGYRGKHLGGSRKPDGAIYTVGSPIDYGVIVDTKAYSGGYNLPIGQADEMERYVEENQTRD KHLNPNEWWKVYPSSVTEFKFLFVSGHFKGNYKAQLTRINHITNCNGAVLSVEELLIGGEMIKAGTLTLEEVRRKENN GEINFSGTPHEVGVYTLRPFQCRICMRNFSRSDHLSRHIRTHTGEKPFACDICGRKFADSSDRKKHTKIHTGEKPFQC RICMRNFSRSDTLSEHIRTHTGEKPFACDICGRKFAQSGDLTRHTKIHTHPRAPIPKPFQCRICMRNFSQSSDLSRHI RTHTGEKPFACDICGRKFAYKWTLRNHTKIHLRQKD) or SEQ ID NO: 54 (MDYKDHDGDYKDHDIDYKDDDDKMAPKKKRKVGIHGVPAAMGQLVKSELEEKKSELRHKLKYVPHEYIELIEIARNS TQDRILEMKVMEFFMKVYGYRGKHLGGSRKPDGAIYTVGSPIDYGVIVDTKAYSGGYNLPIGQADEMERYVEENQTRD KHLNPNEWWKVYPSSVTEFKFLFVSGHFSGNYKAQLTRLNHITNCNGAVLSVEELLIGGEMIKAGTLTLEEVRRKENN GEINFSGTPHEVGVYTLRPFQCRICMRNFSRSDHLSRHIRTHTGEKPFACDICGRKFADSSDRKKHTKIHTGEKPFQC RICMRNFSRSDTLSEHIRTHTGEKPFACDICGRKFAQSGDLTRHTKIHTHPRAPIPKPFQCRICMRNFSQSSDLSRHI RTHTGEKPFACDICGRKFAYKWTLRNHTKIHLRQKD).

66. The ZFN of any one of claims 36, 38, 42, 43, 49 and 53, wherein the ZFN comprises the amino acid sequence having at least about 70%, at least about 80%, at least about 85%, at least about 90%, at least about 95%, at least about 96%, at least about 97%, at least about 98%, at least about 99%, or about 100% sequence identity to SEQ ID NO: 8 (MDYKDHDGDYKDHDIDYKDDDDKMAPKKKRKVGIHGVPAAMGQLVKSELEEKKSELRHKLKYVPHEYIELIEIARNS TQDRILEMKVMEFFMKVYGYRGKHLGGSRKPDGAIYTVGSPIDYGVIVDTKAYSGGYNLPIGQADEMQRYVKENQTRN KHINPNEWWKVYPSSVTEFKFLFVSGHFKGNYKAQLTRLNRKTNCNGAVLSVEELLIGGEMIKAGTLTLEEVRRKENN GEINFSGTPHEVGVYTLRPFQCRICMRNFSSNQNLTTHIRTHTGEKPFACDICGRKFADRSHLARHTKIHTGEKPFQC RICMQKFAQSGDLTRHTKIHTGEKPFQCRICMQNFSWKHDLTNHIRTHTGEKPFACDICGRKFATSGNLTRHTKIHLR QKD) or SEQ ID NO: 56 (QLVKSELEEKKSELRHKLKYVPHEYIELIEIARNSTQDRILEMKVMEFFMKVYGYRGKHLGGSRKPDGAIYTVGSPI DYGVIVDTKAYSGGYNLPTGQADEMQRYVKENQTRNKHINPNEWWKVYPSSVTEFKFLFVSGHFKGNYKAQLTRLNRK TNCNGAVLSVEELLIGGEMIKAGTLTLEEVRRKFNNGEINFSGTPHEVGVYTLRPFQCRICMRNFSSNQNLTTHIRTH TGEKPFACDICGRKFADRSHLARHTKIHTGEKPFQCRICMQKFAQSGDLTRHTKIHTGEKPFQCRICMQNFSWKHDLT NHIRTHTGEKPFACDICGRKFATSGNLTRHTKIHLROKD).

67. The ZFN of any one of claims 36, 38, 42 and 43, wherein the DNA-binding domain comprises six zinc fingers, which comprises F1 comprising SEQ ID NO: 23 [RSDHLSR], F2 comprising SEQ ID NO: 24 [DSSDRKK], F3 comprising SEQ ID NO: 25 [RSDTLSE], F4 comprising 26 [QSGDLTR], and F5 comprising SEQ ID NO: 27 [QSSDLSR], and F6 comprising SEQ ID NO: 28 [YKWTLRN], wherein the cleavage domain comprises a FokI cleavage domain, and wherein the FokI cleavage domain further comprises K to S mutation at position 525 of SEQ ID NO 35.

68. The ZFN of any one of claims 36, 38, 42, and 44, wherein the DNA-binding domain comprises five zinc fingers, which comprises F1 comprising SEQ ID NO: 30 [SNQNLTT], F2 comprising SEQ ID NO: 31 [DRSHLAR], F3 comprising SEQ ID NO: 32 [QSGDLTR], F4 comprising SEQ ID NO: 33 [WKHDLTN], and F5 comprising SEQ ID NO: 34 [TSGNLTR], wherein the cleavage domain comprises a FokI cleavage domain, and wherein the FokI cleavage domain further comprises I to T mutation at position 479 of SEQ ID NO 35.

69. The ZFN of any one of claims 36, 38, 42-44, 48 and 49, which comprises a ZFN pair comprising a first zinc finger DNA-binding domain, a first cleavage domain, a second zinc finger DNA-binding domain, and a second cleavage domain, wherein the first DNA-binding domain comprises six zinc fingers, which comprises F1 comprising SEQ ID NO: 23 [RSDHLSR], F2 comprising SEQ ID NO: 24 [DSSDRKK], F3 comprising SEQ ID NO: 25 [RSDTLSE], F4 comprising SEQ ID NO: 26 [QSGDLTR], and F5 comprising SEQ ID NO: 27 [QSSDLSR], and F6 comprising SEQ ID NO: 28 [YKWTLRN], and wherein the second DNA-binding domain comprises five zinc fingers, which comprises F1 comprising SEQ ID NO: 30 [SNQNLTT], F2 comprising SEQ ID NO: 31 [DRSHLAR], F3 comprising SEQ ID NO: 32 [QSGDLTR], F4 comprising SEQ ID NO: 33 [WKHDLTN], and F5 comprising SEQ ID NO: 34 [TSGNLTR], wherein the first and the second cleavage domain comprise a FokI cleavage domain, and wherein the first FokI cleavage domain further comprises K to S mutation at position 525 of SEQ ID NO 35 and the second FokI cleavage domain further comprises I to T mutation at position 479 of SEQ ID NO 35.

70. A ZFN pair comprising a first ZFN and a second ZFN, wherein the first ZFN comprises the amino acid sequence having at least about 70%, at least about 80%, at least about 85%, at least about 90%, at least about 95%, at least about 96%, at least about 97%, at least about 98%, at least about 99%, or about 100% sequence identity to SEQ ID NO: 5. (MDYKDHDGDYKDHDIDYKDDDDKMAPKKKRKVGIHGVPAAMGOLVKSELEEKKSELRHKLKYVPHEYIELIEIARNS TQDRILEMKVMEFFMKVYGYRGKHLGGSRKPDGAIYTVGSPIDYGVIVDTKAYSGGYNLPIGQADEMERYVEENQTRD KHLNPNEWWKVYPSSVTEFKFLFVSGHFKGNYKAQLTRINHITNCNGAVLSVEELLIGGEMIKAGTLTLEEVRRKENN GEINFSGAQGSTLDFRPFQCRICMRNFSRPYTLRLHIRTHTGEKPFACDICGRKFARSANLTRHTKIHTGSQKPFQCR ICMRNFSRSDALSTHIRTHTGEKPFACDICGRKFADRSTRTKHTKIHTGEKPFQCRICMRKFADRSTRTKHTKIHLRQ KD) and the second ZFN comprises the amino acid sequence having at least about 70%, at least about 80%, at least about 85%, at least about 90%, at least about 95%, at least about 96%, at least about 97%, at least about 98%, at least about 99%, or about 100% sequence identity to SEQ ID NO: 6.
(MDYKDHDGDYKDHDIDYKDDDDKMAPKKKRKVGIHGVPAAMAERPFQCR
ICMQNFSRSDVLSAHIRTHTGEKPFACDICGKKFADRSNRIKHTKIHTGS
QKPFQCRICMQNFSDRSHLTRHIRTHTGEKPFACDICGRKFALKQHLTRH
TKIHTGEKPFQCRICMQNFSQSGNLARHIRTHTGEKPFACDICGRKFAQS
TPRTTHTKIHLRGSQLVKSELEEKKSELRHKLKYVPHEYIELIEIARNST
QDRILEMKVMEFFMKVYGYRGKHLGGSRKPDGAIYTVGSPIDYGVIVDTK
AYSGGYNLPIGQADEMQRYVKENQTRNKHINPNEWWKVYPSSVTEFKFLF
VSGHFKGNYKAQLTRLNRKTNCNGAVLSVEELLIGGEMIKAGTLTLEEVR
RKFNNGEINF).

71. A ZFN pair comprising a first ZFN and a second ZFN, wherein the first ZFN comprises the amino acid sequence having at least about 70%, at least about 80%, at least about 85%, at least about 90%, at least about 95%, at least about 96%, at least about 97%, at least about 98%, at least about 99%, or about 100% sequence identity to SEQ ID NO: 7 (MDYKDHDGDYKDHDIDYKDDDDKMAPKKKRKVGIHGVPAAMGQLVKSELEEKKSELRHKLKYVPHEYIELIEIARNS TQDRILEMKVMEFFMKVYGYRGKHLGGSRKPDGAIYTVGSPIDYGVIVDTKAYSGGYNLPIGQADEMERYVEENQTRD KHLNPNEWWKVYPSSVTEFKFLFVSGHFKGNYKAQLTRLNHITNCNGAVLSVEELLIGGEMIKAGTLTLEEVRRKENN GEINFSGTPHEVGVYTLRPFQCRICMRNFSRSDHLSRHIRTHTGEKPFACDICGRKFADSSDRKKHTKIHTGEKPFQC RICMRNFSRSDTLSEHIRTHTGEKPFACDICGRKFAQSGDLTRHTKIHTHPRAPIPKPFQCRICMRNFSQSSDLSRHI RTHTGEKPFACDICGRKFAYKWTLRNHTKIHLROKD) and the second ZFN comprises the amino acid sequence having at least about 70%, at least about 80%, at least about 85%, at least about 90%, at least about 95%, at least about 96%, at least about 97%, at least about 98%, at least about 99%, or about 100% sequence identity to SEQ ID NO: 8
(MDYKDHDGDYKDHDIDYKDDDDKMAPKKKRKVGIHGVPAAMGQLVKSEL
EEKKSELRHKLKYVPHEYIELIEIARNSTQDRILEMKVMEFFMKVYGYRG
KHLGGSRKPDGAIYTVGSPIDYGVIVDTKAYSGGYNLPIGQADEMQRYVK
ENQTRNKHINPNEWWKVYPSSVTEFKFLFVSGHFKGNYKAQLTRLNRKTN
CNGAVLSVEELLIGGEMIKAGTLTLEEVRRKFNNGEINFSGTPHEVGVYT
LRPFQCRICMRNFSSNQNLTTHIRTHTGEKPFACDICGRKFADRSHLARH
TKIHTGEKPFQCRICMQKFAQSGDLTRHTKIHTGEKPFQCRICMQNFSWK
HDLTNHIRTHTGEKPFACDICGRKFATSGNLTRHTKIHLRQKD).

72. A ZFN pair comprising a first ZFN and a second ZFN, wherein the first ZFN comprises the amino acid sequence having at least about 70%, at least about 80%, at least about 85%, at least about 90%, at least about 95%, at least about 96%, at least about 97%, at least about 98%, at least about 99%, or about 100% sequence identity to SEQ ID NO: 54 and the second ZFN comprises the amino acid sequence having at least about 70%, at least about 80%, at least about 85%, at least about 90%, at least about 95%, at least about 96%, at least about 97%, at least about 98%, at least about 99%, or about 100% sequence identity to SEQ ID NO: 56.

73. A ZFN encoded by the polynucleotide of any one of claims 1 to 35.

74. A polynucleotide encoding the ZFN of any one of claims 36 to 60 or the ZFN pair of any one of claims 70-72.

75. An isolated cell comprising the polynucleotides of claims 1 to 35 and 74, the ZFNs according to claims 36 to 69 and 73, or the ZFN pair of any one of claims 70-72.

76. The isolated cell of claim 75, wherein the isolated cell comprises a T cell, a NK cell, a tumor infiltrating lymphocyte, a stem cell, Mesenchymal stem cells (MSC), hematopoietic stem cells (HSC), fibroblasts, cardiomyocytes, pancreatic islet cells, or a blood cell.

77. The isolated cell of claim 754 or 76, which is allogeneic.

78. The isolated cell of claim 75 or 76, which is autologous.

79. A method of preparing a T cell, comprising contacting an isolated T cell with the polynucleotides of claims 1 to 35 and 74, the ZFNs according to claims 36 to 69 and 73, or the ZFN pair of any one of claims 70-72.

80. The method of claim 79, wherein the T cell comprises a chimeric antigen receptor T cell, a T cell receptor cell, a Treg cell, a Tumor infiltrating lymphocyte, or any combination thereof.

81. A method of treating a subject in need of a cellular therapy comprising administering the isolated cell of any one of claims 75-78 to the subject.

82. The method of claim 81, wherein the isolated cells are allogeneic or autologous.


SEQUENCE LISTING (57 sequences)

SEQ ID NO: 1 - protein, 1130 aa, Artificial Sequence
  CIITA Isoform 1 (UniProt identifier P33076-1)
MRCLAPRPAGSYLSEPQGSSQCATMELGPLEGGYLELLNSDADPLCLYHFYDQMDLAGEEEIELYSEPDTDTINCDQFSRLLCDMEGDEETREAYANIAELDQYVFQDSQLEGLSKDIFKHIGPDEVIGESMEMPAEVGQKSQKRPFPEELPADLKHWKPAEPPTVVTGSLLVRPVSDCSTLPCLPLPALFNQEPASGQMRLEKTDQIPMPFSSSSLSCLNLPEGPIQFVPTISTLPHGLWQISEAGTGVSSIFIYHGEVPQASQVPPPSGFTVHGLPTSPDRPGSTSPFAPSATDLPSMPEPALTSRANMTEHKTSPTQCPAAGEVSNKLPKWPEPVEQFYRSLQDTYGAEPAGPDGILVEVDLVQARLERSSSKSLERELATPDWAERQLAQGGLAEVLLAAKEHRRPRETRVIAVLGKAGQGKSYWAGAVSRAWACGRLPQYDFVFSVPCHCLNRPGDAYGLQDLLFSLGPQPLVAADEVFSHILKRPDRVLLILDGFEELEAQDGFLHSTCGPAPAEPCSLRGLLAGLFQKKLLRGCTLLLTARPRGRLVQSLSKADALFELSGFSMEQAQAYVMRYFESSGMTEHQDRALTLLRDRPLLLSHSHSPTLCRAVCQLSEALLELGEDAKLPSTLTGLYVGLLGRAALDSPPGALAELAKLAWELGRRHQSTLQEDQFPSADVRTWAMAKGLVQHPPRAAESELAFPSFLLQCFLGALWLALSGEIKDKELPQYLALTPRKKRPYDNWLEGVPRFLAGLIFQPPARCLGALLGPSAAASVDRKQKVLARYLKRLQPGTLRARQLLELLHCAHEAEEAGIWQHVVQELPGRLSFLGTRLTPPDAHVLGKALEAAGQDFSLDLRSTGICPSGLGSLVGLSCVTRFRAALSDTVALWESLQQHGETKLLQAAEEKFTIEPFKAKSLKDVEDLGKLVQTQRTRSSSEDTAGELPAVRDLKKLEFALGPVSGPQAFPKLVRILTAFSSLQHLDLDALSENKIGDEGVSQLSATFPQLKSLETLNLSQNNITDLGAYKLAEALPSLAASLLRLSLYNNCICDVGAESLARVLPDMVSLRVMDVQYNKFTAAGAQQLAASLRRCPHVETLAMWTPTIPFSVQEHLQQQDSRISLR

SEQ ID NO: 2 - protein, 883 aa, artificial sequence
  CIITA Isoform 2 (identifier P33076-2)
MRCLAPRPAGSYLSEPQGSSQCATMELGPLEGGYLELLNSDADPLCLYHFYDQMDLAGEEEIELYSEPDTDTINCDQFSRLLCDMEGDEETREAYANIAELDQYVFQDSQLEGLSKDIFKHIGPDEVIGESMEMPAEVGQKSQKRPFPEELPADLKHWKPVPFSSSSLSCLNLPEGPIQFVPTISTLPHGLWQISEAGTGVSSIFIYHGEVPQASQVPPPSGFTVHGLPTSPDRPGSTSPFAPSATDLPSMPEPALTSRANMTEHKTSPTQCPAAGEVSNKLPKWPEPVEQFYRSLQDTYGAEPAGPDGILVEVDLVQARLERSSSKSLERELATPDWAERQLAQGGLAEVLLAAKEHRRPRETRVIAVLGKAGQGKSYWAGAVSRAWACGRLPQYDFVFSVPCHCLNRPGDAYGLQDLLFSLGPQPLVAADEVFSHILKRPDRVLLILDGFEELEAQDGFLHSTCGPAPAEPCSLRGLLAGLFQKKLLRGCTLLLTARPRGRLVQSLSKADALFELSGFSMEQAQAYVMRYFESSGMTEHQDRALTLLRDRPLLLSHSHSPTLCRAVCQLSEALLELGEDAKLPSTLTGLYVGLLGRAALDSPPGALAELAKLAWELGRRHQSTLQEDQFPSADVRTWAMAKGLVQHPPRAAESELAFPSFLLQCFLGALWLALSGEIKDKELPQYLALTPRKKRPYDNWLEGVPRFLAGLIFQPPARCLGALLGPSAAASVDRKQKVLARYLKRLQPGTLRARQLLELLHCAHEAEEAGIWQHVVQELPGRLSFLGTRLTPPDAHVLGKALEAAGQDFSLDLRSTGICPSGLGSLVGLSCVTRFRWGEGLGRDILVLGINCGLGAKPSALWGPFSMQSSRVGQNGFSPFLR

SEQ ID NO: 3 - protein, 545 aa, artificial sequence
  “CIITA Isoform 3 (identifier P33076-3) ”
MRCLAPRPAGSYLSEPQGSSQCATMELGPLEGGYLELLNSDADPLCLYHFYDQMDLAGEEEIELYSEPDTDTINCDQFSRLLCDMEGDEETREAYANIAELDQYVFQDSQLEGLSKDIFKHIGPDEVIGESMEMPAEVGQKSQKRPFPEELPADLKHWKPVPFSSSSLSCLNLPEGPIQFVPTISTLPHGLWQISEAGTGVSSIFIYHGEVPQASQVPPPSGFTVHGLPTSPDRPGSTSPFAPSATDLPSMPEPALTSRANMTEHKTSPTQCPAAGEVSNKLPKWPGLAWSPCLGLRPSLHRAALSDTVALWESLQQHGETKLLQAAEEKFTIEPFKAKSLKDVEDLGKLVQTQRTRSSSEDTAGELPAVRDLKKLEFAGPVSGPQAFPKLVRILTAFSSLQHLDLDALSENKIGDEGVSQLSATFPQLKSLETLNLSQNNITDLGAYKLAEALPSLAASLLRLSLYNNCICDVGAESLARVLPDMVSLRVMDVQYNKFTAAGAQQLAASLRRCPHVETLAMWTPTIPFSVQEHLQQQDSRISLR

SEQ ID NO: 4 - protein, 932 aa, artificial sequence
  CIITA Isoform 4 (identifier P33076-4)
MRCLAPRPAGSYLSEPQGSSQCATMELGPLEGGYLELLNSDADPLCLYHFYDQMDLAGEEEIELYSEPDTDTINCDQFSRLLCDMEGDEETREAYANIAELDQYVFQDSQLEGLSKDIFKHIGPDEVIGESMEMPAEVGQKSQKRPFPEELPADLKHWKPAEPPTVVTGSLLVRPVSDCSTLPCLPLPALFNQEPASGQMRLEKTDQIPMPFSSSSLSCLNLPEGPIQFVPTISTLPHGLWQISEAGTGVSSIFIYHGEVPQASQVPPPSGFTVHGLPTSPDRPGSTSPFAPSATDLPSMPEPALTSRANMTEHKTSPTQCPAAGEVSNKLPKWPEPVEQFYRSLQDTYGAEPAGPDGILVEVDLVQARLERSSSKSLERELATPDWAERQLAQGGLAEVLLAAKEHRRPRETRVIAVLGKAGQGKSYWAGAVSRAWACGRLPQYDFVFSVPCHCLNRPGDAYGLQDLLFSLGPQPLVAADEVFSHILKRPDRVLLILDGFEELEAQDGFLHSTCGPAPAEPCSLRGLLAGLFQKKLLRGCTLLLTARPRGRLVQSLSKADALFELSGFSMEQAQAYVMRYFESSGMTEHQDRALTLLRDRPLLLSHSHSPTLCRAVCQLSEALLELGEDAKLPSTLTGLYVGLLGRAALDSPPGALAELAKLAWELGRRHQSTLQEDQFPSADVRTWAMAKGLVQHPPRAAESELAFPSFLLQCFLGALWLALSGEIKDKELPQYLALTPRKKRPYDNWLEGVPRFLAGLIFQPPARCLGALLGPSAAASVDRKQKVLARYLKRLQPGTLRARQLLELLHCAHEAEEAGIWQHVVQELPGRLSFLGTRLTPPDAHVLGKALEAAGQDFSLDLRSTGICPSGLGSLVGLSCVTRFRWGEGLGRDILVLGINCGLGAKPSALWGPFSMQSSRVGQNGFSPFLR

SEQ ID NO: 5 - protein, 391 aa, artificial sequence
  Zinc finger nuclease 76867
MDYKDHDGDYKDHDIDYKDDDDKMAPKKKRKVGIHGVPAAMGQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRDKHLNPNEWWKVYPSSVTEFKFLFVSGHFKGNYKAQLTRLNHITNCNGAVLSVEELLIGGEMIKAGTLTLEEVRRKFNNGEINFSGAQGSTLDFRPFQCRICMRNFSRPYTLRLHIRTHTGEKPFACDICGRKFARSANLTRHTKIHTGSQKPFQCRICMRNFSRSDALSTHIRTHTGEKPFACDICGRKFADRSTRTKHTKIHTGEKPFQCRICMRKFADRSTRTKHTKIHLRQKD

SEQ ID NO: 6 - protein, 409 aa, artificial sequence
  zinc finger nuclease 82862
MDYKDHDGDYKDHDIDYKDDDDKMAPKKKRKVGIHGVPAAMAERPFQCRICMQNFSRSDVLSAHIRTHTGEKPFACDICGKKFADRSNRIKHTKIHTGSQKPFQCRICMQNFSDRSHLTRHIRTHTGEKPFACDICGRKFALKQHLTRHTKIHTGEKPFQCRICMQNFSQSGNLARHIRTHTGEKPFACDICGRKFAQSTPRTTHTKIHLRGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINF

SEQ ID NO: 7 - protein, 425 aa, artificial sequence
  zinc finger nuclease 87254
MDYKDHDGDYKDHDIDYKDDDDKMAPKKKRKVGIHGVPAAMGQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRDKHLNPNEWWKVYPSSVTEFKFLFVSGHFKGNYKAQLTRLNHITNCNGAVLSVEELLIGGEMIKAGTLTLEEVRRKFNNGEINFSGTPHEVGVYTLRPFQCRICMRNFSRSDHLSRHIRTHTGEKPFACDICGRKFADSSDRKKHTKIHTGEKPFQCRICMRNFSRSDTLSEHIRTHTGEKPFACDICGRKFAQSGDLTRHTKIHTHPRAPIPKPFQCRICMRNFSQSSDLSRHIRTHTGEKPFACDICGRKFAYKWTLRNHTKIHLRQKD

SEQ ID NO: 8 - protein, 392 aa, artificial sequence
  zinc finger nuclease 84221
MDYKDHDGDYKDHDIDYKDDDDKMAPKKKRKVGIHGVPAAMGQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINFSGTPHEVGVYTLRPFQCRICMRNFSSNQNLTTHIRTHTGEKPFACDICGRKFADRSHLARHTKIHTGEKPFQCRICMQKFAQSGDLTRHTKIHTGEKPFQCRICMQNFSWKHDLTNHIRTHTGEKPFACDICGRKFATSGNLTRHTKIHLRQKD

SEQ ID NO: 9 - DNA, 15 bp, artificial sequence
  dna binding sequence of ZFP 76867
gccaccatggagttg

SEQ ID NO: 10 - protein, 7 aa, artificial sequence
  ZFP 76867 Finger 1
RPYTLRL

SEQ ID NO: 11 - protein, 7 aa, artificial sequence
  ZFP 76867 Finger 2
RSANLTR

SEQ ID NO: 12 - protein, 7 aa, artificial sequence
  ZFP 76867 Finger 3
RSDALST

SEQ ID NO: 13 - protein, 7 aa, artificial sequence
  ZFP 76867 Finger 4
DRSTRTK

SEQ ID NO: 14 - protein, 7 aa, artificial sequence
  ZFP 76867 Finger 5
DRSTRTK

SEQ ID NO: 15 - DNA, 18 bp, artificial sequence
  dna binding sequence of ZFP 82862
ctagaaggtggctacctg

SEQ ID NO: 16 - protein, 7 aa, artificial sequence
  ZFP 82862 Finger 1
RSDVLSA

SEQ ID NO: 17 - protein, 7 aa, artificial sequence
  ZFP 82862 Finger 2
DRSNRIK

SEQ ID NO: 18 - protein, 7 aa, artificial sequence
  ZFP 82862 Finger 3
DRSHLTR

SEQ ID NO: 19 - protein, 7 aa, artificial sequence
  ZFP 82862 Finger 4
LKQHLTR

SEQ ID NO: 20 - protein, 7 aa, artificial sequence
  ZFP 82862 Finger 5
QSGNLAR

SEQ ID NO: 21 - protein, 7 aa, artificial sequence
  ZFP 82862 Finger 6
QSTPRTT

SEQ ID NO: 22 - DNA, 12 bp, artificial sequence
  dna binding sequence of ZFP 87254
gaaccgtccggg

SEQ ID NO: 23 - protein, 7 aa, artificial sequence
  ZFP 87254 Finger 1
RSDHLSR

SEQ ID NO: 24 - protein, 7 aa, artificial sequence
  ZFP 87254 Finger 2
DSSDRKK

SEQ ID NO: 25 - protein, 7 aa, artificial sequence
  ZFP 87254 Finger 3
RSDTLSE

SEQ ID NO: 26 - protein, 7 aa, artificial sequence
  ZFP 87254 Finger 4
QSGDLTR

SEQ ID NO: 27 - protein, 7 aa, artificial sequence
  ZFP 87254 Finger 5
QSSDLSR

SEQ ID NO: 28 - protein, 7 aa, artificial sequence
  ZFP 87254 Finger 6
YKWTLRN

SEQ ID NO: 29 - DNA, 15 bp, artificial sequence
  dna binding sequence of ZFP 84221
gatcctgcaggccat

SEQ ID NO: 30 - protein, 7 aa, artificial sequence
  ZFP 84221 Finger 1
SNQNLTT

SEQ ID NO: 31 - protein, 7 aa, artificial sequence
  ZFP 84221 Finger 2
DRSHLAR

SEQ ID NO: 32 - protein, 7 aa, artificial sequence
  ZFP 84221 Finger 3
QSGDLTR

SEQ ID NO: 33 - protein, 7 aa, artificial sequence
  ZFP 84221 Finger 4
WKHDLTN

SEQ ID NO: 34 - protein, 7 aa, artificial sequence
  ZFP 84221 Finger 5
TSGNLTR

SEQ ID NO: 35 - protein, 579 aa, artificial sequence
  FokI protein
MVSKIRTFGWVQNPGKFENLKRVVQVFDRNSKVHNEVKNIKIPTLVKESKIQKELVAIMNQHDLIYTYKELVGTGTSIRSEAPCDAIIQATIADQGNKKGYIDNWSSDGFLRWAHALGFIEYINKSDSFVITDVGLAYSKSADGSAIEKEILIEAISSYPPAIRILTLLEDGQHLTKFDLGKNLGFSGESGFTSLPEGILLDTLANAMPKDKGEIRNNWEGSSDKYARMIGGWLDKLGLVKQGKKEFIIPTLGKPDNKEFISHAFKITGEGLKVLRRAKGSTKFTRVPKRVYWEMLATNLTDKEYVRTRRALILEILIKAGSLKIEQIQDNLKKLGFDEVIETIENDIKGLINTGIFIEIKGRFYQLKDHILQFVIPNRGVTKQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 36 - protein, 196 aa, artificial sequence
  FokIELD protein
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRDKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 37 - protein, 196 aa, artificial sequence
  FokIKKR protein
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINF

SEQ ID NO: 38 - DNA, 19 bp, artificial sequence
  dna binding sequence of ZFP 87254
attgcttgaaccgtccggg

SEQ ID NO: 39 - DNA, 5620 bp, artificial sequence
  STV220-pVAX-GEM2UX-CIITA-B-76867-2A-82862 plasmid
gctgcttcgcgatgtacgggccagatatacgcgcatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctaatacgactcactatagggagacaagcttgaatacaagcttgcttgttctttttgcagaagctcagaataaacgctcaactttggcagatcgaattcgccatggactacaaagaccatgacggtgattataaagatcatgacatcgattacaaggatgacgatgacaagatggcccccaagaagaagaggaaggtcggcatccacggggtacccgccgctatgggacagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcaatggcgccgtgctgagcgtggaggagctgctgatcggcggcgagatgatcaaagccggcaccctgacactggaggaggtgcggcgcaagttcaacaacggcgagatcaacttcagcggcgctcagggatctaccctggactttaggcccttccagtgtcgaatctgcatgcgtaacttcagtcgcccgtacaccctgcgcctgcacatccgcacccacaccggcgagaagccttttgcctgtgacatttgtgggaggaaatttgcccgctccgccaacctgacccgccataccaagatacacacgggcagccaaaagcccttccagtgtcgaatctgcatgcgtaacttcagtcgtagtgacgccctgagcacgcacatccgcacccacacaggcgagaagccttttgcctgtgacatttgtgggaggaaatttgccgacaggagcacccgcacaaagcataccaagatacacacgggcgagaagcccttccagtgtcgaatctgcatgcgtaagtttgccgaccgctccacccgcaccaagcataccaagatacacctgcggcagaaggacagatctggcggcggagagggcagaggaagtcttctaacctgcggtgacgtggaggagaatcccggccctaggaccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcagaacttcagtcgtagtgacgtcctgagcgcacacatccgcacccacacaggcgagaagccttttgcctgtgacatttgtgggaagaaatttgccgacaggagcaaccgcataaagcataccaagatacacacgggcagccaaaagcccttccagtgtcgaatctgcatgcagaacttcagtgaccgctcccacctgacccgccacatccgcacccacaccggcgagaagccttttgcctgtgacatttgtgggaggaaatttgccctgaagcagcacctgacccgccataccaagatacacacgggcgagaagcccttccagtgtcgaatctgcatgcagaacttcagtcagtccggcaacctggcccgccacatccgcacccacaccggcgagaagccttttgcctgtgacatttgtgggaggaaatttgcccagtccaccccgcgcaccacccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttctgataactcgagtctagaagctcgctttcttgctgtccaatttctattaaaggttcctttgttccctaagtccaactactaaactgggggatattatgaagggccttgagcatctggattctgcctaataaaaaacatttattttcattgctgcgctagaagctcgctttcttgctgtccaatttctattaaaggttcctttgttccctaagtccaactactaaactgggggatattatgaagggccttgagcatctggattctgcctaataaaaaacatttattttcattgctgcgggacattcttaattaaaaaaaaaaaaaaaaaaaaaaaaaaaaaaaaaaaaaaaaaaaaaaaaaaaaaaaaaaaactagtggcgcctgatgcggtattttctccttacgcatctgtgcggtatttcacaccgcataatccagcacagtggcggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 40 - DNA, 5671 bp, artificial sequence
  STV220-pVAX-GEM2UX-CIITA-G-87254-2A-84221 plasmid
gctgcttcgcgatgtacgggccagatatacgcgcatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctaatacgactcactatagggagacaagcttgaatacaagcttgcttgttctttttgcagaagctcagaataaacgctcaactttggcagatcgaattcgccatggactacaaagaccatgacggtgattataaagatcatgacatcgattacaaggatgacgatgacaagatggcccccaagaagaagaggaaggtcggcatccacggggtacccgccgctatgggacagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcaatggcgccgtgctgagcgtggaggagctgctgatcggcggcgagatgatcaaagccggcaccctgacactggaggaggtgcggcgcaagttcaacaacggcgagatcaacttcagcggcactccacacgaagtgggagtgtacacacttaggcccttccagtgtcgaatctgcatgcgtaacttcagtcgtagtgaccacctgagccggcacatccgcacccacacaggcgagaagccttttgcctgtgacatttgtgggaggaaatttgccgacagcagcgaccgcaaaaagcataccaagatacacacgggcgagaagcccttccagtgtcgaatctgcatgcgtaacttcagtcgctccgacaccctgtccgagcacatccgcacccacaccggcgagaagccttttgcctgtgacatttgtgggaggaaatttgcccagtccggcgacctgacccgccataccaagatacacacgcacccgcgcgccccgatcccgaagcccttccagtgtcgaatctgcatgcgtaacttcagtcagtcctccgacctgtcccgccacatccgcacccacaccggcgagaagccttttgcctgtgacatttgtgggaggaaatttgcctacaagtggaccctgcgcaaccataccaagatacacctgcggcagaaggacagatctggcggcggagagggcagaggaagtcttctaacctgcggtgacgtggaggagaatcccggccctaggaccatggactacaaagaccatgacggtgattataaagatcatgacatcgattacaaggatgacgatgacaagatggcccccaagaagaagaggaaggtcggcattcatggggtacccgccgctatgggacagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttcagcggcactccacacgaagtgggagtgtacacacttaggcccttccagtgtcgaatctgcatgcgtaacttcagttccaaccagaacctgaccacccacatccgcacccacaccggcgagaagccttttgcctgtgacatttgtgggaggaaatttgccgaccgctcccacctggcccgccataccaagatacacacgggcgagaagcccttccagtgtcgaatctgcatgcagaagtttgcccagtccggcgacctgacccgccataccaagatacacacgggcgagaagcccttccagtgtcgaatctgcatgcagaacttcagttggaagcacgacctgaccaaccacatccgcacccacaccggcgagaagccttttgcctgtgacatttgtgggaggaaatttgccacctccggcaacctgacccgccataccaagatacacctgcggcagaaggactgataactcgagtctagaagctcgctttcttgctgtccaatttctattaaaggttcctttgttccctaagtccaactactaaactgggggatattatgaagggccttgagcatctggattctgcctaataaaaaacatttattttcattgctgcgctagaagctcgctttcttgctgtccaatttctattaaaggttcctttgttccctaagtccaactactaaactgggggatattatgaagggccttgagcatctggattctgcctaataaaaaacatttattttcattgctgcgggacattcttaattaaaaaaaaaaaaaaaaaaaaaaaaaaaaaaaaaaaaaaaaaaaaaaaaaaaaaaaaaaaactagtggcgcctgatgcggtattttctccttacgcatctgtgcggtatttcacaccgcataatccagcacagtggcggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 41 - DNA, 24 bp, artificial sequence
  primer
gcagagctctctggctaactagag

SEQ ID NO: 42 - DNA, 80 bp, artificial sequence
  primer
ttttttttttttttttttttttttttttttttttttttttttttttttttttttttttttctggcaactagaaggcacag

SEQ ID NO: 43 - DNA, 47 bp, artificial sequence
  primer
  misc_feature (21)..(24): n is a, c, g, or t
acacgacgctcttccgatctnnnngttgtaggtgtcaattttctgcc

SEQ ID NO: 44 - DNA, 42 bp, artificial sequence
  primer
gacgtgtgctcttccgatctatctggtcatagaagtggtaga

SEQ ID NO: 45 - DNA, 46 bp, artificial sequence
  primer
  misc_feature (21)..(24): n is a, c, g, or t
acacgacgctcttccgatctnnnntccccagtacgactttgtcttc

SEQ ID NO: 46 - DNA, 42 bp, artificial sequence
  primer
gacgtgtgctcttccgatcttcaagatgtggctgaaaacctc

SEQ ID NO: 47 - DNA, 63 bp, artificial sequence
  primer
  misc_feature (30)..(37): n is a, c, g, or t
aatgatacggcgaccaccgagatctacacnnnnnnnnacactctttccctacacgacgctctt

SEQ ID NO: 48 - DNA, 74 bp, artificial sequence
  primer
  misc_feature (25)..(32): n is a, c, g, or t
caagcagaagacggcatacgagatnnnnnnnnatcacgttgtgactggagttcagacgtgtgctcttccgatct

SEQ ID NO: 49 - protein, 413 aa, artificial sequence
  zinc finger targeting B
MDYKDHDGDYKDHDIDYKDDDDKMAPKKKRKVGIHGVPAAMGQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRDKHLNPNEWWKVYPSSVTEFKFLFVSGHFKGNYKAQLTRLNHITNCNGAVLSVEELLIGGEMIKAGTLTLEEVRRKFNNGEINFSGAQGSTLDFRPFQCRICMRNFSRPYTLRLHIRTHTGEKPFACDICGRKFARSANLTRHTKIHTGSQKPFQCRICMRNFSRSDALSTHIRTHTGEKPFACDICGRKFADRSTRTKHTKIHTGEKPFQCRICMRKFADRSTRTKHTKIHLRQKDRSGGGEGRGSLLTCGDVEENPG

SEQ ID NO: 50 - protein, 412 aa, artificial sequence
  zinc finger targeting B
PRTMDYKDHDGDYKDHDIDYKDDDDKMAPKKKRKVGIHGVPAAMAERPFQCRICMQNFSRSDVLSAHIRTHTGEKPFACDICGKKFADRSNRIKHTKIHTGSQKPFQCRICMQNFSDRSHLTRHIRTHTGEKPFACDICGRKFALKQHLTRHTKIHTGEKPFQCRICMQNFSQSGNLARHIRTHTGEKPFACDICGRKFAQSTPRTTHTKIHLRGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINF

SEQ ID NO: 51 - protein, 447 aa, artificial sequence
  zinc finger targeting G
MDYKDHDGDYKDHDIDYKDDDDKMAPKKKRKVGIHGVPAAMGQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRDKHLNPNEWWKVYPSSVTEFKFLFVSGHFKGNYKAQLTRLNHITNCNGAVLSVEELLIGGEMIKAGTLTLEEVRRKFNNGEINFSGTPHEVGVYTLRPFQCRICMRNFSRSDHLSRHIRTHTGEKPFACDICGRKFADSSDRKKHTKIHTGEKPFQCRICMRNFSRSDTLSEHIRTHTGEKPFACDICGRKFAQSGDLTRHTKIHTHPRAPIPKPFQCRICMRNFSQSSDLSRHIRTHTGEKPFACDICGRKFAYKWTLRNHTKIHLRQKDRSGGGEGRGSLLTCGDVEENPG

SEQ ID NO: 52 - protein, 395 aa, artificial sequence
  zinc finger targeting G
PRTMDYKDHDGDYKDHDIDYKDDDDKMAPKKKRKVGIHGVPAAMGQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINFSGTPHEVGVYTLRPFQCRICMRNFSSNQNLTTHIRTHTGEKPFACDICGRKFADRSHLARHTKIHTGEKPFQCRICMQKFAQSGDLTRHTKIHTGEKPFQCRICMQNFSWKHDLTNHIRTHTGEKPFACDICGRKFATSGNLTRHTKIHLRQKD

SEQ ID NO: 53 - protein, 1275 aa, artificial sequence
  ZFN CIITA 87278 DNA
ATGGACTACAAAGACCATGACGGTGATTATAAAGATCATGACATCGATTACAAGGATGACGATGACAAGATGGCCCCCAAGAAGAAGAGGAAGGTCGGCATCCACGGGGTACCCGCCGCTATGGGACAGCTGGTGAAGAGCGAGCTGGAGGAGAAGAAGTCCGAGCTGCGGCACAAGCTGAAGTACGTGCCCCACGAGTACATCGAGCTGATCGAGATCGCCAGGAACAGCACCCAGGACCGCATCCTGGAGATGAAGGTGATGGAGTTCTTCATGAAGGTGTACGGCTACAGGGGAAAGCACCTGGGCGGAAGCAGAAAGCCTGACGGCGCCATCTATACAGTGGGCAGCCCCATCGATTACGGCGTGATCGTGGACACAAAGGCCTACAGCGGCGGCTACAATCTGCCTATCGGCCAGGCCGACGAGATGGAGAGATACGTGGAGGAGAACCAGACCCGGGATAAGCACCTCAACCCCAACGAGTGGTGGAAGGTGTACCCTAGCAGCGTGACCGAGTTCAAGTTCCTGTTCGTGAGCGGCCACTTCAGCGGCAACTACAAGGCCCAGCTGACCAGGCTGAACCACATCACCAACTGCAATGGCGCCGTGCTGAGCGTGGAGGAGCTGCTGATCGGCGGCGAGATGATCAAAGCCGGCACCCTGACACTGGAGGAGGTGCGGCGCAAGTTCAACAACGGCGAGATCAACTTCAGCGGCACTCCACACGAAGTGGGAGTGTACACACTTAGGCCCTTCCAGTGTCGAATCTGCATGCGTAACTTCAGTCGTAGTGACCACCTGAGCCGGCACATCCGCACCCACACAGGCGAGAAGCCTTTTGCCTGTGACATTTGTGGGAGGAAATTTGCCGACAGCAGCGACCGCAAAAAGCATACCAAGATACACACGGGCGAGAAGCCCTTCCAGTGTCGAATCTGCATGCGTAACTTCAGTCGCTCCGACACCCTGTCCGAGCACATCCGCACCCACACCGGCGAGAAGCCTTTTGCCTGTGACATTTGTGGGAGGAAATTTGCCCAGTCCGGCGACCTGACCCGCCATACCAAGATACACACGCACCCGCGCGCCCCGATCCCGAAGCCCTTCCAGTGTCGAATCTGCATGCGTAACTTCAGTCAGTCCTCCGACCTGTCCCGCCACATCCGCACCCACACCGGCGAGAAGCCTTTTGCCTGTGACATTTGTGGGAGGAAATTTGCCTACAAGTGGACCCTGCGCAACCATACCAAGATACACCTGCGGCAGAAGGAC

SEQ ID NO: 54 - protein, 425 aa, artificial sequence
  ZFN CIITA 87278 protein
MDYKDHDGDYKDHDIDYKDDDDKMAPKKKRKVGIHGVPAAMGQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRDKHLNPNEWWKVYPSSVTEFKFLFVSGHFSGNYKAQLTRLNHITNCNGAVLSVEELLIGGEMIKAGTLTLEEVRRKFNNGEINFSGTPHEVGVYTLRPFQCRICMRNFSRSDHLSRHIRTHTGEKPFACDICGRKFADSSDRKKHTKIHTGEKPFQCRICMRNFSRSDTLSEHIRTHTGEKPFACDICGRKFAQSGDLTRHTKIHTHPRAPIPKPFQCRICMRNFSQSSDLSRHIRTHTGEKPFACDICGRKFAYKWTLRNHTKIHLRQKD

SEQ ID NO: 55 - DNA, 1053 bp, artificial sequence
  ZFN CIITA 87232 DNA
cagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctaccggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttcagcggcactccacacgaagtgggagtgtacacacttaggcccttccagtgtcgaatctgcatgcgtaacttcagttccaaccagaacctgaccacccacatccgcacccacaccggcgagaagccttttgcctgtgacatttgtgggaggaaatttgccgaccgctcccacctggcccgccataccaagatacacacgggcgagaagcccttccagtgtcgaatctgcatgcagaagtttgcccagtccggcgacctgacccgccataccaagatacacacgggcgagaagcccttccagtgtcgaatctgcatgcagaacttcagttggaagcacgacctgaccaaccacatccgcacccacaccggcgagaagccttttgcctgtgacatttgtgggaggaaatttgccacctccggcaacctgacccgccataccaagatacacctgcggcagaaggactga

SEQ ID NO: 56 - protein, 350 aa, artificial sequence
  ZFN CIITA 87232 protein
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPTGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINFSGTPHEVGVYTLRPFQCRICMRNFSSNQNLTTHIRTHTGEKPFACDICGRKFADRSHLARHTKIHTGEKPFQCRICMQKFAQSGDLTRHTKIHTGEKPFQCRICMQNFSWKHDLTNHIRTHTGEKPFACDICGRKFATSGNLTRHTKIHLRQKD

SEQ ID NO: 57 - DNA, 5670 bp, Artificial Sequence
  Plasmid
gctgcttcgcgatgtacgggccagatatacgcgcatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctaatacgactcactatagggagacaagcttgaatacaagcttgcttgttctttttgcagaagctcagaataaacgctcaactttggcagatcgaattcgccatggactacaaagaccatgacggtgattataaagatcatgacatcgattacaaggatgacgatgacaagatggcccccaagaagaagaggaaggtcggcatccacggggtacccgccgctatgggacagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcagcggcaactacaaggcccagctgaccaggctgaaccacatcaccaactgcaatggcgccgtgctgagcgtggaggagctgctgatcggcggcgagatgatcaaagccggcaccctgacactggaggaggtgcggcgcaagttcaacaacggcgagatcaacttcagcggcactccacacgaagtgggagtgtacacacttaggcccttccagtgtcgaatctgcatgcgtaacttcagtcgtagtgaccacctgagccggcacatccgcacccacacaggcgagaagccttttgcctgtgacatttgtgggaggaaatttgccgacagcagcgaccgcaaaaagcataccaagatacacacgggcgagaagcccttccagtgtcgaatctgcatgcgtaacttcagtcgctccgacaccctgtccgagcacatccgcacccacaccggcgagaagccttttgcctgtgacatttgtgggaggaaatttgcccagtccggcgacctgacccgccataccaagatacacacgcacccgcgcgccccgatcccgaagcccttccagtgtcgaatctgcatgcgtaacttcagtcagtcctccgacctgtcccgccacatccgcacccacaccggcgagaagccttttgcctgtgacatttgtgggaggaaatttgcctacaagtggaccctgcgcaaccataccaagatacacctgcggcagaaggacagatctggcggcggagagggcagaggaagtcttctaacctgcggtgacgtggaggagaatcccggccctaggaccatggactacaaagaccatgacggtgattataaagatcatgacatcgattacaaggatgacgatgacaagatggcccccaagaagaagaggaaggtcggcattcatggggtacccgccgctatgggacagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctaccggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttcagcggcactccacacgaagtgggagtgtacacacttaggcccttccagtgtcgaatctgcatgcgtaacttcagttccaaccagaacctgaccacccacatccgcacccacaccggcgagaagccttttgcctgtgacatttgtgggaggaaatttgccgaccgctcccacctggcccgccataccaagatacacacgggcgagaagcccttccagtgtcgaatctgcatgcagaagtttgcccagtccggcgacctgacccgccataccaagatacacacgggcgagaagcccttccagtgtcgaatctgcatgcagaacttcagttggaagcacgacctgaccaaccacatccgcacccacaccggcgagaagccttttgcctgtgacatttgtgggaggaaatttgccacctccggcaacctgacccgccataccaagatacacctgcggcagaaggactgataactcgagtctagaagctcgctttcttgctgtccaatttctattaaaggttcctttgttccctaagtccaactactaaactgggggatattatgaagggccttgagcatctggattctgcctaataaaaaacatttattttcattgctgcgctagaagctcgctttcttgctgtccaatttctattaaaggttcctttgttccctaagtccaactactaaactgggggatattatgaagggccttgagcatctggattctgcctaataaaaaacatttattttcattgctgcgggacattcttaattaaaaaaaaaaaaaaaaaaaaaaaaaaaaaaaaaaaaaaaaaaaaaaaaaaaaaaaaaaaactagtggcgcctgatgcggtattttctccttacgcatctgtgcggtatttcacaccgcataatccagcacagtggcggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt